(19) United States
(12) Patent Application Publication
(10) Pub. No.: US 2025/0382625 A1
(43) Pub. Date: Dec. 18, 2025

(54) SYNTHETIC PRE-PROTEIN SIGNAL PEPTIDES FOR DIRECTING SECRETION OF HETEROLOGOUS PROTEINS IN ESCHERICHIA BACTERIA

(71) Applicant: TENZA, INC., Boston, MA (US)

(72) Inventor: Anik Debnath, Somerville, MA (US)

(21) Appl. No.: 18/864,992

(22) PCT Filed: May 12, 2023
(86) PCT No.: PCT/US2023/066925

§ 371 Date: Nov. 12, 2024

Related U.S. Application Data
(60) Provisional application No. 63/340,963, filed on May 12, 2022.

(51) Int. Cl. C12N 15/62; C12N 1/20; C12R 1/185

(52) U.S. Cl. CPC C12N 15/625; C12N 1/20; C12R 2001/185

(57) ABSTRACT

Provided herein are pre-protein signal peptides that direct secretion of expressed payload proteins to the periplasm in Escherichia bacteria and methods of their use in production of therapeutic and industrial commodity proteins. The disclosed pre-protein signal peptides may be used with any pay load protein to increase secretion thereof and therefore increase yield of the payload protein.

### CROSS REFERENCE TO RELATED APPLICATIONS

This application claims the benefit of U.S. Provisional Application Ser. No. 63/340,963 filed May 12, 2022, which is hereby incorporated by reference in its entirety.

### REFERENCE TO SEQUENCE LISTING SUBMITTED ELECTRONICALLY

The instant application contains a Sequence Listing which has been submitted electronically in XML format and is hereby incorporated by reference in its entirety. Said XML copy, created on May 10, 2023, is named TNZ-006WO_SL.XML and is 200,059 bytes in size.

### FIELD

The present disclosure relates generally to signal peptides and more particularly to synthetic pre-protein signal peptides that increase secretion of a recombinant protein in Escherichia.

### BACKGROUND

Bacteria are routinely used as hosts to produce proteins for research, therapeutic and industrial purposes. The first step during the secretion of a desired target protein into the growth medium is its transport across the cytoplasmic membrane. In bacteria, two major export pathways, the general secretion or Sec pathway and the twin-arginine translocation or Tat pathway, exist for the transport of proteins across the plasma membrane. The routing into one of these alternative protein export systems requires the fusion of a Sec- or Tat-specific signal peptide to the amino-terminal end of the desired target protein. Since signal peptides, besides being required for the targeting to and membrane translocation by the respective protein translocases, also have additional influences on the biosynthesis, the folding kinetics, and the stability of the respective payload proteins, it is not possible so far to predict in advance which signal peptide will perform best in the context of a given target protein and a given bacterial expression host.

The secretion of recombinant proteins into the growth medium of the respective bacterial host organisms possesses several important benefits compared to intracellular expression strategies. First, secretion of aggregation-prone proteins can prevent their accumulation as insoluble inclusion bodies in the cytosol. Second, the toxic effect exerted by some proteins on the production host upon their intracellular expression can be reduced or even be alleviated when the respective protein is secreted out of the cell into the surrounding culture medium. Third, since many interesting payload proteins (e.g. therapeutic antibodies) require the correct formation of disulfide bonds for their final conformations and biological activities, the secretion of the respective proteins into an extra cytoplasmic compartment is an essential step for their production since disulfide bond formation is effectively prevented in the reducing environment of the cytosol. Finally, and most importantly, the secretion of a desired payload protein into the growth medium greatly simplifies product recovery, since no cell disruption is required and the subsequent purification and downstream processing steps can be significantly reduced.

Although driving secretion of recombinant proteins into the growth medium is beneficial at least for the reasons recited above, driving secretion of recombinant proteins from the cytoplasm to an extra cytoplasmic compartment (i.e., periplasm) can also be beneficial. As highlighted above, disulfide containing proteins may not fold correctly in the cytosol of the host organism due to the reducing environment of the cytosol. In such instances, driving protein expression to the periplasm can promote proper folding of the recombinant protein. Although the protein is not excreted to the growth medium, isolation of a recombinant protein from the periplasm can help simplify the purification process as the protein is isolated from a mixture that is less complex (i.e., periplasmic fraction rather than whole cell). Techniques for isolation of a recombinant protein from the periplasm are well known and can also be performed at industrial scale. Due to this, the secretory production of a given payload protein, either to the growth medium or an extra cytoplasmic compartment such as the periplasm, can drastically decrease the overall production costs

Escherichia bacteria, especially E. coli, are extensively used in industry for the production of a variety of technical enzymes such as lipases, amylases, and proteases, resulting in high production yields. However, these exceptional high product yields are obtained predominantly only for naturally secreted enzymes that originate either directly from the production host itself or from one of its close relatives. In contrast, the yields obtained for heterologous proteins are often comparably very low or the desired target proteins were not secreted at all. Secretion, particularly for disulfide laden proteins, has been a long-standing bottleneck against increased yields. This is due to the lack of predictability of what signal peptides function best for any given product, and due to the perceived saturation limit of protein secretion machinery past which loss of bacteria strain fitness reduces overall biomass. A need therefore exists for engineering a system that not only increases the secretion of a non-native recombinant protein in Escherichia bacteria, but has application across numerous bacteria species. The embodiments of the present disclosure address these needs and others. In particular, the signal peptides of the present disclosure are optimized to function as universal signal peptides and are designed in a manner that accounts for bacteria strain fitness for the purposes of maximizing not just yield per cell, but also yield per batch.

### SUMMARY

In some embodiments, a pre-protein signal peptide is provided. In some embodiments, the pre-protein signal peptide comprises an amino acid sequence of Formula I, wherein Formula I is represented as:

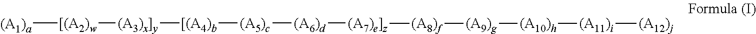

wherein:
   each w is, independently, 1 or 2;
   each x is, independently, 0 or 1;
   y is 1, 2, or 3;
   z is 2, 3, 4, 5, 6, 7, 8, 9, 10, 11, or 12;
   each b, c, d, and e are each, independently, 0 or 1; and
   a, f, g, h, i, and j are each, independently, 0 or 1,
wherein:
   A₁ is methionine
   each A₂ is, independently, an amino acid selected from the group consisting of K, R, N, A, P, S, T, I, and F;
   each A₃ is, independently, an amino acid selected from the group consisting of I, L, F, V, M, Y, and H;
   each A₄ is, independently, an amino acid selected from the group consisting of L, V, C, A, F, I, T, M, P, S, G, W, Y, Q, N, R, and H;
   each A₅ is, independently, an amino acid selected from the group consisting of A, G, S, T, P, M, C, V, W, I, L, F, Y, Q, N, R, E, K, D, and H;
   each A₆ is, independently, an amino acid selected from the group consisting of G, S, N, and Q;
   each A₇ is, independently, an amino acid selected from the group consisting of S, N, T, G, V, I, Q, A, C, P, Y, M, F, and L;
   A₈ is an amino acid selected from the group consisting of A, T, G, S, P, V, I, L, Q, R, M, W, F, C, N, K, E, D, and H;
   A₉ is an amino acid selected from the group consisting of Q, F, N, S, E, T, D, R, H, K, G, A, P, Y, M, V, W, I, and L;
   A₁₀ is an amino acid selected from the group consisting of A, T, G, S, P, V, I, L, Q, R, M, W, F, C, N, K, E, D, and H;
   A₁₁ is an amino acid selected from the group consisting of A, T, G, S, P, V, I, L, Q, R, M, W, F, C, N, K, E, D, and H; and
   A₁₂ is an amino acid selected from the group consisting of D, E, Q, N, S, H, T, R, K, G, A, C, Y, P, M, V, W, I, and L.

In some embodiments, a pre-protein signal peptide is provided. In some embodiments, the pre-protein signal peptide comprises an amino acid sequence of Formula I, wherein Formula I is represented as:

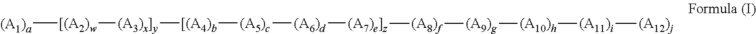

wherein:
   each w is, independently, 1 or 2;
   each x is, independently, 0 or 1;
   y is 1, 2, or 3;
   z is 2, 3, 4, 5, 6, 7, 8, 9, 10, 11, or 12;
   each b, c, d, and e are each, independently, 0 or 1; and
   a, f, g, h, i, and j are each, independently, 0 or 1,
wherein:
   A₁ is methionine
   each A₂ is, independently, an amino acid selected from the group consisting of K, R, and N;
   each A₃ is, independently, isoleucine (I);
   each A₄ is, independently, an amino acid selected from the group consisting of L, V, C, and A;
   each A₅ is, independently, an amino acid selected from the group consisting of A, G, and S;
   each A₆ is, independently, an amino acid selected from the group consisting of G, S, N, and Q;
   each A₇ is, independently, an amino acid selected from the group consisting of S, N, T, G, V, and I;
   A₈ is an amino acid selected from the group consisting of A, T, G, and S;
   A₉ is an amino acid selected from the group consisting of Q and F;
   A₁₀ is an amino acid selected from the group consisting of A, T, G, and S;
   A₁₁ is an amino acid selected from the group consisting of A, T, G, and S; and
   A₁₂ is an amino acid selected from the group consisting of D, E, Q, N, S, H, T, R, K, G, A, C, Y, P, M, V, W, I, and L.

In some embodiments, the pre-protein signal peptide comprises an amino acid sequence having at least 90%, 91%, 92%, 93%, 94%, 95%, 96%, 97%, 98%, or 99% identity to an amino acid sequence of SEQ ID NO: 1, 2, 3, or 4.

In some embodiments, the pre-protein signal peptide increases the secretion of a payload protein as compared to native signal peptides.

In some embodiments, a polypeptide is provided. In some embodiments, the polypeptide comprises a formula of X₁—Z₁, wherein X₁ is a pre-protein signal peptide, and Z₁ is a payload protein. In some embodiments, X₁ comprises an amino acid sequence of Formula I as provided for herein. In some embodiments, X₁ comprises an amino acid sequence having at least 90%, 91%, 92%, 93%, 94%, 95%, 96%, 97%, 98%, or 99% identity to an amino acid sequence of SEQ ID NO: 1, 2, 3, or 4. In some embodiments, the pre-protein signal peptide of X₁ increases the secretion of a payload protein as compared to native signal peptides. In some embodiments, Z₁ is as provided for herein. In some embodiments, Z₁ comprises an amino acid sequence having at least 90%, 91%, 92%, 93%, 94%, 95%, 96%, 97%, 98%, or 99% identity to an amino acid sequence of SEQ ID NO: 28, 29, 30, 31, 32, 33, 34, 35, 36, 37, 38, 39, 40, 41, 42, 43, 44, or 72.

In some embodiments, a bacterium is provided. In some embodiments, the bacterium comprises a heterologous nucleic acid molecule encoding a polypeptide having a formula of X₁—Z₁, wherein X₁ is a pre-protein signal peptide, and Z₁ is a payload protein.

In some embodiments, a method for producing a payload protein is provided. In some embodiments, the method comprises transfecting a bacterium with a nucleic acid molecule encoding for a recombinant polypeptide as provided for herein to produce a bacterium comprising the nucleic acid molecule, culturing the bacteria comprising the nucleic acid molecule under conditions sufficient to grow the bacteria, and inducing secretion of the payload protein by the bacteria.

In some embodiments, a method for producing an industrial commodity protein is provided. In some embodiments, the method comprises transfecting a bacterium with a nucleic acid molecule encoding for a recombinant polypeptide comprising a formula of X₁—Z₁, wherein X₁ is a pre-protein signal peptide and Z₁ is a payload protein comprising an industrial commodity protein, thereby producing a bacterium comprising the nucleic acid molecule, culturing the bacteria comprising the nucleic acid molecule under conditions sufficient to grow the bacteria, and inducing secretion of the payload protein by the bacteria.

In some embodiments, a method for treating a disease or a condition in a subject in need thereof is provided. In some embodiments, the method comprises administering to the subject a therapeutically effective amount of a bacteria as provided for herein.

### BRIEF DESCRIPTION OF THE FIGURES

FIG. 1 illustrates the results of a luciferase secretion assay comparing the synthetic signal peptides provided herein to known signal peptides.

### DETAILED DESCRIPTION

The present disclosure presents a solution to the aforementioned challenges by providing new, synthetic signal peptides that direct secretion of expressed proteins or peptides in Escherichia bacteria. The disclosed signal peptides overcome performance variability challenges posed by previously characterized and native signal peptides and may be used to generate and facilitate secretion of any protein or peptide from bacteria.

The disclosed synthetic pre-protein signal peptides increase secretion of any recombinant protein in Escherichia bacteria. The use of synthetic pre-protein signal peptide may further improve secretion of a payload protein, for example, through facilitating translocation across the cytoplasmic membrane. Advantageously, the signal peptides disclosed herein have been generated and optimized to promote secretion of any payload protein from Escherichia bacteria. Use of the disclosed synthetic pre-protein signal peptides may be used to achieve increased secretion of any desired payload to any bacteria-compatible environment, such as in therapeutics, agriculture, or food products.

Before the present compositions and methods are described, it is to be understood that the scope of the invention is not limited to the particular processes, compositions, or methodologies described herein, as these may vary. It is also to be understood that the terminology used in the description is for the purpose of describing the particular versions or embodiments only, and is not intended to limit the scope of the present invention. Unless defined otherwise, all technical and scientific terms used herein have the same meanings as commonly understood by one of ordinary skill in the art. Although any methods and materials similar or equivalent to those described herein can be used in the practice or testing of embodiments of the methods and systems disclosed herein, the preferred methods, devices, and materials are now described.

### Definitions

The following explanations of terms and methods are provided to better describe the present disclosure and to guide those of ordinary skill in the art in the practice of the present disclosure.

As used herein, “comprising” means “including” and the singular forms “a” or “an” or “the” include plural references unless the context clearly dictates otherwise. For example, reference to “comprising a therapeutic agent” includes one or a plurality of such therapeutic agents. The term “or” refers to a single element of stated alternative elements, unless the context clearly indicates otherwise. For example, the phrase “A or B” refers to A alone or B alone. The phrase “A, B, or a combination thereof” refers to A alone, B alone, or a combination of A and B. Similarly. “one or more of A and B” refers to A, B, or a combination of both A and B. The phrase “A and B” refers to a combination of A and B. Furthermore, the various elements, features and steps discussed herein, as well as other known equivalents for each such element, feature or step, can be mixed and matched by one of ordinary skill in this art to perform methods in accordance with principles described herein. Among the various elements, features, and steps some will be specifically included and others specifically excluded in particular examples.

Unless otherwise defined, all technical and scientific terms used herein have the same meaning as commonly understood to one of ordinary skill in the art. Although methods and materials similar or equivalent to those described herein can be used in the practice or testing of the present disclosure, suitable methods and materials are described below. The materials, methods, and examples are illustrative only and not intended to be limiting. All references cited herein are incorporated by reference in their entirety.

In some examples, the numbers expressing quantities of ingredients, properties such as molecular weight, reaction conditions, and so forth, used to describe and claim certain embodiments are to be understood as being modified in some instances by the term “about” or “approximately.” For example, “about” or “approximately” can indicate +/−5% variation of the value it describes. Accordingly, in some embodiments, the numerical parameters set forth herein are approximations that can vary depending upon the desired properties for a particular embodiment. Notwithstanding that the numerical ranges and parameters setting forth the broad scope of some examples are approximations, the numerical values set forth in the specific examples are reported as precisely as practicable. The recitation of ranges of values herein is merely intended to serve as a shorthand method of referring individually to each separate value falling within the range.

To facilitate review of the various embodiments of this disclosure, the following explanations of specific terms are provided:

As used herein, “bacteria” (plural) and “bacterium” (singular) refer to a unicellular prokaryotic microorganism. Bacteria cells are generally surrounded by two protective coatings: an outer cell wall and an inner cell membrane. Bacteria may be classified according to the Gram stain, which identifies bacteria by the composition of their cell walls. Gram-positive bacteria do not have an outer membrane whereas Gram-negative bacteria do not. Bacteria generally reproduce by binary fission, where a parent cell makes a copy of its DNA and grows larger by doubling its cellular content. The cell then splits apart, pushing the extra cellular content out, creating two daughter cells. Some bacteria utilize other processes, such as budding. In some embodiments, the bacteria are wild-type natural isolates of bacteria. In some embodiments, the bacteria are laboratory strains of bacteria that have undergone domestication processes of mutagenesis and selection. As used herein, “bacteria” refers to any wild type or laboratory strain of bacteria known.

As used herein, “Escherichia bacteria” refer to a genus of rod-shaped, gram-positive aerobic or anaerobic bacteria that are widely found in soil and water. Examples of Escherichia bacteria include, but are not limited to E. albertii, E. coli, E. fergusonii, E. hermannii, E. senegalensis, E. marmotae, E. ruysiae, and E. vulneris. In some embodiments, the Escherichia bacteria are wild-type natural isolates of Escherichia. In some embodiments, the Escherichia bacteria are laboratory strains of Escherichia that have undergone domestication processes of mutagenesis and selection, for example, but not limited to, MG1655, NEB Turbo, DH10B, NEB Stable, DH5α, Mach1, BW25113, DB3.1, OmniMAX2, XL1-Blue, NEB dam⁻/dcm⁻, ET12567, EC100D, BW25141, BW2474, BW29655, Marionette-Clo, Marionette-Pro, Marionette-Wild, BL21 (DE3). Rosetta™ (DE3)pLysS, BLIM, BioDesignER (RE1000), Nissle 1917, DH1, JM109, BLR(DE3), BLR(DE3) pRIL, DP10, RU1012, JTK165JJ, BW27783, DGF-298, K-12 strain 58, K-12 strain 679, K12-strain WG1, K-12 derivative strains 5K, 58, 58-161, AN284, AB311, AG1, C600, DP50, EMG2, EPI100-T1R, H1443, HB101, Hfr3000, Hfr 3000X74, JM109, TG1, TOP10, W1485, W208, W3110, W945, WA704, WG1, JC9387, JM83, JM101, KP7600, LE392, M15, MB408, Novablue, P678, PA 309, REG-12, S17-1, SCS-110, SM10, STBL2, STBL3, TB1, SURE, XL10-Glod, XLOLR, T10, and YN2980, and non-K12 strains B, B-3, B/R, BL23, C, C41, C43, FDA strain Seattle 1946, K5808, Nissle 1917, Rosetta, REG-811, W, and 25922. As used herein, “Escherichia bacteria” refers to any wild type or laboratory strain of Escherichia bacteria known. Further, in referring to any specific Escherichia species, the recitation of the species also includes any wild type or laboratory strain of the Escherichia species know. Thus, for example, when referring to E. coli it is to be understood that “E. coli” encompasses wild type E. coli as well as laboratory strains of E. coli, such as, but not limited to, MG1655, NEB Turbo, DH10B, NEB Stable, DH5α, Mach1, BW25113, DB3.1, OmniMAX2, XL1-Blue, NEB dam⁻/dcm⁻, ET12567, EC100D, BW25141, BW2474, BW29655, Marionette-Clo, Marionette-Pro, Marionette-Wild, BL21 (DE3), Rosetta™ (DE3) pLysS, BLIM, BioDesignER (RE1000), Nissle 1917, DH1, JM109, BLR(DE3), BLR(DE3) pRIL, DP10, RU1012, JTK165JJ, BW27783, DGF-298, K-12 strain 58, K-12 strain 679, K12-strain WG1, K-12 derivative strains 5K, 58, 58-161, AN284, AB311, AG1, C600, DP50, EMG2, EPI100-T1R, H1443, HB101, Hfr3000, Hfr 3000 X74, JM109, TG1, TOP10, W1485, W208, W3110, W945, WA704, WG1, JC9387, JM83, JM101, KP7600, LE392, M15, MB408, Novablue, P678, PA 309, REG-12, S17-1, SCS-110, SM10, STBL2, STBL3, TB1, SURE, XL10-Glod, XLOLR, T10, and YN2980, and non-K12 strains B, B-3, B/R, BL23, C, C41, C43, FDA strain Seattle 1946, K5808, Nissle 1917, Rosetta, REG-811, W, and 25922.

As used herein, “genetically modified” or any grammatical variation thereof, refers to a practice of introducing a nucleic acid into a bacterial cell that encodes to promote the expression of a recombinant protein therein. A nucleic acid may be DNA, mRNA, tRNA, or rRNA. A nucleic acid is composed of nucleotide monomers, each triplet of monomers (a codon) encoding for either a triplet of RNA nucleotide monomers (if the nucleic acid is DNA) or an amino acid (if the nucleic acid is RNA). DNA also comprises one or more promoter regions, which indicate where transcription of the DNA should start. mRNA also comprises a ribosome binding site, which indicates where translation of the mRNA should start as well as one or more stop codons, which indicates where mRNA translation should end.

In any embodiment or aspect disclosed herein, a nucleic acid encoding for a recombinant fusion protein, as disclosed herein, may be introduced into a bacterial cell using any method known to those skilled in the art for such introduction. Such methods include transfection, transformation, transduction, infection (e.g., viral transduction), injection, microinjection, gene gun, nucleofection, nanoparticle bombardment, transformation, conjugation, by application of the nucleic acid in a gel, oil, or cream, by electroporation, using lipid-based transfection reagents, or by any other suitable transfection method. One of skill in the art will readily understand and adapt such methods using readily identifiable literature sources.

As used herein, the terms “transformation” and “transfection” are intended to refer to a variety of art-recognized techniques for introducing foreign nucleic acid into a host cell, including calcium phosphate or calcium chloride co-precipitation, DEAE-dextran-mediated transfection, lipofection (e.g., using commercially available reagents such as, for example, LIPOFECTIN® (Invitrogen Corp., San Diego, CA), LIPOFECTAMINE® (Invitrogen), FUGENE® (Roche Applied Science, Basel, Switzerland), JETPEI™ (Polyplus-transfection Inc., New York, NY), EFFECTENE® (Qiagen, Valencia, CA), DREAMFECT™ (OZ Biosciences, France) and the like), or electroporation (e.g., in vivo electroporation). Suitable methods for transforming or transfecting host cells can be found in Sambrook, et al. (Molecular Cloning: A Laboratory Manual. 2^{nd}, ed., Cold Spring Harbor Laboratory, Cold Spring Harbor Laboratory Press, Cold Spring Harbor, N.Y., 1989), and other laboratory manuals.

Methods and materials of non-viral delivery of nucleic acids to cells further include biolistics, virosomes, liposomes, immunoliposomes, polycation or lipid-nucleic acid conjugates, naked DNA, artificial virions, and agent-enhanced uptake of DNA. Lipofection is described in U.S. Pat. Nos. 5,049,386, 4,946,787; and 4,897,355 and lipofection reagents are sold commercially (e.g., TRANSFECTAM™ and LIPOFECTIN™). Cationic and neutral lipids that are suitable for efficient receptor-recognition lipofection of polynucleotides include those disclosed in WO 91/17424 and WO 91/16024.

The methods described herein comprise generating a recombinant fusion protein within a bacterial host. As used herein, heterologous or recombinant describes a protein or nucleic acid that is not naturally found in or produced by the host bacteria. As used herein, a “recombinant fusion protein” comprises a payload protein and a synthetic signal peptide fused directly or indirectly thereto. As used herein, a signal peptide is any protein or peptide fused directly or indirectly to the N-terminus of a payload protein that facilitates the extracellular secretion of the payload protein after it is generated.

The chemical makeup of a peptide will be described herein by a series of amino acid single letter abbreviations or an “amino acid sequence/s” or “sequence/s,” which are conventional and known to those in the art. While reference sequences will be explicitly disclosed, in any aspect and embodiment, a reference sequence may be modified to include conservative amino acid substitutions, as well as variants and fragments, while maintaining the characteristics and functionality of the reference sequence.

As used herein, the terms “secretion”, “secreted”, or any other form thereof refers to any peptide or protein that is exported based on the presence of a secretory signal peptide, artificial or otherwise. It is to be understood that “secretion”, “secreted”, etc., does not refer to a specific peptide or protein destination. Therefore, in some embodiments, a “secreted” peptide or protein may be exported to the culture media. In some embodiments, a “secreted” peptide or protein may be exported to the periplasm. In the context of the present disclosure, unless explicitly denoted otherwise, the terms “secretion”, “secreted”, or any other form thereof encompasses all peptide or protein destinations as a result of the presence of a secretory signal peptide. Thus, for clarity, when considering the phrase “wherein the presence of the pre-protein signal peptide induces secretion of the payload protein”, such a phrase encompasses i) export of the payload protein to the culture media, and ii) export of the protein to the periplasm.

The methods disclosed herein utilize a synthetic signal peptide to increase extracellular secretion of a payload protein by a bacterium. As used herein, a “synthetic signal peptide” refers to a signal peptide whose sequence is generated as provided for herein and is made recombinantly. The recombinantly produced signal peptide can be referred to as a “synthetic signal peptide” or simply as a “signal peptide”. In some embodiments, the signal peptide may comprise a synthetic pre-protein signal peptide. As highlighted previously, the term synthetic in this context refers to a recombinantly produced pre-protein signal peptide whose sequence is generated as provided for herein. Hereafter, the pre-signal peptide may be referred to as a “synthetic” pre signal peptide, or simply as a pre-protein signal peptide. In embodiments where a native pre-protein signal peptide is utilized or referred to, the peptide will be denoted as such. In the context of this application, the term “native” refers to a pre-protein signal peptide the sequence of which is adopted, in whole or in part, from a known pre-protein signal peptide sequence at the time of this application. In other words, the “native” signal peptides are not generated using the formulas or methods as provided for herein.

A pre-protein signal peptide (synthetic or native) comprises 10 to 50 amino acids, which are appended either directly to the N-terminus of a payload protein or indirectly (e.g., using one or more spacers) to the N-terminus of a payload protein.

A synthetic pre-protein signal peptide may be appended to an adjacent amino acid via a bond to the N-terminal amino acid of the adjacent amino acid, for example, by a peptide bond, a peptide spacer (e.g., LEISSTCDA, represented by SEQ ID NO: 9, or a membrane-associating/lipidophilic alpha-helical peptide signal peptide (e.g., MISTIC, represented by SEQ ID NO: 11).

As used herein, “payload protein” or “protein of interest” refers to the protein that will be generated by the host and chaperoned through the secretory pathway into the extracellular space or to the bacterial periplasm, facilitated by the presence of a synthetic signal peptide. Upon secretion into the extracellular space or to the periplasm, all, some, or none of the synthetic signal peptide may be fused to the payload protein. Optionally, a payload protein still being attached partially or fully to the synthetic signal peptide may be further processed, for example, to remove the remaining signal peptide. A payload protein may be any protein known or yet to be known, for example, an enzyme, enzyme inhibitor, growth factor, hormone, antibody, antigen, vaccine, a therapeutic agent, or any combination thereof. More specific examples follow herein below.

As used herein, “substantially identical” or “substantially similar” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

Sequence identity can be measured/determined using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e3 and e100 indicating a closely related sequence. In some embodiments, sequence identity is determined by using BLAST with the default settings.

To the extent embodiments provided for herein, includes composition comprising various proteins, these proteins may, in some instances, comprise amino acid sequences that have sequence identity to the amino acid sequences disclosed herein. Therefore, in certain embodiments, depending on the particular sequence, the degree of sequence identity is preferably greater than 50% (e.g. 60%, 70%, 75%, 80%, 85%, 90%, 91%, 92%, 93%, 94%, 95%, 96%, 97%, 98%, 99% or more) to the SEQ ID NOs disclosed herein. These proteins may include homologs, orthologues, allelic variants and functional mutants. Typically, 50% identity or more between two polypeptide sequences is considered to be an indication of functional equivalence. Identity between polypeptides is preferably determined by the Smith-Waterman homology search algorithm as implemented in the MPSRCH program (Oxford Molecular), using an affine gap search with parameters gap open penalty−12 and gap extension penalty=1.

These proteins may, compared to the disclosed proteins, include one or more (e.g. 1, 2, 3, 4, 5, 6, 7, 8, 9, 10, etc.) conservative amino acid replacements i.e. replacements of one amino acid with another which has a related side chain. Genetically-encoded amino acids are generally divided into four families: (1) acidic i.e. aspartate, glutamate; (2) basic i.e. lysine, arginine, histidine; (3) non polar i.e. alanine, valine, leucine, isoleucine, proline, phenylalanine, methionine, tryptophan; and (4) uncharged polar i.e. glycine, asparagine, glutamine, cysteine, serine, threonine, tyrosine. Phenylalanine, tryptophan, and tyrosine are sometimes classified jointly as aromatic amino acids. In general, Substitution of single amino acids within these families does not have a major effect on the biological activity. The proteins may have one or more (e.g. 1, 2, 3, 4, 5, 6, 7, 8, 9, 10, etc.) single amino acid deletions relative to the disclosed protein sequences. The proteins may also include one or more (e.g. 1, 2, 3, 4, 5, 6, 7, 8, 9, 10, etc.) insertions (e.g. each of 1, 2, 3, 4 or 5 amino acids) relative to the disclosed protein sequences.

The compositions disclosed herein may be provided to a subject in a variety of ways through administration of the composition to the subject. As used herein, administer or administration means to provide or the providing of a composition to a subject. Oral administration, as used herein, refers to delivery of an active agent through the mouth. Topical administration, as used herein, refers to the delivery of an active agent to a body surface, such as the skin, a mucosal membrane (e.g., nasal membrane, vaginal membrane, buccal membrane, or the like).

As used herein, “hydropathy index” or “HP index” refers to the “intrinsic” hydrophobicity/hydrophilicity of amino acid side chains in peptides/proteins as defined in Kovacs J M, Mant C T, Hodges R S. Determination of intrinsic hydrophilicity/hydrophobicity of amino acid side chains in peptides in the absence of nearest-neighbor or conformational effects. Biopolymers 2006; 84(3):283-97. Doi: 10.1002/bip.20417. PMID: 16315143; PMCID: PMC2744689, which is hereby incorporated by reference in its entirety. Hydrophobicity/hydrophilicity values were determined via a synthetic peptide wherein the HP index value is calculated as the difference in RP-HPLC retention time between amino acid X at the i position and amino acid Gly at the i+1 position. Thus, amino acids that are more hydrophobic than glycine have a positive HP index value and amino acids that are more hydrophilic than glycine have a negative HP index value, wherein glycine would have a 0 value. See Table 1 below, values which correspond to the values utilized for the present application:

TABLE 1

| | pH 5, 10 mM PO₄ Buffer |
|---|---|
| Amino Acid Substitution | Δt_{R(Gly)} |
| Trp (W) | 33.2 |
| Phe (F) | 30.1 |
| Leu (L) | 24.1 |
| Ile (I) | 22.2 |
| Met (M) | 16.4 |
| Tyr (Y) | 15.2 |
| Val (V) | 14.0 |
| Pro (P) | 9.4 |
| Cys (C) | 7.9 |
| Ala (A) | 3.3 |
| Glu (E) | −0.5 |
| Thr (T) | 2.8 |
| Asp (D) | −1.0 |
| Gln (Q) | 0.6 |
| Ser (S) | 0.0 |
| Asn (N) | 0.0 |
| Gly (G) | 0.0 |
| Arg (R) | −3.7 |
| His (H) | −5.1 |
| Lys (K) | −3.7 |

As used herein “helicity” refers to the nonpolar phase helical propensity of each guest “X” residue in an experimental KKAAAXAAAAAXAAWAAXAAAKKKK (SEQ ID NO: 16)-amide peptide, as outlined in Deber C M. Wang C, Liu L P, Prior A S, Agrawal S, Muskat B L, Cuticchia A J. TM Finder: a prediction program for transmembrane protein segments using a combination of hydrophobicity and nonpolar phase helicity scales. Protein Sci. 2001 January; 10(1):212-9. doi: 10.1110/ps.30301. PMID: 11266608; PMCID: PMC2249854, which is hereby incorporated by reference in its entirety. In determining the helicity, the amino acid “X” can be any amino acid and all three instances of X in SEQ ID NO: 16 are the same amino acid. Thus, for example, if X were to be a methionine (M), then the sequence above would read KKAAAMAAAAAMAAWAAMAAAKKKK (SEQ ID NO: 17). The same holds true for every amino acid in Table 2 below, with the exception of cysteine (C). In determining the helicity of cysteine, Deber and colleagues provided a cysteine for the middle X position and utilized leucine (L) residues for the other two X positions. Thus, in determining the helicity of cysteine, the sequence above would read KKAAALAAAAACAAWAALAAAKKKK (SEQ ID NO: 18). Helicity values for each amino acid are in Table 2 below:

TABLE 2

| Amino Acid | Helicity |
|---|---|
| F | 1.26 |
| W | 1.07 |
| L | 1.28 |
| I | 1.29 |
| M | 1.22 |
| V | 1.27 |
| C | 0.79 |
| Y | 1.11 |
| A | 1.24 |
| T | 1.09 |
| E | 0.85 |
| D | 0.89 |
| Q | 0.96 |
| R | 0.95 |
| S | 1.00 |
| G | 1.15 |
| N | 0.94 |
| H | 0.97 |
| P | 0.57 |
| K | 0.88 |

A payload protein secreted by the various genetically modified bacteria disclosed herein, which are interchangeably referred to as “engineered bacteria”, may be provided to a subject in a pharmaceutical composition. Additionally or alternatively, the engineered bacteria itself may be provided to a subject in a pharmaceutical composition.

The various compositions disclosed herein may be useful in treating a number of diseases, for example, cancer. As used herein, cancer refers to a condition characterized by unregulated cell growth. Examples of cancer include, but are not limited to, squamous cell cancer, small-cell lung cancer, non-small cell lung cancer, lung adenocarcinoma, lung squamous cell carcinoma, gastrointestinal cancer, Hodgkin's and non-Hodgkin's lymphoma, pancreatic cancer, glioblastoma, cervical cancer, colon cancer, colorectal cancer, endometrial or uterine carcinoma, kidney cancer such as renal cell carcinoma and Wilms' tumors, basal cell carcinoma, melanoma, prostate cancer, and esophageal cancer.

The various compositions disclosed herein may comprise one or more drugs, biologics, or active agents, which are used interchangeably herein and refer to a chemical substance or compound that induces a desired pharmacological or physiological effect, and includes agents that are therapeutically effective, prophylactically effective, or cosmetically effective, i.e. the payload. “Drug,” “biologic,” and “active agent” include any pharmaceutically acceptable, pharmacologically active derivatives and analogs of those drugs, biologics, and active agents specifically mentioned herein, including, but not limited to, salts, esters, amides, prodrugs, active metabolites, inclusion complexes, analogs, and the like. Suitable drugs, biologics, and active agents may include, but are not limited to, alcohol deterrents; amino acids; ammonia detoxicants; anabolic agents; analeptic agents; analgesic agents; androgenic agents; anesthetic agents; anorectic compounds; anorexic agents; antagonists; anti-allergic agents; anti-amebic agents; anti-anemic agents; anti-anginal agents; anti-anxiety agents; anti-arthritic agents; anti-atherosclerotic agents; anti-bacterial agents; anti-cancer agents, including antineoplastic drugs, and anti-cancer supplementary potentiating agents; anticholinergics; anticholelithogenic agents; anti-coagulants; anti-coccidal agents; anti-convulsants; anti-depressants; anti-diabetic agents; anti-diarrheals; anti-diuretics; antidotes; anti-dyskinetics agents; anti-emetic agents; anti-epileptic agents; anti-estrogen agents; anti-fibrinolytic agents; anti-fungal agents; antiglaucoma agents; anti-hemophilic agents; anti-hemorrhagic agents; antihistamines; anti-hyperlipidemic agents; anti-hyperlipoproteinemic agents; antihypertensive agents; anti-hypotensives; anti-infective agents such as antibiotics and antiviral agents; anti-inflammatory agents, both steroidal and non-steroidal; anti-keratinizing agents; anti-malarial agents; antimicrobial agents; anti-migraine agents; anti-mitotic agents; anti-mycotic agents; antinauseants; antineoplastic agents; anti-neutropenic agents; anti-obsessional agents; anti-parasitic agents; antiparkinsonism drugs; anti-pneumocystic agents; anti-proliferative agents; anti-prostatic hypertrophy drugs; anti-protozoal agents; antipruritics; anti-psoriatic agents; antipsychotics; antipyretics; antispasmodics; anti-rheumatic agents; anti-schistosomal agents; anti-seborrheic agents; anti-spasmodic agents; anti-thrombotic agents; anti-tubercular agents; antitussive agents; anti-ulcerative agents; anti-urolithic agents; antiviral agents; GERD medications, anxiolytics; appetite suppressants; attention deficit disorder (ADD) and attention deficit hyperactivity disorder (ADHD) drugs; bacteriostatic and bactericidal agents; benign prostatic hyperplasia therapy agents; blood glucose regulators; bone resorption inhibitors; bronchodilators; carbonic anhydrase inhibitors; cardiovascular preparations including anti-anginal agents, anti-arrhythmic agents, beta-blockers, calcium channel blockers, cardiac depressants, cardiovascular agents, cardioprotectants, and cardiotonic agents; central nervous system (CNS) agents; central nervous system stimulants; choleretic agents; cholinergic agents; cholinergic agonists; cholinesterase deactivators; coccidiostat agents; cognition adjuvants and cognition enhancers; cough and cold preparations, including decongestants; depressants; diagnostic aids; diuretics; dopaminergic agents; ectoparasiticides; emetic agents; enzymes which inhibit the formation of plaque, calculus or dental caries; enzyme inhibitors; estrogens; fibrinolytic agents; fluoride anticavity/antidecay agents; free oxygen radical scavengers; gastrointestinal motility agents; genetic materials; glucocorticoids; gonad-stimulating principles; hemostatic agents; herbal remedies; histamine H2 receptor antagonists; hormones; hormonolytics; hypnotics; hypocholesterolemic agents; hypoglycemic agents; hypolipidemic agents; hypotensive agents; immunizing agents; immunomodulators; immunoregulators; immunostimulants; immunosuppressants; impotence therapy adjuncts; inhibitors; keratolytic agents; leukotriene inhibitors; liver disorder treatments; metal chelators such as ethylenediaminetetraacetic acid, tetrasodium salt; mitotic inhibitors; mood regulators; mucolytics; mucosal protective agents; muscle relaxants; mydriatic agents; narcotic antagonists; neuroleptic agents; neuromuscular blocking agents; neuroprotective agents; nicotine; NMDA antagonists; non-hormonal sterol derivatives; nutritional agents, such as vitamins, essential amino acids and fatty acids; ophthalmic drugs such as antiglaucoma agents; oxytocic agents; pain relieving agents; parasympatholytics; peptide drugs; plasminogen activators; platelet activating factor antagonists; platelet aggregation inhibitors; post-stroke and post-head trauma treatments; potentiators; progestins; prostaglandins; prostate growth inhibitors; proteolytic enzymes as wound cleansing agents; prothyrotropin agents; psychostimulants; psychotropic agents; radioactive agents; regulators; relaxants; repartitioning agents; scabicides; sclerosing agents; sedatives; sedative-hypnotic agents; selective adenosine A₁ antagonists; serotonin antagonists; serotonin inhibitors; serotonin receptor antagonists; steroids, including progestogens, estrogens, corticosteroids, androgens and anabolic agents; smoking cessation agents; stimulants; suppressants; sympathomimetics; synergists; thyroid hormones; thyroid inhibitors; thyromimetic agents; tranquilizers; tooth desensitizing agents; tooth whitening agents such as peroxides, metal chlorites, perborates, percarbonates, peroxyacids, and combinations thereof; unstable angina agents; uricosuric agents; vasoconstrictors; vasodilators including general coronary, peripheral and cerebral; vulnerary agents; wound healing agents; xanthine oxidase inhibitors; and the like.

The various compositions disclosed herein may comprise an effective amount of a drug, biologic, or active agent. Effective amount refers to an amount of a drug, biologic, or active agent (alone or with one or more other active agents) sufficient to induce a desired response, such as to prevent, treat, reduce and/or ameliorate a condition. An effective amount of an active agent, alone or with one or more other active agents, can be determined in many different ways, such as assaying for a reduction in of one or more signs or symptoms associated with the condition in the subject or measuring the level of one or more molecules associated with the condition to be treated.

The various compositions disclosed herein may alternatively comprise an effective amount of an agricultural product (e.g., pesticide, bactericide herbicide, fungicide, nematicide, miticide, plant growth regulator, plant growth stimulator, or fertilizer), a vaccine, a diagnostic protein, a feed conversion enzyme, a flavoring, or a nutritional protein. Effective amount refers to an amount of a product sufficient to induce a desired response, such as to prevent, enhance, treat, reduce and/or ameliorate a condition (e.g., promote growth, reduce insects, reduce weeds). As used herein, the phrase “agricultural setting” or “agricultural environment” or any synonymous phrase thereof is to be understood as any individual component or combination of components that make up the “setting” or “environment”. As such, “agricultural setting” in the context of the present disclosure can refer to a plant, a population of plants, the soil that a plant is grown in, a seed, a population of seeds, or any combination thereof. Additionally, the “agricultural setting” is not to be construed as being limited in size. Thus, in some embodiments. “agricultural setting” can refer to a seed, a batch of seeds, a single plant, a batch of plants, a field of plants, multiple fields of plants, the soil prepared for a single plant, a field in which plants can grow, multiple fields in which plants can grow, etc. Further, the type of plant is not meant to be limited in any way. Thus, while the “agricultural setting” may comprise plants such as produce crops (e.g. corn, cotton, fruit, tree nuts, rice, soybean and oil crops, sugar and sweeteners, vegetables, pulses, and wheat), the embodiments are not limited to produce crops. Rather, any plant that may benefit from the embodiments provided herein is also understood to fall under the term “agricultural setting”. The preceding examples of “agricultural setting” are not meant to be exhaustive or limiting in any way. One of skill in the art will understand that additional plants and plant cultivating systems and or environments also falls within the scope of the present disclosure as being an “agricultural setting”.

### Synthetic Pre-Protein Signal Peptides

Synthetic pre-protein signal peptides that increase secretion of a payload protein from bacteria are provided herein. Table 3 below lists various amino acid and polynucleotide sequences that will be referred to herein.

TABLE 3

| SEQ ID | | |
|---|---|---|
| NO: | Sequence | Description |
| 1 | MKKLLALGLLALGLLLSSSAQAAD | Pre-Protein Signal |
| | | Peptide |
| 2 | MKKLLASLVLALLLLASSSAFAAD | Pre-Protein Signal |
| | | Peptide |
| 3 | MKKNLLLASLLLLLALASGSAQAAD | Pre-Protein Signal |
| | | Peptide |
| 4 | MKKIKLLLLLLLLALLLSGSAFAAD | Pre-Protein Signal |
| | | Peptide |
| 5 | atg aaa aaa ctg ctg gcg ctg ggc ctg ctg gcg ctg ggc ctg | Polynucleotide encoding |
| | ctg ctg agc agc agc gcg cag gcg gcg gat | SEQ ID NO: 1 |
| 6 | atg aaa aaa ctg ctg gcg agc ctg gtg ctg gcg ctg ctg ctg | Polynucleotide encoding |
| | ctg gcg agc agc agc gcg ttt gcg gcg gat | SEQ ID NO: 2 |
| 7 | atg aaa aaa aac ctg ctg ctg gcg agc ctg ctg ctg ctg ctg | Polynucleotide encoding |
| | gcg ctg gcg agc ggc agc gcg cag gcg gcg gat | SEQ ID NO: 3 |
| 8 | atg aaa aaa att aaa ctg ctg ctg ctg ctg ctg ctg ctg gcg | Polynucleotide encoding |
| | ctg ctg ctg agc ggc agc gcg ttt gcg gcg gat | SEQ ID NO: 4 |
| 9 | LEISS TCDA | Peptide Spacer |
| 10 | ttg gaa atc tcc tca aca tgc gat gcc | Polynucleotide encoding |
| | | SEQ ID NO: 9 |
| 11 | FCTFF EKHHR KWDIL LEKST GVMEA | MISTIC |
| 12 | ttc tgt act ttt ttc gag aaa cat cac cgg aag tgg gac ata | Polynucleotide encoding |
| | ctt tta gag aaa tcc act ggg gtg atg gaa gcg | MISTIC |
| 13 | MFAKR FKTSL LPLFA GFLLL FHLVL AGPAA | Signal Peptide AmyE |
| | ASAET A | (Type I/Sec) |
| 14 | MVSIR RSFEA YVDDM NIITV LIPAE QKEIM | Signal Peptide AmyX |
| | | (Type II/Tat) |
| 15 | MRSKKLWISLLFALTLIFTMAFSNMSAQA | AprE pre-protein signal |
| | | peptide |
| 66 | MKYLLPTAAAGLLLLAAQPAMA | PelB signal peptide |
| 67 | atg aaa tat ctg ctg ccg act geg gca geg ggt tta ctg ctg | Polynucleotide encoding |
| | ctg gca geg caa ccg gcc atg gcc | SEQ ID NO: 66 |
| 68 | MKKTAIAIAVALAGFATVAQA | OmpA signal peptide |
| 69 | atg aaa aaa acc geg atc gog atc gcg gtt gcg ctg gcg ggt | Polynucleotide encoding |
| | ttc geg acc gtt gcg cag gcg | SEQ ID NO: 68 |
| 70 | MKIKTGARILALSALTTMMFSASALA | MalE signal peptide |
| 71 | atg aaa ata aaa aca ggt gca cgc atc ctc gca tta tcc gca tta | Polynucleotide encoding |
| | acg acg atg atg ttt tcc gcc teg gct ctc gcc | SEQ ID NO: 70 |

The synthetic pre-protein signal peptides disclosed herein are optimized for use in Escherichia bacteria and can be used to induce expression of any protein within the Escherichia genus. Particular examples of suitable bacteria species are provided herein below to exemplify the particular synthetic signal peptides that have been developed.

As noted above, Table 3 discloses amino acid and polynucleotide sequences. However, in any aspect and embodiment, SEQ ID NO: 1, SEQ ID NO: 2, SEQ ID NO: 3, and SEQ ID NO: 4 in Table 3 may be modified with conservative amino acid substitutions to produce active variants that maintain the characteristics and functionality of the primary sequence.

For example, in any embodiment, one or more of the leucine (L) residues in SEQ ID NO: 1 may be independently substituted with at least A, V, F, or I, SEQ ID NO: 2 includes two adjacent lysine (K) residues. In any embodiment, one or more of the lysine (K) residues in SEQ ID NO: 2 may be substituted with at least an arginine (R). Optionally, the two K residues may be substituted with a single K residue or with three K residues, each residue of which may be optionally substituted with at least an arginine (R). In any embodiment, one or more of the alanine (A) residues may be independently substituted with at least V, N, T, or G. In any embodiment, the c-terminal aspartate (D) residue may be substituted with at least V, N, T, or G. In any embodiment, any glycine (G) residue may be substituted with at least S, N, or Q. In any embodiment, any serine(S) residue may be independently substituted with at least N, T, G, or V. Any of the aforementioned substitutions may be combined to make two or more types of conservative amino substitutions. For example, in any embodiment, one or more of the leucine (L) residues in SEQ ID NO: 1 may be independently substituted with at least A, V, F, or I and one or more of the alanine (A) residues may be independently substituted with at least V, N, T, or G. In another example, the c-terminal D residue may be substituted with at least V, N, T, or G and any (G) residue may be substituted with at least S, N, or Q.

Such conservative amino acid substitutions also apply to SEQ ID NO: 3 and SEQ ID NO: 4. The previous examples of conservative amino acid substitutions were for illustrative purposes only, and not meant to be limiting in any way. One of skill in the art will be able to envisage various conservative amino acid substitutions that correspond to the various embodiments disclosed above. However, these conservative amino acid substitutions can be generally described by the Formula below, which encapsulate the parent sequence as well as the variant sequences. The Formula detailing the variant sequences will now be described.

### Variants of SEQ ID NO: 1-4 (Formula I)

In some embodiments, the pre-protein signal peptide comprises an amino acid sequence represented by (“Formula I”):

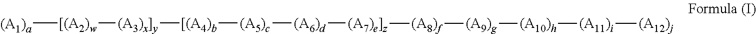

wherein A₂-A₁₂ have one or more of the properties described in Table 4 below:

TABLE 4

| | Isoelectric | Molecular | | |
|---|---|---|---|---|
| AA Label | Point | Weight | HP Index | Helicity |
| A₂ | 5.4-10.8 | 89-175 | −4-31 | 0.5-1.3 |
| A₃ | 5.4-7.7 | 117-182 | −5.1-31 | 0.9-1.3 |
| A₄ | 5-10.8 | 75-205 | −5.1-34 | 0.5-1.3 |
| A₅ | 2.7-10.8 | 75-205 | −5.1-34 | 0.5-1.3 |
| A₆ | 5.4-6 | 75-205 | 0-0.7 | 0.9-1.2 |
| A₇ | 5-6.4 | 75-147 | 0-31 | 0.5-1.3 |
| A₈, A₁₀, A₁₁ | 2.7-10.8 | 75-205 | −5.1-34 | 0.5-1.3 |
| A₉ | 2.7-10.8 | 75-205 | −5.1-34 | 0.5-1.3 |
| A₁₂ | 2.7-10.8 | 75-205 | −5.1-34 | 0.5-1.3 |

wherein each w is, independently, an integer selected from 1-2 (inclusive;
   each x is, independently, an integer selected from 0-1 (inclusive);
   y is an integer selected from 1-3 (inclusive);
   z is an integer selected from 2-12 (inclusive);
   each b, c, d, and e are each, independently, an integer selected from 0 or 1 (inclusive); and
   a, f, g, h, i, and j are each, independently, an integer selected form 0 or 1 (inclusive).

In some embodiments, w may be any integer between 1 and 2. In some embodiments, wis 1. In some embodiments, w is 2. In some embodiments, x is 0. In some embodiments, x is 1. In some embodiments, y is 1. In some embodiments, y is 2. In some embodiments, y is 3. In some embodiments, z is 2. In some embodiments, z is 3. In some embodiments, z is 4. In some embodiments, z is 5. In some embodiments, z is 6. In some embodiments, z is 7. In some embodiments, z is 8. In some embodiments, z is 9. In some embodiments, z is 10. In some embodiments, z is 11. In some embodiments, z is 12. In some embodiments, a is 0. In some embodiments, a is 1. In some embodiments, b is 0. In some embodiments, b is 1. In some embodiments, c is 0). In some embodiments, c is 1. In some embodiments, d is 0). In some embodiments, d is 1. In some embodiments, e is 0. In some embodiments, e is 1. In some embodiments, f is 0. In some embodiments, f is 1. In some embodiments, g is 0. In some embodiments, g is 1. In some embodiments, h is 0. In some embodiments, h is 1. In some embodiments, i is 0. In some embodiments, i is 1. In some embodiments, j is 0. In some embodiments, j is 1. It is to be understood that the values of q, w, x, y, and z are each independently selected, and the values of any variable w, x, y, z, or a is independent of the values selected for any of the other variables. Further, in embodiments where w, y, and z are values greater than 1, each amino acid described in that group may be selected from the disclosed list independently of other. For example, in embodiments where w is 2 and y is 1, the 2 amino acids described by A₂ may each independently be K, R, N, A, P, S, T, I, or F. Both may be the same, or each may be a different amino acid. For example, the sequence represented by (A₂)₂ where w is 2 and y is I may be KK, KR, KN, KA, KP, KS, KT, KI, KF, RK, RR, RN, RA, and so on. This meaning, unless explicitly indicated otherwise, expands to all further formulas disclosed herein and below.

In some embodiments, A₁ is absent. In some embodiments, A₁ is present and is methionine (M). In some embodiments, each A₂ is, independently, an amino acid having an isoelectric point of about 5.4 to about 10.8, a molecular weight of about 89 g/mol to about 175 g/mol, a hydropathy index of about −4 to about 31, and a helicity of about 0.5 to about 1.3. In some embodiments, each A₃ is, independently, absent. In some embodiments, each A₃ is, independently, an amino acid having an isoelectric point of about 5.4 to about 7.7, a molecular weight of about 117 g/mol to about 182 g/mol, a hydropathy index of about −5.1 to about 31, and a helicity of about 0.9 to about 1.3. In some embodiments, each A₄ is, independently, absent. In some embodiments, each A₄ is, independently, an amino acid having an isoelectric point of about 5 to about 10.8, a molecular weight of about 75 g/mol to about 205 g/mol, a hydropathy index of about −5.1 to about 34, and a helicity of about 0.5 to about 1.3. In some embodiments, each A₅ is, independently, absent. In some embodiments, each A₅ is, independently, an amino acid having an isoelectric point of about 2.7 to about 10.8, a molecular weight of about 75 g/mol to about 205 g/mol, a hydropathy index of about −5.1 to about 34, and a helicity of about 0.5 to about 1.3. In some embodiments, each A₆ is, independently, absent. In some embodiments, each A₆ is, independently, an amino acid having an isoelectric point of about 5.4 to about 6, a molecular weight of about 75 g/mol to about 205 g/mol, a hydropathy index of about 0) to about 0.7, and a helicity of about 0.9 to about 1.2. In some embodiments, each A₇ is, independently, absent. In some embodiments, each A₇ is, independently, an amino acid having an isoelectric point of about 5 to about 6.4, a molecular weight of about 75 g/mol to about 147 g/mol, a hydropathy index of about 0 to about 31, and a helicity of about 0.5 to about 1.3. In some embodiments, each A₈ is, independently, absent. In some embodiments, each A₈ is, independently, an amino acid having an isoelectric point of about 2.7 to about 10.8, a molecular weight of about 75 g/mol to about 205 g/mol, a hydropathy index of about −5.1 to about 34, and a helicity of about 0.5 to about 1.3. In some embodiments, each A₉ is, independently, absent. In some embodiments, each A₉ is, independently, an amino acid having an isoelectric point of about 2.7 to about 10.8, a molecular weight of about 75 g/mol to about 205 g/mol, a hydropathy index of about −5.1 to about 34, and a helicity of about 0.5 to about 1.3. In some embodiments, each A₁₀ is, independently, absent. In some embodiments, each A₁₀ is, independently, an amino acid having an isoelectric point of about 2.7 to about 10.8, a molecular weight of about 75 g/mol to about 205 g/mol, a hydropathy index of about −5.1 to about 34, and a helicity of about 0.5 to about 1.3. In some embodiments, each A₁₁ is, independently, absent. In some embodiments, each A₁₁ is, independently, an amino acid having an isoelectric point of about 2.7 to about 10.8, a molecular weight of about 75 g/mol to about 205 g/mol, a hydropathy index of about −5.1 to about 34, and a helicity of about 0.5 to about 1.3. In some embodiments, each A₁₂ is, independently, absent. In some embodiments, each A₁₂ is, independently, an amino acid having an isoelectric point of about 2.7 to about 10.8, a molecular weight of about 75 g/mol to about 205 g/mol, a hydropathy index of about −5.1 to about 34, and a helicity of about 0.5 to about 1.3.

In some embodiments, A₁ is absent. In some embodiments, A₁ is present and is methionine (M). In some embodiments, each A₂ is, independently, an amino acid selected from the group consisting of K, R, N, A, P, S, T, I, and F. In some embodiments, each A₂ is, independently, selected from the group consisting of K, R, and N. In some embodiments, each A₃ is, independently, absent. In some embodiments, each A₃ is, independently, an amino acid selected from the group consisting of I, L, F, V, M, Y, and H. In some embodiments, each A₃ is, independently, isoleucine (I). In some embodiments, each A₄ is, independently, absent. In some embodiments, each A₄ is, independently, an amino acid selected from the group consisting of L, V, C, A, F, I, T, M, P, S, G, W, Y, Q, N, R, and H. In some embodiments, each A₄ is, independently, an amino acid selected from the group consisting of L, V, C, and A. In some embodiments, each A₅ is, independently, absent. In some embodiments, each A₅ is, independently, an amino acid selected from the group consisting of A, G, S, T, P, M, C, V, W, I, L, F, Y, Q, N, R, E, K, D, and H. In some embodiments, each A₅ is, independently, an amino acid selected from the group consisting of A, G, and S. In some embodiments, each A₆ is, independently, absent. In some embodiments, each A₆ is, independently, an amino acid selected from the group consisting of G, S, N, and Q. In some embodiments, each A₇ is, independently, absent. In some embodiments, each A₇ is, independently, an amino acid selected from the group consisting of S, N, T, G, V, I, Q, A, C, P, Y, M, F, and L. In some embodiments, each A₇ is, independently, an amino acid selected from the group consisting of S, N, T, G, V, and I. In some embodiments, A₈ is absent. In some embodiments A₈ is an amino acid selected from the group consisting of A, T, G, S, P, V, I, L, Q, R, M, W, F, C, N, K, E, D, and H. In some embodiments, A₈ is an amino acid selected from the group consisting of A, T, G, and S. In some embodiments, A₉ is absent. In some embodiments, A₉ is an amino acid selected from the group consisting of Q, F, N, S, E, T, D, R, H, K, G, A, P, Y, M, V, W, I, and L. In some embodiments, A₉ is an amino acid selected from the group consisting of Q and F. In some embodiments, A₁₀ is absent. In some embodiments A₁₀ is an amino acid selected from the group consisting of A, T, G, S, P, V, I, L, Q, R, M, W, F, C, N, K, E, D, and H. In some embodiments, A₁₀ is an amino acid selected from the group consisting of A, T, G, and S. In some embodiments, A₁₁ is absent. In some embodiments A₁₁ is an amino acid selected from the group consisting of A, T, G, S, P, V, I, L, Q, R, M, W, F, C, N, K, E, D, and H. In some embodiments, A₁₁ is an amino acid selected from the group consisting of A, T, G, and S. In some embodiments, A₁₂ is absent. In some embodiments, A₁₂ is an amino acid selected from the group consisting of D, E, Q, N, S, H, T, R, K, G, A, C, Y, P, M, V, W, I, and L. It is to be understood that unless explicitly stated the identity of each variable A₁-A₁₂ is independent of any other variable A₁-A₁₂. Thus, unless explicitly stated, the identity of A₁ does not affect the identity of A₂, the identity of A₁ does not affect the identity of A₄, the identity of A₃ does not affect the identity of A₈, and so forth.

Further, in consideration of [(A₂)_{w}-(A₃)ₓ]_{y}, it is to be understood that in embodiments where y is an integer greater than 1, the formula [(A₂)_{w}-(A₃)ₓ]_{y} does not indicate that [(A₂)_{w}-(A₃)ₓ] is repeated y number of times. Rather, when [(A₂)_{w}-(A₃)ₓ] is expanded due to a y greater than 1, each instance of A₂ can independently be selected from an appropriate amino acid as detailed above and likewise each instance of A₃ can independently be selected from an appropriate amino acid as detailed above. For example, in embodiments wherein w=1, x=1, and y=2, producing [(A₂)₁-(A₃)₁]₂, the formula could produce the sequence KIKI (SEQ ID NO: 19) wherein the first and second A₂ are both K, and the first and second A₃ are both I. The formula could also produce the sequence KIRL (SEQ ID NO: 20), wherein the first A₂ is K, the first A₃ is I, the second A₂ is R, and the second A₃ is L. In a further example, in embodiments wherein w=1, x=1, and y=3, producing [(A₂)₁-(A₃)₁]₃, the formula could produce the sequence KIKIKI (SEQ ID NO: 21) wherein the first, second, and third A₂ are all K, and the first, second, and third A₃ are all I. The formula could also produce the sequence KIRLNF (SEQ ID NO: 22), wherein the first A₂ is K, the first A₃ is I, the second A₂ is R, the second A₃ is L, the third A₂ is N, and the third A₃ is F. The same functionality of y also holds true for the values of w and x. For example, in embodiments wherein y=2, producing [(A₂)_{w}-(A₃)ₓ]₂, each instance of w may be an integer from 1-2, and each instance of x may be an integer from 0-1. Thus, in the context of the variable x, the first instance of x and the second instance of x may each be 0, the first instance of x and the second instance of x may each be 1, or the first instance of x may be 0 and the second instance of x may be 1.

Thus, for example, when considering the formula of Formula I (A₁)ₐ-[(A₂)_{w}-(A₃)ₓ]_{y}-[(A₄)_{b}-(A₅)_{c}-(A₆)_{d}-(A₇)ₑ]_{z}-(A₈)_{f}-(A₉)_{g}-(A₁₀)ₕ-(A₁₁)ₗ-(A₁₂)ⱼ wherein y is 3, one can also envision the formula of Formula I to be written as:

(A₁)ₐ-(A₂)_{w}-(A₃)ₓ-(A₂)_{w}-(A₃)ₓ-(A₂)_{w}-(A₃)ₓ-[(A₄)_{b}-(A₅)_{c}-(A₆)_{d}-(A₇)ₑ]_{z}-(A₈)_{f}-(A₉)_{g}-(A₁₀)ₕ-(A₁₁)ᵢ-(A₁₂)ⱼ
wherein each w is independently selected from 1 or 2, each x is independently selected from 0 or 1, and each A₂ and A₃ is each selected, independently, from an appropriate amino acid as outlined above. This meaning, unless explicitly indicated otherwise, expands to all further formulas disclosed herein and below.

In some embodiments, the identity of each instance of each position A₁ through A₁₂ is independently selected from Table 5 below:

TABLE 5

| Position | Amino acid |
|---|---|
| A₁ | Absent or methionine (M) |
| A₂ | K, R, N, A, P, S, T, I, or F |
| A₃ | Absent or I, L, F, V, M, Y, or H |
| A₄ | Absent or L, V, C, A, F, I, T, M, P, S, G, W, Y, Q, N, R, or H |
| A₅ | Absent or A, G, S, T, P, M, C, V, W, I, L, F, Y, Q, N, R, E, K, D, and H |
| A₆ | Absent or G, S, N, or Q |
| A₇ | Absent or S, N, T, G, V, I, Q, A, C, P, Y, M, F, or L |
| A₈ | Absent or A, T, G, S, P, V, I, L, Q, R, M, W, F, C, N, K, E, D, or H |
| A₉ | Absent or Q, F, N, S, E, T, D, R, H, K, G, A, P, Y, M, V, W, I, or L |
| A₁₀ | Absent or A, T, G, S, P, V, I, L, Q, R, M, W, F, C, N, K, E, D, or H |
| A₁₁ | Absent or A, T, G, S, P, V, I, L, Q, R, M, W, F, C, N, K, E, D, or H |
| A₁₂ | Absent or D, E, Q, N, S, H, T, R, K, G, A, C, Y, P, M, V, W, I, or L |

In some embodiments, the identity of each instance of each position A₁ through A₁₂ is independently selected from Table 6 below:

TABLE 6

| Position | Amino acid |
|---|---|
| A₁ | Absent or methionine (M) |
| A₂ | K, R, or N |
| A₃ | Absent or isoleucine (I) |
| A₄ | Absent or L, V, C, or A |
| A₅ | Absent or A, G, or S |
| A₆ | Absent or G, S, N, or Q |
| A₇ | Absent or S, N, T, G, V, or I |
| A₈ | Absent or A, T, G, or S |
| A₉ | Absent or Q, or F |
| A₁₀ | Absent or A, T, G, or S |
| A₁₁ | Absent or A, T, G, or S |
| A₁₂ | Absent or D, E, Q, N, S, H, T, R, |
| | K, G, A, C, Y, P, M, V, W, I, or L |

In some embodiments, the identity of each instance of each position A₁ through A₁₂ is independently selected from Table 5. In some embodiments, the identity of each instance of each position A₁ through A₁₂ is independently selected from Table 6. In some embodiments, the identity of each instance of each position A₁ through A₁₂ is independently selected from a combination of Table 5 and Table 6. For example, positions A₁-A₆ may be selected from the embodiments provided in Table 5, where positions A₇-A₁₂ may be selected from the embodiments provided in Table 6. Alternatively, positions A₁, A₃-A₅ and A₁₂ may be selected from the embodiments provided in Table 5, where positions A₂ and A₆-A₁₁ may be selected from the embodiments provided in Table 6. The preceding two embodiments are exemplary only, and not meant to be limiting in any way. Each position may, independently, be selected from Table 5 or Table 6. Accordingly, in some embodiments, A₁ has an identity selected from Table 5 or Table 6; each A₂ has, independently, an identity selected from Table 5 or Table 6; each A₃ has, independently, an identity selected from Table 5 or Table 6; each A₄ has, independently, an identity selected from Table 5 or Table 6; each A₅ has, independently, an identity selected from Table 5 or Table 6; each A₆ has, independently, an identity selected from Table 5 or Table 6; each A₇ has, independently, an identity selected from Table 5 or Table 6; A₈ has an identity selected from Table 5 or Table 6; A₉ has an identity selected from Table 5 or Table 6; A₁₀ has an identity selected from Table 5 or Table 6; A₁₁ has an identity selected from Table 5 or Table 6; and A₁₂ has an identity selected from Table 5 or Table 6.

In some embodiments, a pre-protein signal peptide sequence generated by the formula of Formula I can be any length that is within the parameters of the formula. In some embodiments, a pre-protein signal peptide sequence generated by the formula of Formula I comprises a minimum number of amino acids, a maximum number of amino acids, or both. In some embodiments, a pre-protein signal peptide sequence generated by the formula of Formula I comprises a minimum of 15 amino acids. In some embodiments, a pre-protein signal peptide sequence generated by the formula of Formula I comprises a maximum of 45 amino acids. In some embodiments, a pre-protein signal peptide sequence generated by the formula of Formula I comprises a minimum of 15 amino acids and a maximum of 45 amino acids. In some embodiments, the pre-protein signal peptide comprises an amino acid sequence of Formula I, wherein Formula I is represented as:

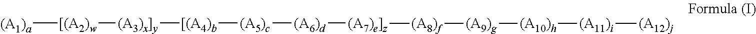

wherein:
   each w is, independently, 1 or 2;
   each x is, independently, 0 or 1;
   y is 1, 2, or 3;
   z is 2, 3, 4, 5, 6, 7, 8, 9, 10, 11, or 12;
   each b, c, d, and e are each, independently, 0 or 1,
   a, f, g, h, i, and j are each, independently, 0 or 1;
   and the pre-protein signal peptide comprises a minimum length of 15 amino acids.

In some embodiments, the pre-protein signal peptide comprises an amino acid sequence of Formula I, wherein Formula I is represented as:

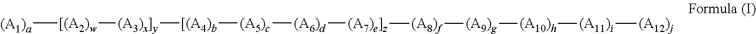

wherein:
   each w is, independently, 1 or 2;
   each x is, independently, 0 or 1;
   y is 1, 2, or 3;
   z is 2, 3, 4, 5, 6, 7, 8, 9, 10, 11, or 12;
   each b, c, d, and e are each, independently, 0 or 1,
   a, f, g, h, i, and j are each, independently, 0 or 1;
   and the pre-protein signal peptide comprises a maximum length of 45 amino acids.

In some embodiments, the pre-protein signal peptide comprises an amino acid sequence of Formula I, wherein Formula I is represented as:

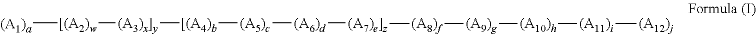

wherein:
   each w is, independently, 1 or 2;
   each x is, independently, 0 or 1;
   y is 1, 2, or 3;
   z is 2, 3, 4, 5, 6, 7, 8, 9, 10, 11, or 12;
   each b, c, d, and e are each, independently, 0 or 1,
   a, f, g, h, i, and j are each, independently, 0 or 1;
   and the pre-protein signal peptide comprises a minimum length of 15 amino acids and a maximum length of 45 amino acids.

In some embodiments, the sequence of SEQ ID NO: 1 can be derived from Formula I as follows: a is 1, y is 1, w is 2, x is 1, z is 5, all 5 instances of b are 1, all 5 instances of c are 1, all 5 instances of d are 0, all 5 instances of e are 1, f is 1, g is 1, h is 1, i is 1, and j is 1; A₁ is methionine (M); the string of three (3) amino acid residues as represented by [(A₂)₂-(A₃)₁]₁ are as follows: K-K-L; the string of fifteen (15) amino acid residues as represented by [(A₄)₁-(A₅)₁-(A₇)₁]₅ are as follows: LALGLLALGLLLSSS (SEQ ID NO: 23): A₆ is absent; A₈ is alanine (A); A₉ is glutamine (Q); A₁₀ is alanine (A); A₁₁ is alanine (A); and A₁₂ is aspartic acid (D).

In some embodiments, the sequence of SEQ ID NO: 2 can be derived from Formula I as follows: a is 1, y is 1, w is 2, x is 1, z is 5, all 5 instances of b are 1, all 5 instances of c are 1, all 5 instances of d are 0, all 5 instances of e are 1, f is 1, g is 1, h is 1, i is 1, and j is 1; A₁ is methionine (M); the string of three (3) amino acid residues as represented by [(A₂)₂-(A₃)₁]₁ are as follows: K-K-L; the string of fifteen (15) amino acid residues as represented by [(A₄)₁-(A₅)₁-(A₇)₁]₅ are as follows: LASLVLALLLLASSS (SEQ ID NO: 24); A₆ is absent; A₈ is alanine (A); A₉ is phenylalanine (F); A₁₀ is alanine (A); A₁₁ is alanine (A); and A₁₂ is aspartic acid (D).

In some embodiments, the sequence of SEQ ID NO: 3 can be derived from Formula I as follows: a is 1, y is 1, w is 2, x is 0, z is 6, all 6 instances of b are 1, all 6 instances of c are 1, all 6 instances of d are 0, all 6 instances of e are 1, f is 0, g is 1, h is 1, i is 1, and j is 1; A₁ is methionine (M); the string of two (2) amino acid residues as represented by [(A₂)₂]₁ are as follows: K-K; A₃ is absent; the string of eighteen (18) amino acid residues as represented by [(A₄)₁-(A₅)₁-(A₇)₁]₆ are as follows: NLLLASLLLLLALASGSA (SEQ ID NO: 25); A₆ is absent; A₈ is absent; A₉ is glutamine (Q); A₁₀ is alanine (A); A₁₁ is alanine (A); and A₁₂ is aspartic acid (D).

In some embodiments, the sequence of SEQ ID NO: 4 can be derived from Formula I as follows: a is 1, y is 2, the first instance of w is 2, the second instance of w is 1, the first and second instance of x is 1, z is 5, all 5 instances of b are 1, all 5 instances of c are 1, all 5 instances of d are 0, all 5 instances of e are 1, f is 0, g is 1, h is 1, i is 1, and j is 1; A₁ is methionine (M); the string of five (5) amino acid residues as represented by [(A₂)_{w}-(A₃)ₓ]_{y}, wherein y, w, and x are defined above and the formula is expanded to (A₂)-(A₂)-(A₃)-(A₂)-(A₃) as provided for herein are as follows: KKIKL (SEQ ID NO: 26); the string of fifteen (15) amino acid residues as represented by [(A₄)₁-(A₅)₁-(A₇)₁]₅ are as follows: LLLLLLLALLLSGSA (SEQ ID NO: 27); A₆ is absent; A₈ is absent; A₉ is phenylalanine (F); A₁₀ is alanine (A); A₁₁ is alanine (A); and A₁₂ is aspartic acid (D).

In some embodiments, a pre-protein signal peptide is provided. In some embodiments, the pre-protein signal peptide comprises an amino acid sequence of Formula I. In some embodiments, the pre-protein signal peptide comprising an amino acid sequence of Formula I increases the secretion of a payload protein appended thereto as compared to native signal peptides.

In some embodiments, the pre-protein signal peptide comprises an amino acid sequence having at last 70% identity to an amino acid sequence selected from the group consisting of SEQ ID NO: 1, 2, 3, or 4. In some embodiments, the pre-protein signal peptide comprises an amino acid sequence having at last 70% identity to SEQ ID NO: 1. In some embodiments, the pre-protein signal peptide comprises an amino acid sequence having at last 70% identity to SEQ ID NO: 2. In some embodiments, the pre-protein signal peptide comprises an amino acid sequence having at last 70% identity to SEQ ID NO: 3. In some embodiments, the pre-protein signal peptide comprises an amino acid sequence having at last 70% identity to SEQ ID NO: 4. In some embodiments, the pre-protein signal peptide comprising an amino acid sequence having at last 70% identity to an amino acid sequence selected from the group consisting of SEQ ID NO: 1, 2, 3, or 4 increases the secretion of a payload protein appended thereto as compared to native signal peptides.

In some embodiments, the pre-protein signal peptide comprises an amino acid sequence having at least at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to an amino acid sequence selected from the group consisting of SEQ ID NO: 1, 2, 3, or 4.

In some embodiments, the pre-protein signal peptide comprises an amino acid having at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to an amino acid sequence selected from the group consisting of SEQ ID NO: 1.

In some embodiments, the pre-protein signal peptide comprises an amino acid having at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to an amino acid sequence selected from the group consisting of SEQ ID NO: 2.

In some embodiments, the pre-protein signal peptide comprises an amino acid having at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to an amino acid sequence selected from the group consisting of SEQ ID NO: 3.

In some embodiments, the pre-protein signal peptide comprises an amino acid having at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to an amino acid sequence selected from the group consisting of SEQ ID NO: 4.

In some embodiments, the pre-protein signal peptide comprising an amino acid sequence having at last 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to an amino acid sequence selected from the group consisting of SEQ ID NO: 1, 2, 3, or 4 increases the secretion of a payload protein appended thereto as compared to native signal peptides.

In some embodiments, the pre-protein signal peptide comprises an amino acid sequence having at least 80% identity to an amino acid sequence selected from the group consisting of SEQ ID NO: 1, 2, 3, or 4. In some embodiments, the pre-protein signal peptide comprises an amino acid sequence having at least 80% identity to SEQ ID NO: 1. In some embodiments, the pre-protein signal peptide comprises an amino acid sequence having at least 80% identity to SEQ ID NO: 2. In some embodiments, the pre-protein signal peptide comprises an amino acid sequence having at least 80% identity to SEQ ID NO: 3. In some embodiments, the pre-protein signal peptide comprises an amino acid sequence having at least 80% identity to SEQ ID NO: 4. In some embodiments, the pre-protein signal peptide comprising an amino acid sequence having at least 80% identity to an amino acid sequence selected from the group consisting of SEQ ID NO: 1, 2, 3, or 4 increases the secretion of a payload protein appended thereto as compared to native signal peptides.

In some embodiments, the pre-protein signal peptide comprises an amino acid sequence having at least 85% identity to an amino acid sequence selected from the group consisting of SEQ ID NO: 1, 2, 3, or 4. In some embodiments, the pre-protein signal peptide comprises an amino acid sequence having at least 85% identity to SEQ ID NO: 1. In some embodiments, the pre-protein signal peptide comprises an amino acid sequence having at least 85% identity to SEQ ID NO: 2. In some embodiments, the pre-protein signal peptide comprises an amino acid sequence having at least 85% identity to SEQ ID NO: 3. In some embodiments, the pre-protein signal peptide comprises an amino acid sequence having at least 85% identity to SEQ ID NO: 4. In some embodiments, the pre-protein signal peptide comprising an amino acid sequence having at least 85% identity to an amino acid sequence selected from the group consisting of SEQ ID NO: 1, 2, 3, or 4 increases the secretion of a payload protein appended thereto as compared to native signal peptides.

In some embodiments, the pre-protein signal peptide comprises an amino acid sequence having at least 90% identity to an amino acid sequence selected from the group consisting of SEQ ID NO: 1, 2, 3, or 4. In some embodiments, the pre-protein signal peptide comprises an amino acid sequence having at least 90% identity to SEQ ID NO: 1. In some embodiments, the pre-protein signal peptide comprises an amino acid sequence having at least 90% identity to SEQ ID NO: 2. In some embodiments, the pre-protein signal peptide comprises an amino acid sequence having at least 90% identity to SEQ ID NO: 3. In some embodiments, the pre-protein signal peptide comprises an amino acid sequence having at least 90% identity to SEQ ID NO: 4. In some embodiments, the pre-protein signal peptide comprising an amino acid sequence having at least 90% identity to an amino acid sequence selected from the group consisting of SEQ ID NO: 1, 2, 3, or 4 increases the secretion of a payload protein appended thereto as compared to native signal peptides.

In some embodiments, the pre-protein signal peptide comprises an amino acid sequence having at least 95% identity to an amino acid sequence selected from the group consisting of SEQ ID NO: 1, 2, 3, or 4. In some embodiments, the pre-protein signal peptide comprises an amino acid sequence having at least 95% identity to SEQ ID NO: 1. In some embodiments, the pre-protein signal peptide comprises an amino acid sequence having at least 95% identity to SEQ ID NO: 2. In some embodiments, the pre-protein signal peptide comprises an amino acid sequence having at least 95% identity to SEQ ID NO: 3. In some embodiments, the pre-protein signal peptide comprises an amino acid sequence having at least 95% identity to SEQ ID NO: 4. In some embodiments, the pre-protein signal peptide comprising an amino acid sequence having at least 95% identity to an amino acid sequence selected from the group consisting of SEQ ID NO: 1, 2, 3, or 4 increases the secretion of a payload protein appended thereto as compared to native signal peptides.

In some embodiments, the pre-protein signal peptide comprises an amino acid sequence having at least 98% identity to an amino acid sequence selected from the group consisting of SEQ ID NO: 1, 2, 3, or 4. In some embodiments, the pre-protein signal peptide comprises an amino acid sequence having at least 98% identity to SEQ ID NO: 1. In some embodiments, the pre-protein signal peptide comprises an amino acid sequence having at least 98% identity to SEQ ID NO: 2. In some embodiments, the pre-protein signal peptide comprises an amino acid sequence having at least 98% identity to SEQ ID NO: 3. In some embodiments, the pre-protein signal peptide comprises an amino acid sequence having at least 98% identity to SEQ ID NO: 4. In some embodiments, the pre-protein signal peptide comprising an amino acid sequence having at least 98% identity to an amino acid sequence selected from the group consisting of SEQ ID NO: 1, 2, 3, or 4 increases the secretion of a payload protein appended thereto as compared to native signal peptides.

In some embodiments, the pre-protein signal peptide comprises an amino acid sequence selected from the group consisting of SEQ ID NO: 1, 2, 3, or 4. In some embodiments, the pre-protein signal peptide comprises an amino acid sequence of SEQ ID NO: 1. In some embodiments, the pre-protein signal peptide comprises an amino acid sequence of SEQ ID NO: 2. In some embodiments, the pre-protein signal peptide comprises an amino acid sequence of SEQ ID NO: 3. In some embodiments, the pre-protein signal peptide comprises an amino acid sequence of SEQ ID NO: 4. In some embodiments, the pre-protein signal peptide comprising an amino acid sequence selected from the group consisting of SEQ ID NO: 1, 2, 3, or 4 increases the secretion of a payload protein appended thereto as compared to native signal peptides. In some embodiments, the pre-protein signal peptide comprising an amino acid sequence of SEQ ID NO: 1 increases the secretion of a payload protein appended thereto as compared to native signal peptides. In some embodiments, the pre-protein signal peptide comprising an amino acid sequence of SEQ ID NO: 2 increases the secretion of a payload protein appended thereto as compared to native signal peptides. In some embodiments, the pre-protein signal peptide comprising an amino acid sequence of SEQ ID NO: 3 increases the secretion of a payload protein appended thereto as compared to native signal peptides. In some embodiments, the pre-protein signal peptide comprising an amino acid sequence of SEQ ID NO: 4 increases the secretion of a payload protein appended thereto as compared to native

### Exemplary Signal Peptides

In some embodiments, a pre-protein signal peptide is provided, wherein the pre-protein signal peptide comprises an amino acid sequence having at least 95% identity to SEQ ID NO: 1. In some embodiments, the pre-protein signal peptide comprising an amino acid sequence having at least 95% identity to SEQ ID NO: 1 increases the secretion of a payload protein appended thereto as compared to native signal peptides. In some embodiments, the pre-protein signal peptide comprising an amino acid sequence having at least 95% identity to SEQ ID NO: 1 comprises an amino acid sequence of SEQ ID NOs: 73-84, as depicted in Table 7 below. In some embodiments, a pre-protein signal peptide comprising an amino acid sequence of SEQ ID NOs: 73-84 increases the secretion of a payload protein appended thereto as compared to native signal peptides.

TABLE 7
Exemplary pre-protein signal peptide sequences
having 95% identity to SEQ ID NO: 1

| SEQ ID NO: | Sequence |
|---|---|
| SEQ ID NO: 73 | MFKLLALGLLALGLLLSSSAQAAD |
| SEQ ID NO: 74 | MKKLLALGLLALGLLLSSSAQAAD |
| SEQ ID NO: 75 | MKKLLALGLLALGLLLSSSVQAAD |
| SEQ ID NO: 76 | MKKLLALGLLAVGLLLSSSAQAAD |
| SEQ ID NO: 77 | MKKLLSLGLLALGLLLSSSAQAAE |
| SEQ ID NO: 78 | MKKLLALGLIALGLLLSSSAQAAD |
| SEQ ID NO: 79 | MKKLLLLGLLALGLLLSSSAQAAE |
| SEQ ID NO: 80 | MKKLLPLGLLALGLLLSSSAQAAD |
| SEQ ID NO: 81 | MKKLLALGLLALGLLFSSSAQAAD |
| SEQ ID NO: 82 | MKKLLALGLCALGLLLSSSAHAAD |
| SEQ ID NO: 83 | MKKLLGLGMLALGLLLSSSAQAAE |
| SEQ ID NO: 84 | MFRLLLALGLLALGLLLSSAQAAD |

In some embodiments, a pre-protein signal peptide is provided, wherein the pre-protein signal peptide comprises an amino acid sequence having at least 95% identity to SEQ ID NO: 2. In some embodiments, the pre-protein signal peptide comprising an amino acid sequence having at least 95% identity to SEQ ID NO: 2 increases the secretion of a payload protein appended thereto as compared to native signal peptides. In some embodiments, the pre-protein signal peptide comprising an amino acid sequence having at least 95% identity to SEQ ID NO: 2 comprises an amino acid sequence of SEQ ID NO: 85 or SEQ ID NO: 86, as depicted in Table 8 below. In some embodiments, a pre-protein signal peptide comprising an amino acid sequence of SEQ ID NO: 85 or SEQ ID NO: 86 increases the secretion of a payload protein appended thereto as compared to native signal peptides.

TABLE 8
Exemplary pre-protein signal peptide sequences
having 95% identity to SEQ ID NO: 2

| SEQ ID NO: | Sequence |
|---|---|
| SEQ ID NO: 85 | MKKIALLTLTLALLLSASSAFAAD |
| SEQ ID NO: 86 | MFKILASLVLAALLLASASAFAAD |

In some embodiments, a pre-protein signal peptide is provided, wherein the pre-protein signal peptide comprises an amino acid sequence having at least 95% identity to SEQ ID NO: 3. In some embodiments, the pre-protein signal peptide comprising an amino acid sequence having at least 95% identity to SEQ ID NO: 3 increases the secretion of a payload protein appended thereto as compared to native signal peptides. In some embodiments, the pre-protein signal peptide comprising an amino acid sequence having at least 95% identity to SEQ ID NO: 3 comprises an amino acid sequence of SEQ ID NOs: 87-138, as depicted in Table 9 below. In some embodiments, a pre-protein signal peptide comprising an amino acid sequence of SEQ ID NOs: 87-138 increases the secretion of a payload protein appended thereto as compared to native signal peptides.

TABLE 9
Exemplary pre-protein signal peptide sequences
having 95% identity to SEQ ID NO: 3

| SEQ ID NO: | Sequence |
|---|---|
| SEQ ID NO: 87 | MKKNLLLASLLLLLALASGSAQAAD |
| SEQ ID NO: 88 | MFKNLLLASLLLLLALASGSAFAAT |
| SEQ ID NO: 89 | MFKNLLLASLLLLLALASGSAQAAD |
| SEQ ID NO: 90 | MKKNLLLASLLLLLALASYSALGED |
| SEQ ID NO: 91 | MKKNSLLASLLLLLALASGSAHAAD |
| SEQ ID NO: 92 | MKKNLLLFSLLLLLALASGSAQAAD |
| SEQ ID NO: 93 | MKRNLLLASLLLLLALASGSAQAAE |
| SEQ ID NO: 94 | MKKNLLLASLMGLLALASGSAQAAD |
| SEQ ID NO: 95 | MKKNLLLASLLLLLTLASGSAQAAD |
| SEQ ID NO: 96 | MKKNLLLASLMLLLALTSGSAQAAE |
| SEQ ID NO: 97 | MKKNLLRASLLLLLALASGSAQAAD |
| SEQ ID NO: 98 | MKRNLLLASLLLLLALASGSAQAAD |
| SEQ ID NO: 99 | MKKNLLLLSLLLLLALASGSAQAAE |
| SEQ ID NO: 100 | MKKNLLLASLLLLLALASGSAAAAD |
| SEQ ID NO: 101 | MKKNLLLASLLLLLALAPGSAQAAD |
| SEQ ID NO: 102 | MKKNLLLASLLLLLALASHSAQAAD |
| SEQ ID NO: 103 | MTKNLLLASLLLLLALASGSAQAAD |
| SEQ ID NO: 104 | MKKNLLLASLVLLLALASGSAHAAD |
| SEQ ID NO: 105 | MKANLLLASLLLLLALASGSAQAAD |
| SEQ ID NO: 106 | MKKNLLLASLALLLALASGSAQAAD |
| SEQ ID NO: 107 | MKKNLLLASLLLAMALASGSAQAAD |
| SEQ ID NO: 108 | MKKNLLLASLVLLLALASGSAQAAD |
| SEQ ID NO: 109 | MKKNLLLASLLLLLALASPSAQAAE |
| SEQ ID NO: 110 | MKKNLLLASLMLLLALASGMAHAAE |
| SEQ ID NO: 111 | MKKNILLASLLLLLALASGSAQAAD |
| SEQ ID NO: 112 | MKKNLLLASLLLLLALASMSAQAAD |
| SEQ ID NO: 113 | MKKNLLLASLLLLLAVASGSAQAAE |
| SEQ ID NO: 114 | MKKNLLLLSLLLLLALASGSAQAAD |
| SEQ ID NO: 115 | MKKFLLLASLLLLLALASGSAQAAE |
| SEQ ID NO: 116 | MKKNLLLAVLLLLLALASGSAQAAD |
| SEQ ID NO: 117 | MKKNLLFASLLLLLALASGSAQAAD |
| SEQ ID NO: 118 | MKKNLLLASLLLCLALASGSAQAAD |
| SEQ ID NO: 119 | MKKNLLLASLLLLLAFASGSAQAAD |
| SEQ ID NO: 120 | MKKNLLAASLLLLLALASGSAQAAD |
| SEQ ID NO: 121 | MKKNLALASLLLLLALASGSAQAAE |
| SEQ ID NO: 122 | MKKNLLLASILLLLALASGSAHAAD |
| SEQ ID NO: 123 | MKKNLLLASLLPLLALASGSAQAAD |
| SEQ ID NO: 124 | MKKNILLASLLLLLAMASGSAQAAD |
| SEQ ID NO: 125 | MKKNSLLASLLLLLALASGSAQAAE |
| SEQ ID NO: 126 | MKKNLLLASLLLVLALASGSAQAAD |
| SEQ ID NO: 127 | MKKNLLLASLLMALALASGSAQAAD |
| SEQ ID NO: 128 | MKKNLLLASLLLLLALASGSAQADD |
| SEQ ID NO: 129 | MKKNLLLASLLILLALASGSAQAAD |
| SEQ ID NO: 130 | MKKNLLLASLLLLCALASGSAQAAD |
| SEQ ID NO: 131 | MKKNLLLASLVLLLALASGSAQAAE |
| SEQ ID NO: 132 | MKKNLLLASLLLALALASGSAQAAD |
| SEQ ID NO: 133 | MKKNLLLASLLLLLALASGSGQAAD |
| SEQ ID NO: 134 | MKKNLLLASLLLLLALAAGSAQAAK |
| SEQ ID NO: 135 | MFKTLLLASLLLLLALASGSAQAAD |
| SEQ ID NO: 136 | MFKNLLLASLLLLLALASGSAQADD |
| SEQ ID NO: 137 | MFRNLLLLSLLLLLALASGSAQAAD |
| SEQ ID NO: 138 | MKKNLLLAVLLLLLALASGSVFAED |

In some embodiments, a pre-protein signal peptide is provided, wherein the pre-protein signal peptide comprises an amino acid sequence having at least 95% identity to SEQ ID NO: 4. In some embodiments, the pre-protein signal peptide comprising an amino acid sequence having at least 95% identity to SEQ ID NO: 4 increases the secretion of a payload protein appended thereto as compared to native signal peptides. In some embodiments, the pre-protein signal peptide comprising an amino acid sequence having at least 95% identity to SEQ ID NO: 4 comprises an amino acid sequence of SEQ ID NOs: 139-217, as depicted in Table 10 below. In some embodiments, a pre-protein signal peptide comprising an amino acid sequence of SEQ ID NOs: 139-217 increases the secretion of a payload protein appended thereto as compared to native signal peptides.

TABLE 10
Exemplary pre-protein signal peptide sequences
having 95% identity to SEQ ID NO: 4

| SEQ ID NO: | Sequence |
|---|---|
| SEQ ID NO: 139 | MKKIKLLLLLLLLALLLSGSAFAAD |
| SEQ ID NO: 140 | MKKIKLLLLLLLLALLLSGSAFAAG |
| SEQ ID NO: 141 | MFKIKLLLLLLLLALLLSGSAFAAD |
| SEQ ID NO: 142 | MFKIKLLLLLLLLALLLSGSAFAAE |
| SEQ ID NO: 143 | MKKIKLLLLLLLLALLLSGSAFAAT |
| SEQ ID NO: 144 | MFKIRPLCLLLLLALLLSGSAFAAD |
| SEQ ID NO: 145 | MFRLKLLLLLLMLALLLSGSAFAAD |
| SEQ ID NO: 146 | MKKIKLLLLLLLLALLLSGSAQAKD |
| SEQ ID NO: 147 | MKKIKLLLLLLLAALLLSGSAFAAD |
| SEQ ID NO: 148 | MKKIKLLLLLLLLAGLLSGSAFAAD |
| SEQ ID NO: 149 | MKKIKLLLLLLLLALLLSLSAYAAD |
| SEQ ID NO: 150 | MKKIKLLLLLLLLALLLSGSVFAAD |
| SEQ ID NO: 151 | MKKIKLLLLLLLLALLLSGVAFAAD |
| SEQ ID NO: 152 | MTKIKLLLLLLLLALLLSGSAFAAD |
| SEQ ID NO: 153 | MKKIKLLLLLLLLALLLSLSAFAAD |
| SEQ ID NO: 154 | MKKIKLLLLLLLLALQLSGSAFAAD |
| SEQ ID NO: 155 | MKKIRLLLLLLLLALLLSGSAFAAD |
| SEQ ID NO: 156 | MKRIKLLMLLLLLALLLSGSAFAAD |
| SEQ ID NO: 157 | MKKIKLLFLLLLLALLLSGSAFAAD |
| SEQ ID NO: 158 | MKKIKLLLLLALLALLLSGSAFAAD |
| SEQ ID NO: 159 | MKKIYLLLLLLLLALLLSGSAFAAD |
| SEQ ID NO: 160 | MKKIKLLLMLLLLALLLAGSAFAAD |
| SEQ ID NO: 161 | MKKIKLLLLALLLALLLSGSAFAAD |
| SEQ ID NO: 162 | MKKIKLLLLLLLLALPLSGSAFAAD |
| SEQ ID NO: 163 | MKKLKLLLLLLLLALLLSGSAFAAD |
| SEQ ID NO: 164 | MKKIKLLGLLLLLAMLLSGSAFAAE |
| SEQ ID NO: 165 | MTKIKLLLLLLLLALLLSGSAFAAE |
| SEQ ID NO: 166 | MYKIKLLLLLLLLAMLLSGSAFAAE |
| SEQ ID NO: 167 | MKKLKLLLLLLLLALLLSGSAFAAE |
| SEQ ID NO: 168 | MKKIKLLLLLLLLALCLSGSAFAAD |
| SEQ ID NO: 169 | MKKIKLLLLLLLLSLLLSGSAFAAD |
| SEQ ID NO: 170 | MKKVKLLLLLLLLALLWSGSAFAAD |
| SEQ ID NO: 171 | MKRIKLLLLLLLLALLLSGSAFAAD |
| SEQ ID NO: 172 | MKKIKLLLLLLLVALLLSGSAFAAE |
| SEQ ID NO: 173 | MKKHKLLLLLLLLALLLSGSAFAAD |
| SEQ ID NO: 174 | MKKIKLLLLLLLLALLLAGSAFAAD |
| SEQ ID NO: 175 | MKKIKLLLLLLLLLLLLSGSAFAAD |
| SEQ ID NO: 176 | MKKIKLLGLLLLLALLLSGSAFAAE |
| SEQ ID NO: 177 | MKKIKLLLLLLLLALLVSGSAFAAD |
| SEQ ID NO: 178 | MKKIKLLLLLLLLALLASGSAFAAD |
| SEQ ID NO: 179 | MKKIKLLLLLLLLALLLSGSAFADD |
| SEQ ID NO: 180 | MKKIKLLLSLLLLALLLSGSAFAAD |
| SEQ ID NO: 181 | MNKIKLLLLLLLLALLLSGSAFAAD |
| SEQ ID NO: 182 | MKKIKLLLLLLLYALLLSGSAFAAD |
| SEQ ID NO: 183 | MKKIKLLLLLLLLALLLSGSTFAAD |
| SEQ ID NO: 184 | MKKIKLLLLLLLLALLLPGSAFAAD |
| SEQ ID NO: 185 | MKKIKLLLALLLLALLLSGSAFAAD |
| SEQ ID NO: 186 | MKKFKLLLLLLLLALLLSGSAFAAD |
| SEQ ID NO: 187 | MKKIKLLLLLLLLALLLSGSLFAAD |
| SEQ ID NO: 188 | MKKIKLLLLLLLLALLLSGSAFAQD |
| SEQ ID NO: 189 | MYKIKLLLLLLLLALLLSGSAFAAD |
| SEQ ID NO: 190 | MKKIKLLLLLLILALLLSGSAFAAD |
| SEQ ID NO: 191 | MKKVKLLLLALLLALLLSGSAFAAD |
| SEQ ID NO: 192 | MKKIKLILLLLLLALLLSGSAFAAD |
| SEQ ID NO: 193 | MKKIKLLLLALMLALLLSGSAFAAE |
| SEQ ID NO: 194 | MKKIKLLLLLLVLALLLSGSAFAAD |
| SEQ ID NO: 195 | MKKIKLLCLLLLLALLLSGSAFAAD |
| SEQ ID NO: 196 | MKKIKLALLLLLLALLLSGSAFAAD |
| SEQ ID NO: 197 | MKKIKLLLLLLLAALLLSGSAFAAG |
| SEQ ID NO: 198 | MKKIKLLLLALLLALLLSGSAFAAI |
| SEQ ID NO: 199 | MKKIKLLLLLLLLALLYSGSAFAAK |
| SEQ ID NO: 200 | MFKIKLLLLLLLLALLLSGSAFGAD |
| SEQ ID NO: 201 | MFKIKLLLLLLLLFLLLSGSAFAAD |
| SEQ ID NO: 202 | MFKIKLLLLLLLLSLLLSGSAFAAD |
| SEQ ID NO: 203 | MFKLKLLLLLLLLALLLSGSAFAAD |
| SEQ ID NO: 204 | MFKIKLLLLLLLLALLLPGSAFAAD |
| SEQ ID NO: 205 | MFKIKMLLLLLLLGLLLSGSAFAAD |
| SEQ ID NO: 206 | MFKIKLLLLLLLLALLLCGSAFAAD |
| SEQ ID NO: 207 | MFKIKLLLLLLLLALLLAGSAFAAE |
| SEQ ID NO: 208 | MFKIKLLLLALLLALLLSGSAFAAD |
| SEQ ID NO: 209 | MFKIKLLLLLLLLAALLSGSAFAAD |
| SEQ ID NO: 210 | MFKIKLLLLLLLLALLLSLSAFAAD |
| SEQ ID NO: 211 | MFKYKLLLLLLLLALLLSGSAFAAE |
| SEQ ID NO: 212 | MFKIKLLLLLLLLALLLPGSAFAAE |
| SEQ ID NO: 213 | MFKIKLLLLLLALALLLSGSAFAAD |
| SEQ ID NO: 214 | MFKIKLLLLALLLALLLSGSAFAAM |
| SEQ ID NO: 215 | MYLIKLLLLLLLLALLLAGSAFAAD |
| SEQ ID NO: 216 | MFKIKLLLLCLLLALLLSGSAFAEE |
| SEQ ID NO: 217 | MFRIKLLLLLLLLSLLLSGSAFAGE |

### Polypeptides

In some embodiments, a polypeptide is provided. In some embodiments, the polypeptide comprises a formula of X₁—Z₁, wherein X₁ is a pre-protein signal peptide and Z₁ is a payload protein. In some embodiments, the polypeptide is a recombinant polypeptide. In some embodiments, the recombinant polypeptide comprises a formula of X₁—Z₁, wherein X₁ is a pre-protein signal peptide and Z₁ is a payload protein.

In some embodiments, the polypeptide comprises a formula of X₁—Z₁, wherein X₁ comprises an amino acid sequence of Formula I. In some embodiments, the polypeptide comprises a formula of X₁—Z₁, wherein X₁ comprises an amino acid sequence having at last 70% identity to an amino acid sequence selected from the group consisting of SEQ ID NO: 1, 2, 3, or 4. In some embodiments, the polypeptide comprises a formula of X₁—Z₁, wherein X₁ comprises an amino acid sequence having at least at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to an amino acid sequence selected from the group consisting of SEQ ID NO: 1, 2, 3, or 4. In some embodiments, the polypeptide comprises a formula of X₁—Z₁, wherein X₁ comprises an amino acid sequence selected from the group consisting of SEQ ID NO: 1, 2, 3 and 4. In some embodiments, the polypeptide comprises a formula of X₁—Z₁, wherein X₁ comprises an amino acid sequence of SEQ ID NO: 1. In some embodiments, the polypeptide comprises a formula of X₁—Z₁, wherein X₁ comprises an amino acid sequence of SEQ ID NO: 2. In some embodiments, the polypeptide comprises a formula of X₁—Z₁, wherein X₁ comprises an amino acid sequence of SEQ ID NO: 3. In some embodiments, the polypeptide comprises a formula of X₁—Z₁, wherein X₁ comprises an amino acid sequence of SEQ ID NO: 4.

In some embodiments, the polypeptide comprises a formula of X₁—Z₁, wherein Z₁ is any peptide or protein. In some embodiments, the polypeptide comprises a formula of X₁—Z₁, wherein Z₁ is selected from the group including, but not limited to, an antiviral, insulin, an incretin, an enzyme, an enzyme inhibitor, a hormone, a cytokine, an antibody, a single domain antibody fragment, a single chain variable antibody fragment, an antimicrobial peptide, a mucosal protein, pesticide, bactericide herbicide, fungicide, nematicide, miticide, plant growth regulator, plant growth stimulator or fertilizer, a vaccine, a diagnostic protein, a feed conversion enzyme, a flavoring, a nutritional protein, venom peptides, endoglucanase, restriction enzymes, human growth hormone, insulin, Beta-lactamase, luciferase (such as, but not limited to, Renilla luciferase or NanoLuc luciferase), phytase, cellulase, chitinase, phosphatase, catalase, urokinase, tissue plasminogen activator, apolipoprotein, beta-glucosidases, hemicellulases, lignocellulose oxireductases, DNAses NADPH dehydrogenase, alcohol oxidase, pyruvate decarboxylase, formolase, alpha-ketoglutarate dehydrogenase, branched chain alpha-ketoacid decarboxylase, copper radical oxidase, galactose oxidase, glycerol oxidase, amine oxidase, glyoxalase, amino monoaxidase, ethylene glycol oxidase, alditol oxidase, or 2-oxoglutarate dehydrogenase. In some embodiments, the polypeptide comprises a formula of X₁—Z₁, wherein Z₁ is selected from the group including, but not limited to, an enzyme (e.g., invertase, isomaltase, lactase, lysozyme, An-PEP), a growth factor (e.g., IGF1), insulin, an incretin (e.g., GLP-1, GLP-2, leptin, apelin, ghrelin, PYY, nesfatin), a cytokine, an antibody, an antimicrobial peptide), a mucosal protein (e.g., trefoil factor, Reg3 protein, superoxide dismutase), an agricultural product (e.g., pesticide, bactericide herbicide, fungicide, nematicide, miticide, plant growth regulator, plant growth stimulator, or fertilizer), a vaccine, a diagnostic protein, a feed conversion enzyme, a flavoring, or a nutritional protein. In some embodiments, the polypeptide comprises a formula of X₁—Z₁, wherein Z₁ is selected from the group including, but not limited to, venom peptides, endoglucanase, restriction enzymes, human growth hormone, insulin, Beta-lactamase, luciferase (such as, but not limited to, Renilla luciferase or NanoLuc luciferase), phytase, cellulase, chitinase, phosphatase, catalase, urokinase, tissue plasminogen activator, apolipoprotein, NADPH dehydrogenase, alcohol oxidase, pyruvate decarboxylase, formolase, alpha-ketoglutarate dehydrogenase, branched chain alpha-ketoacid decarboxylase, copper radical oxidase, galactose oxidase, glycerol oxidase, amine oxidase, glyoxalase, amino monoaxidase, ethylene glycol oxidase, alditol oxidase, or 2-oxoglutarate dehydrogenase. In some embodiments, the polypeptide comprises a formula of X₁—Z₁, wherein Z₁ is selected from the group including, but not limited to, amylases, alpha amylases, xylanases (e.g. endo-1,4-beta-xylanase), lichenases (e.g. beta glucanase), lipases (e.g. Candida antartica lipase B, Candida rugose lipase, LipA), pectinases (e.g. pectate trisaccharide lyase), and cellulases (e.g. endoglucanase A). The examples listed are provided for clarity only and are not meant to be limiting in any way. Thus, for example, the current disclosure is not limited to IGF-1 for “growth factor”, but rather encompasses and includes all growth factors known in the art.

In some embodiments, the polypeptide comprises a formula of X₁—Z₁, wherein Z₁ comprises an amino acid sequence having at least 70% identity to SEQ ID NO: 28:

(SEQ ID NO: 28)
APVNTTTEDETAQIPAEAVIGYSDLEGDEDVAVLPESNSTNNGLLFINTTIASIAAKEEGVS
LDKREEGEPKSMTNETSDRPLVHFTPNKGWMNDPNGLWYDEKDAKWHLYFQYNPNDTVWGTP
LFWGHATSDDLTNWEDQPIAIAPKRNDSGAFSGSMVVDYNNTSGFENDTIDPRQRCVAIWTY
NTPESEEQYISYSLDGGYTFTEYQKNPVLAANSTQFRDPKVEWYEPSQKWIMTAAKSQDYKI
EIYSSDDLKSWKLESAFANEGFLGYQYECPGLIEVPTEQDPSKSYWVMFISINPGAPAGGSF
NQYFVGSENGTHFEAFDNQSRVVDEGKDYYALQTFENTDPTYGSALGIAWASNWEYSAFVPT
NPWRSSMSLVRKESLNTEYQANPETELINLKAEPILNISNAGPWSRFATNTTLTKANSYNVD
LSNSTGTLEFELVYAVNTTQTISKSVFADLSLWEKGLEDPEEYLRMGFEVSASSFELDRGNS
KVKFVKENPYFTNRMSVNNQPFKSENDLSYYKVYGLLDQNILELYENDGDVVSTNTYFMTTG
NALGSVNMTTGVDNLFYIDKFQVREVK

or is substantially similar to SEQ ID NO: 28 or is an active fragment of SEQ ID NO: 28. In some embodiments, the polypeptide comprises a formula of X₁—Z₁, wherein Z₁ comprises an amino acid sequence having least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 28. In some embodiments, the polypeptide comprises a formula of X₁—Z₁, wherein Z₁ comprises an amino acid sequence of SEQ ID NO: 28.

In some embodiments, the polypeptide comprises a formula of X₁—Z₁, wherein Z₁ comprises an amino acid sequence having at least 70% identity to SEQ ID NO: 29:

(SEQ ID NO: 29)
SMTNETSDRPLVHFTPNKGWMNDPNGLWYDEKDAKWHLYFQYNPNDTVWGTPLFWGHATSDD
LTNWEDQPIAIAPKRNDSGAFSGSMVVDYNNTSGFENDTIDPRQRCVAIWTYNTPESEEQYI
SYSLDGGYTFTEYQKNPVLAANSTQFRDPKVEWYEPSQKWIMTAAKSQDYKIEIYSSDDLKS
WKLESAFANEGFLGYQYECPGLIEVPTEQDPSKSYWVMFISINPGAPAGGSFNQYFVGSENG
THFEAFDNQSRVVDEGKDYYALQTFFNTDPTYGSALGIAWASNWEYSAFVPTNPWRSSMSLV
RKFSLNTEYQANPETELINLKAEPILNISNAGPWSRFATNTTLTKANSYNVDLSNSTGTLEF
ELVYAVNTTQTISKSVFADLSLWFKGLEDPEEYLRMGFEVSASSFFLDRGNSKVKFVKENPY
FTNRMSVNNQPFKSENDLSYYKVYGLLDQNILELYFNDGDVVSTNTYFMTTGNALGSVNMTT
GVDNLFYIDKFQVREVK

or is substantially similar to SEQ ID NO: 29 or is an active fragment of SEQ ID NO: 29. In some embodiments, the polypeptide comprises a formula of X₁—Z₁, wherein Z₁ comprises an amino acid sequence having least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 29. In some embodiments, the polypeptide comprises a formula of X₁—Z₁, wherein Z₁ comprises an amino acid sequence of SEQ ID NO: 29.

In some embodiments, the polypeptide comprises a formula of X₁—Z₁, wherein Z₁ comprises an amino acid sequence having at least 70% identity to SEQ ID NO: 30:

(SEQ ID NO: 30)
KVFERCELARTLKRLGMDGYRGISLANWMCLAKWESGYNTRATNYNAGDR
STDYGIFQINSRYWCNDGKTPGAVNACQLSCSALLQDNIADAVACAKRVV
RDPQGIRAWVAWRNRCONRDVRQYVQGCGV

or is substantially similar to SEQ ID NO: 30 or is an active fragment of SEQ ID NO: 30. In some embodiments, the polypeptide comprises a formula of X₁—Z₁, wherein Z₁ comprises an amino acid sequence having least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 30. In some embodiments, the polypeptide comprises a formula of X₁—Z₁, wherein Z₁ comprises an amino acid sequence of SEQ ID NO: 30.

In some embodiments, the polypeptide comprises a formula of X₁—Z₁, wherein Z₁ comprises an amino acid sequence having at least 70% identity to SEQ ID NO: 31:

(SEQ ID NO: 31)
IKHRLNGFTILEHPDPAKRDLLQDIVTWDDKSLFINGERIMLESGEVHPFRLPVPSLWLDIF
HKIRALGFNCVSFYIDWALLEGKPGDYRAEGIFALEPFFDAAKEAGIYLIARPGSYINAEVS
GGGFPGWLQRVNGTLRSSDEPFLKATDNYIANAAAAVAKAQITNGGPVILYQPENEYSGGCC
GVKYPDADYMQYVMDQARKADIVVPFISNDASPSGHNAPGSGTSAVDIYGHDSYPLGFDCAN
PSVWPEGKLPDNERTLHLEQSPSTPYSLLEFQAGAFDPWGGPGFEKCYALVNHEFSRVFYRN
DLSFGVSTFNLYMTFGGTNWGNLGHPGGYTSYDYGSPITETRNVTREKYSDIKLLANFVKAS
PSYLTATPRNLTTGVYTDTSDLAVTPLIGDSPGSFFVVRHTDYSSQESTSYKLKLPTSAGNL
TIPQLEGTLSLNGRDSKIHVVDYNVSGTNIIYSTAEVFTWKKEDGNKVLVLYGGPKEHHELA
IASKSNVTIIEGSDSGIVSTRKGSSVIIGWDVSSTRRIVQVGDLRVELLDRNSAYNYWVPEL
PTEGTSPGFSTSKTTASSIIVKAGYLLRGAHLDGADLHLTADENATTPIEVIGAPTGAKNLF
VNGEKASHTVDKNGIWSSEVKYAAPEIKLPGLKDLDWKYLDTLPEIKSSYDDSAWVSADLPK
TKNTHRPLDTPTSLYSSDYGFHTGYLIYRGHFVANGKESEFFIRTQGGSAFGSSVWLNETYL
GSWTGADYAMDGNSTYKLSQLESGKNYVITVVIDNLGLDENWTVGEETMKNPRGILSYKLSG
QDASAITWKLTGNLGGEDYQDKVRGPLNEGGLYAERQGFHQPQPPSESWESGSPLEGLSKPG
IGFYTAQFDLDLPKGWDVPLYFNFGNNTQAARAQLYVNGYQYGKFTGNVGPQTSFPVPEGIL
NYRGTNYVALSLWALESDGAKLGSFELSYTTPVLTGYGNVESPEQPKYEQRKGAY

or is substantially similar to SEQ ID NO: 31 or is an active fragment of SEQ ID NO: 31. In some embodiments, the polypeptide comprises a formula of X₁—Z₁, wherein Z₁ comprises an amino acid sequence having least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 31. In some embodiments, the polypeptide comprises a formula of X₁—Z₁, wherein Z₁ comprises an amino acid sequence of SEQ ID NO: 31.

In some embodiments, the polypeptide comprises a formula of X₁—Z₁, wherein Z₁ comprises an amino acid sequence having at least 70% identity to SEQ ID NO: 32:

(SEQ ID NO: 32)
EVQLVESGGGLVQPGGSLRLSCAASGFTFSDYWMYWVRQAPGKGLEWVSE
INTNGLITKYPDSVGRFTISRDNAKNTLYLQMNSLRPEDTAVYYCARSPS
GFNRGQGTLVTVSS

or is substantially similar to SEQ ID NO: 32 or is an active fragment of SEQ ID NO: 32. In some embodiments, the polypeptide comprises a formula of X₁—Z₁, wherein Z₁ comprises an amino acid sequence having least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 32. In some embodiments, the polypeptide comprises a formula of X₁—Z₁, wherein Z₁ comprises an amino acid sequence of SEQ ID NO: 32.

In some embodiments, the polypeptide comprises a formula of X₁—Z₁, wherein Z₁ comprises an amino acid sequence having at least 70% identity to SEQ ID NO: 33:

(SEQ ID NO: 33)
IEEGKLVIWINGDKGYNGLAEVGKKFEKDTGIKVTVEHPDKLEEKFPQVA
ATGDGPDIIFWAHDRFGGYAQSGLLAEITPDKAFQDKLYPFTWDAVRYNG
KLIAYPIAVEALSLIYNKDLLPNPPKTWEEIPALDKELKAKGKSALMFNL
QEPYFTWPLIAADGGYAFKYENGKYDIKDVGVDNAGAKAGLTFLVDLIKN
KHMNADTDYSIAEAAFNKGETAMTINGPWAWSNIDTSKVNYGVTVLPTFK
GQPSKPFVGVLSAGINAASPNKELAKEFLENYLLTDEGLEAVNKDKPLGA
VALKSYEEELAKDPRIAATMENAQKGEIMPNIPQMSAFWYAVRTAVINAA
SGRQTVDEALKDAQTRITK

or is substantially similar to SEQ ID NO: 33 or is an active fragment of SEQ ID NO: 33. In some embodiments, the polypeptide comprises a formula of X₁—Z₁, wherein Z₁ comprises an amino acid sequence having least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 33. In some embodiments, the polypeptide comprises a formula of X₁—Z₁, wherein Z₁ comprises an amino acid sequence of SEQ ID NO: 33.

In some embodiments, the polypeptide comprises a formula of X₁—Z₁, wherein Z₁ comprises an amino acid sequence having at least 70% identity to SEQ ID NO: 34:

(SEQ ID NO: 34)
AQSEPELKLESVVIVSRHGVRAPTKATQLMQDVTPDAWPTWPVKLGELTP
RGGELLAYLGHYWRQRLVADGLLPKCGCPQSGQVAILADVDERTRKTGEA
FAAGLAPDCAITVHTQADTSSPDPLFNPLKTGVCQLDNANVIDAILERAG
GSLADFTGHYQTAFRELERVLNFPQSNLCLKREKQDESCSLTQALPSELK
VSADCVSLTGAVSLASMLTEIFLLQQAQGMPEPGWGRITDSHQWNTLLSL
HNAQFDLLQRTPEVARSRATPLLDLIKTALTPHPPQKQAYGVTLPTSVLF
LAGHDTNLANLGGALELNWTLPGQPDNTPPGGELVFERWRRLSDNSQWIQ
VSLVFQTLQQMRDKTPLSLNTPPGEVKLTLAGCEERNAQGMCSLAGFTQI
VNEARIPACSL

or is substantially similar to SEQ ID NO: 34 or is an active fragment of SEQ ID NO: 34. In some embodiments, the polypeptide comprises a formula of X₁—Z₁, wherein Z₁ comprises an amino acid sequence having least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 34. In some embodiments, the polypeptide comprises a formula of X₁—Z₁, wherein Z₁ comprises an amino acid sequence of SEQ ID NO: 34.

In some embodiments, the polypeptide comprises a formula of X₁—Z₁, wherein Z₁ comprises an amino acid sequence having at least 70% identity to SEQ ID NO: 35:

(SEQ ID NO: 35)
FVNQHLCGSHLVEALYLVCGERGFFYTPKEWKGIVEQCCTSICSLYQLEN
YCN

or is substantially similar to SEQ ID NO: 35 or is an active fragment of SEQ ID NO: 35. In some embodiments, the polypeptide comprises a formula of X₁—Z₁, wherein Z₁ comprises an amino acid sequence having least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 35. In some embodiments, the polypeptide comprises a formula of X₁—Z₁, wherein Z₁ comprises an amino acid sequence of SEQ ID NO: 35.

In some embodiments, the polypeptide comprises a formula of X₁—Z₁, wherein Z₁ comprises an amino acid sequence having at least 70% identity to SEQ ID NO: 36:

(SEQ ID NO: 36)
GPETLCGAELVDALQFVCGPRGFYENKPTGYGSSIRRAPQTGIVDECCFR
SCDLRRLEMYCAPLKPTKAARSIRAQRHTDMPKTQKEVHLKNTSRGSAGN
KTYRM

or is substantially similar to SEQ ID NO: 36 or is an active fragment of SEQ ID NO: 36. In some embodiments, the polypeptide comprises a formula of X₁—Z₁, wherein Z₁ comprises an amino acid sequence having least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 36. In some embodiments, the polypeptide comprises a formula of X₁—Z₁, wherein Z₁ comprises an amino acid sequence of SEQ ID NO: 36.

In some embodiments, the polypeptide comprises a formula of X₁—Z₁, wherein Z₁ comprises an amino acid sequence having at least 70% identity to SEQ ID NO: 37:

(SEQ ID NO: 37)
KVFERCELARTLKRLGMDGYRGISLANWMCLAKWESGYNTRATNYNAGDR
STDYGIFQINSRYWCNDGKTPGAVNACQLSCSALLQDNIADAVACAKRVV
RDPQGIRAWVAWRNRCQNRDVRQYVQGCGV

or is substantially similar to SEQ ID NO: 37 or is an active fragment of SEQ ID NO: 37. In some embodiments, the polypeptide comprises a formula of X₁—Z₁, wherein Z₁ comprises an amino acid sequence having least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 37. In some embodiments, the polypeptide comprises a formula of X₁—Z₁, wherein Z₁ comprises an amino acid sequence of SEQ ID NO: 37.

In some embodiments, the polypeptide comprises a formula of X₁—Z₁, wherein Z₁ comprises an amino acid sequence having at least 70% identity to SEQ ID NO: 38:

(SEQ ID NO: 38)
MKRSISIFITCLLITLLIMGGMIASPASAAGTKTPVAKNGQLSIKGTQLV
NRDGKAVQLKGISSHGLQWYGEYVNKDSLKWLRDDWGITVFRAAMYTADG
GYIDNPSVKNKVKEAVEAAKELGIYVIIDWHILNDGNPNQNKEKAKEFFK
EMSSLYGNTPNVIYEIANEPNGDVNWKRDIKPYAEEVISVIRKNDPDNII
IVGTGTWSQDVNDAADDQLKDANVMYALHFYAGTHGQFLRDKANYALSKG
APIFVTEWGTSDASGNGGVFLDQSREWLKYLDSKTISWVNWNLSDKQESS
SALKPGASKTGGWRLSDLSASGTFVRENILGTKDSTKDIPETPSKDKPTQ
ENGISVQYRAGDGSMNSNQIRPQLQIKNNGNTTVDLKDVTARYWYKAKNK
GQNFDCDYAQIGCGNVTHKFVTLHKPKQGADTYLELGFKNGTLAPGASTG
NIQLRLHNDDWSNYAQSGDYSFFKSNTFKTTKKITLYDQGKLIWGTEPN

or is substantially similar to SEQ ID NO: 38 or is an active fragment of SEQ ID NO: 38. In some embodiments, the polypeptide comprises a formula of X₁—Z₁, wherein Z₁ comprises an amino acid sequence having least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 38. In some embodiments, the polypeptide comprises a formula of X₁—Z₁, wherein Z₁ comprises an amino acid sequence of SEQ ID NO: 38.

In some embodiments, the polypeptide comprises a formula of X₁—Z₁, wherein Z₁ comprises an amino acid sequence having at least 70% identity to SEQ ID NO: 39:

(SEQ ID NO: 39)
MKQQKRLYARLLTLLFALIFLLPHSAAAAANLNGTLMQYFEWYMPNDGQH
WKRLQNDSAYLAEHGITAVWIPPAYKGTSQADVGYGAYDLYDLGEFHQKG
TVRTKYGTKGELQSAIKSLHSRDINVYGDVVINHKGGADATEDVTAVEVD
PADRNRVISGEHRIKAWTHFHFPGRGSTYSDFKWHWYHFDGTDWDESRKL
NRIYKFQGKAWDWEVSNENGNYDYLMYADIDYDHPDVAAEIKRWGTWYAN
ELQLDGFRLDAVKHIKFSFLRDWVNHVREKTGKEMFTVAEYWQNDLGALE
NYLNKTNFNHSVFDVPLHYQFHAASTQGGGYDMRKLLNSTVVSKHPLKAV
TFVDNHDTQPGQSLESTVQTWFKPLAYAFILTRESGYPQVFYGDMYGTKG
DSQREIPALKHKIEPILKARKQYAYGAQHDYFDHHDIVGWTREGDSSVAN
SGLAALITDGPGGAKRMYVGRQNAGETWHDITGNRSEPVVINSEGWGEFH
VNGGSVSIYVQR

or is substantially similar to SEQ ID NO: 39 or is an active fragment of SEQ ID NO: 39. In some embodiments, the polypeptide comprises a formula of X₁—Z₁, wherein Z₁ comprises an amino acid sequence having least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 39. In some embodiments, the polypeptide comprises a formula of X₁—Z₁, wherein Z₁ comprises an amino acid sequence of SEQ ID NO: 39.

In some embodiments, the polypeptide comprises a formula of X₁—Z₁, wherein Z₁ comprises an amino acid sequence having at least 70% identity to SEQ ID NO: 40:

(SEQ ID NO: 40)
MFKFKKNFLVGLSAALMSISLFSATASAASTDYWQNWTDGGGIVNAVNGS
GGNYSVNWSNTGNFVVGKGWTTGSPFRTINYNAGVWAPNGNGYLTLYGWT
RSPLIEYYVVDSWGTYRPTGTYKGTVKSDGGTYDIYTTTRYNAPSIDGDR
TTFTQYWSVRQSKRPTGSNATITFSNHVNAWKSHGMNLGSNWAYQVMATE
GYQSSGSSNVTVW

or is substantially similar to SEQ ID NO: 40 or is an active fragment of SEQ ID NO: 40. In some embodiments, the polypeptide comprises a formula of X₁—Z₁, wherein Z₁ comprises an amino acid sequence having least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 40. In some embodiments, the polypeptide comprises a formula of X₁—Z₁, wherein Z₁ comprises an amino acid sequence of SEQ ID NO: 40.

In some embodiments, the polypeptide comprises a formula of X₁—Z₁, wherein Z₁ comprises an amino acid sequence having at least 70% identity to SEQ ID NO: 41:

(SEQ ID NO: 41)
MPYLKRVLLLLVTGLEMSLFAVTATASAQTGGSFFDPENGYNSGFWQKAD
GYSNGNMENCTWRANNVSMTSLGEMRLALTSPAYNKFDCGENRSVQTYGY
GLYEVRMKPAKNTGIVSSFFTYTGPTDGTPWDEIDIEFLGKDTTKVQFNY
YTNGAGNHEKIVDLGFDAANAYHTYAFDWQPNSIKWYVDGQLKHTATNQI
PTTPGKIMMNLWNGTGVDEWLGSYNGVNPLYAHYDWVRYTKK

or is substantially similar to SEQ ID NO: 41 or is an active fragment of SEQ ID NO: 41. In some embodiments, the polypeptide comprises a formula of X₁—Z₁, wherein Z₁ comprises an amino acid sequence having least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 41. In some embodiments, the polypeptide comprises a formula of X₁—Z₁, wherein Z₁ comprises an amino acid sequence of SEQ ID NO: 41.

In some embodiments, the polypeptide comprises a formula of X₁—Z₁, wherein Z₁ comprises an amino acid sequence having at least 70% identity to SEQ ID NO: 42:

(SEQ ID NO: 42)
MKFVKRRIIALVTILMLSVTSLFALQPSAKAAEHNPVVMVHGIGGASFNF
AGIKSYLVSQGWSRDKLYAVDFWDKTGTNYNNGPVLSRFVQKVLDETGAK
KVDIVAHSMGGANTLYYIKNLDGGNKVANVVTLGGANRLTTGKALPGTDP
NQKILYTSIYSSADMIVMNYLSRLDGARNVQIHGVGHIGLLYSSQVNSLI
KEGLNGGGQNTN

or is substantially similar to SEQ ID NO: 42 or is an active fragment of SEQ ID NO: 42. In some embodiments, the polypeptide comprises a formula of X₁—Z₁, wherein Z₁ comprises an amino acid sequence having least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 42. In some embodiments, the polypeptide comprises a formula of X₁—Z₁, wherein Z₁ comprises an amino acid sequence of SEQ ID NO: 42.

In some embodiments, the polypeptide comprises a formula of X₁—Z₁, wherein Z₁ comprises an amino acid sequence having at least 70% identity to SEQ ID NO: 43:

(SEQ ID NO: 43)
MKKLISIIFIFVLGVVGSLTAAVSAEAASALNSGKVNPLADFSLKGFAAL
NGGTTGGEGGQTVTVTTGDQLIAALKNKNANTPLKIYVNGTITTSNTSAS
KIDVKDVSNVSIVGSGTKGELKGIGIKIWRANNIIIRNLKIHEVASGDKD
AIGIEGPSKNIWVDHNELYHSLNVDKDYYDGLFDVKRDAEYITFSWNYVH
DGWKSMLMGSSDSDNYNRTITFHHNWFENLNSRVPSFREGEGHIYNNYFN
KIIDSGINSRMGARIRIENNLFENAKDPIVSWYSSSPGYWHVSNNKFVNS
RGSMPTTSTTTYNPPYSYSLDNVDNVKSIVKQNAGVGKINP

or is substantially similar to SEQ ID NO: 43 or is an active fragment of SEQ ID NO: 43. In some embodiments, the polypeptide comprises a formula of X₁—Z₁, wherein Z₁ comprises an amino acid sequence having least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 43. In some embodiments, the polypeptide comprises a formula of X₁—Z₁, wherein Z₁ comprises an amino acid sequence of SEQ ID NO: 43.

In some embodiments, the polypeptide comprises a formula of X₁—Z₁, wherein Z₁ comprises an amino acid sequence having at least 70% identity to SEQ ID NO: 44:

(SEQ ID NO: 44)
MKNVKKRVGVVLLILAVLGVYMLAMPANTVSAAGVPFNTKYPYGPTSIAD
NQSEVTAMLKAEWEDWKSKRITSNGAGGYKRVQRDASTNYDTVSEGMGYG
LLLAVCFNEQALFDDLYRYVKSHFNGNGLMHWHIDANNNVTSHDGGDGAA
TDADEDIALALIFADKLWGSSGAINYGQEARTLINNLYNHCVEHGSYVLK
PGDRWGGSSVTNPSYFAPAWYKVYAQYTGDTRWNQVADKCYQIVEEVKKY
NNGTGLVPDWCTASGTPASGQSYDYKYDATRYGWRTAVDYSWEGDQRAKA
NCDMLTKFFARDGAKGIVDGYTIQGSKISNNHNASFIGPVAAASMTGYDL
NFAKELYRETVAVKDSEYYGYYGNSLRLLTLLYITGNFPNPLSDLSGQPT
PPSNPTPSLPPQVVYGDVNGDGNVNSTDLTMLKRYLLKSVTNINREAADV
NRDGAINSSDMTILKRYLIKSIPHLPY

or is substantially similar to SEQ ID NO: 44 or is an active fragment of SEQ ID NO: 44. In some embodiments, the polypeptide comprises a formula of X₁—Z₁, wherein Z₁ comprises an amino acid sequence having least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 44. In some embodiments, the polypeptide comprises a formula of X₁—Z₁, wherein Z₁ comprises an amino acid sequence of SEQ ID NO: 44.

In some embodiments, the polypeptide comprises a formula of X₁—Z₁, wherein Z₁ comprises an amino acid sequence having at least 70% identity to SEQ ID NO: 72:

(SEQ ID NO: 72)
VFTLEDFVGDWRQTAGYNLDQVLEQGGVSSLFQNLGVSVTPIQRIVLSGE
NGLKIDIHVIIPYEGLSGDQMGQIEKIFKVVYPVDDHHFKVILHYGTLVI
DGVTPNMIDYFGRPYEGIAVFDGKKITVTGTLWNGNKIIDERLINPDGSL
LFRVTINGVTGWRLCERILA

or is substantially similar to SEQ ID NO: 72 or is an active fragment of SEQ ID NO: 72. In some embodiments, the polypeptide comprises a formula of X₁—Z₁, wherein Z₁ comprises an amino acid sequence having least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 72. In some embodiments, the polypeptide comprises a formula of X₁—Z₁, wherein Z₁ comprises an amino acid sequence of SEQ ID NO: 72.

In any of the embodiments herein, the polypeptide comprises a formula of X₁—Z₁, wherein Z₁ is as provided for herein and may further comprise an affinity tag. The affinity tag may be utilized, for example, for protein purification or detection. The affinity tag may be utilized for any method known in the art for which affinity tags are utilized. Affinity tags are known in the art, and any such affinity tag may be utilized. Non-limiting examples of affinity tags that may be utilized include 6×HIS (SEQ ID NO: 45), FLAG, GST, MBP, a streptavidin peptide, GFP, and the like. In some embodiments, any peptide sequence that can be utilized for purification or detection may be utilized.

In some embodiments, the polypeptide comprises a formula of X₁—Z₁, wherein X₁ comprises an amino acid sequence of Formula I, and Z₁ comprises an amino acid sequence selected from the group comprising SEQ ID NO: 28, 29, 30, 31, 32, 33, 34, 35, 36, 37, 38, 39, 40, 41, 42, 43, 44, or 72. In some embodiments, the polypeptide comprises a formula of X₁—Z₁, wherein X₁ comprises an amino acid sequence selected from the group consisting of SEQ ID NO: 1, 2, 3, or 4, and Z₁ comprises an amino acid sequence selected from the group comprising SEQ ID NO: 28, 29, 30, 31, 32, 33, 34, 35, 36, 37, 38, 39, 40, 41, 42, 43, 44, or 72. In some embodiments, the components X₁ and Z₁ are fused directly. In some embodiments, the components X₁ and Z₁ are fused indirectly via, for example, a peptide linker as provided for herein. In some embodiments, the pre-protein signal peptide X₁ increases the secretion of the payload protein Z₁ as compared to native signal peptides.

In some embodiments, the polypeptide comprises a formula of X₁—Z₁, wherein X₁ comprises an amino acid sequence of SEQ ID NO: 1, and Z₁ comprises an amino acid sequence of SEQ ID NO: 28. In some embodiments, the polypeptide comprises a formula of X₁—Z₁, wherein X₁ comprises an amino acid sequence of SEQ ID NO: 2, and Z₁ comprises an amino acid sequence of SEQ ID NO: 28. In some embodiments, the polypeptide comprises a formula of X₁—Z₁, wherein X₁ comprises an amino acid sequence of SEQ ID NO: 3, and Z₁ comprises an amino acid sequence of SEQ ID NO: 28. In some embodiments, the polypeptide comprises a formula of X₁—Z₁, wherein X₁ comprises an amino acid sequence of SEQ ID NO: 4, and Z₁ comprises an amino acid sequence of SEQ ID NO: 28.

In some embodiments, the polypeptide comprises a formula of X₁—Z₁, wherein X₁ comprises an amino acid sequence of SEQ ID NO: 1, and Z₁ comprises an amino acid sequence of SEQ ID NO: 29. In some embodiments, the polypeptide comprises a formula of X₁—Z₁, wherein X₁ comprises an amino acid sequence of SEQ ID NO: 2, and Z₁ comprises an amino acid sequence of SEQ ID NO: 29. In some embodiments, the polypeptide comprises a formula of X₁—Z₁, wherein X₁ comprises an amino acid sequence of SEQ ID NO: 3, and Z₁ comprises an amino acid sequence of SEQ ID NO: 29. In some embodiments, the polypeptide comprises a formula of X₁—Z₁, wherein X₁ comprises an amino acid sequence of SEQ ID NO: 4, and Z₁ comprises an amino acid sequence of SEQ ID NO: 29.

In some embodiments, the polypeptide comprises a formula of X₁—Z₁, wherein X₁ comprises an amino acid sequence of SEQ ID NO: 1, and Z₁ comprises an amino acid sequence of SEQ ID NO: 30. In some embodiments, the polypeptide comprises a formula of X₁—Z₁, wherein X₁ comprises an amino acid sequence of SEQ ID NO: 2, and Z₁ comprises an amino acid sequence of SEQ ID NO: 30. In some embodiments, the polypeptide comprises a formula of X₁—Z₁, wherein X₁ comprises an amino acid sequence of SEQ ID NO: 3, and Z₁ comprises an amino acid sequence of SEQ ID NO: 30. In some embodiments, the polypeptide comprises a formula of X₁—Z₁, wherein X₁ comprises an amino acid sequence of SEQ ID NO: 4, and 7.1 comprises an amino acid sequence of SEQ ID NO: 30.

In some embodiments, the polypeptide comprises a formula of X₁—Z₁, wherein X₁ comprises an amino acid sequence of SEQ ID NO: 1, and Z₁ comprises an amino acid sequence of SEQ ID NO: 31. In some embodiments, the polypeptide comprises a formula of X₁—Z₁, wherein X₁ comprises an amino acid sequence of SEQ ID NO: 2, and Z₁ comprises an amino acid sequence of SEQ ID NO: 31. In some embodiments, the polypeptide comprises a formula of X₁—Z₁, wherein X₁ comprises an amino acid sequence of SEQ ID NO: 3, and Z₁ comprises an amino acid sequence of SEQ ID NO: 31. In some embodiments, the polypeptide comprises a formula of X₁—Z₁, wherein X₁ comprises an amino acid sequence of SEQ ID NO: 4, and Z₁ comprises an amino acid sequence of SEQ ID NO: 31.

In some embodiments, the polypeptide comprises a formula of X₁—Z₁, wherein X₁ comprises an amino acid sequence of SEQ ID NO: 1, and Z₁ comprises an amino acid sequence of SEQ ID NO: 32. In some embodiments, the polypeptide comprises a formula of X₁—Z₁, wherein X₁ comprises an amino acid sequence of SEQ ID NO: 2, and Z₁ comprises an amino acid sequence of SEQ ID NO: 32. In some embodiments, the polypeptide comprises a formula of X₁—Z₁, wherein X₁ comprises an amino acid sequence of SEQ ID NO: 3, and Z₁ comprises an amino acid sequence of SEQ ID NO: 32. In some embodiments, the polypeptide comprises a formula of X₁—Z₁, wherein X₁ comprises an amino acid sequence of SEQ ID NO: 4, and Z₁ comprises an amino acid sequence of SEQ ID NO: 32.

In some embodiments, the polypeptide comprises a formula of X₁—Z₁, wherein X₁ comprises an amino acid sequence of SEQ ID NO: 1, and Z₁ comprises an amino acid sequence of SEQ ID NO: 33. In some embodiments, the polypeptide comprises a formula of X₁—Z₁, wherein X₁ comprises an amino acid sequence of SEQ ID NO: 2, and Z₁ comprises an amino acid sequence of SEQ ID NO: 33. In some embodiments, the polypeptide comprises a formula of X₁—Z₁, wherein X₁ comprises an amino acid sequence of SEQ ID NO: 3, and Z₁ comprises an amino acid sequence of SEQ ID NO: 33. In some embodiments, the polypeptide comprises a formula of X₁—Z₁, wherein X₁ comprises an amino acid sequence of SEQ ID NO: 4, and Z₁ comprises an amino acid sequence of SEQ ID NO: 33.

In some embodiments, the polypeptide comprises a formula of X₁—Z₁, wherein X₁ comprises an amino acid sequence of SEQ ID NO: 1, and Z₁ comprises an amino acid sequence of SEQ ID NO: 34. In some embodiments, the polypeptide comprises a formula of X₁—Z₁, wherein X₁ comprises an amino acid sequence of SEQ ID NO: 2, and Z₁ comprises an amino acid sequence of SEQ ID NO: 34. In some embodiments, the polypeptide comprises a formula of X₁—Z₁, wherein X₁ comprises an amino acid sequence of SEQ ID NO: 3, and 7.1 comprises an amino acid sequence of SEQ ID NO: 34. In some embodiments, the polypeptide comprises a formula of X₁—Z₁, wherein X₁ comprises an amino acid sequence of SEQ ID NO: 4, and Z₁ comprises an amino acid sequence of SEQ ID NO: 34.

In some embodiments, the polypeptide comprises a formula of X₁—Z₁, wherein X₁ comprises an amino acid sequence of SEQ ID NO: 1, and Z₁ comprises an amino acid sequence of SEQ ID NO: 35. In some embodiments, the polypeptide comprises a formula of X₁—Z₁, wherein X₁ comprises an amino acid sequence of SEQ ID NO: 2, and Z₁ comprises an amino acid sequence of SEQ ID NO: 35. In some embodiments, the polypeptide comprises a formula of X₁—Z₁, wherein X₁ comprises an amino acid sequence of SEQ ID NO: 3, and Z₁ comprises an amino acid sequence of SEQ ID NO: 35. In some embodiments, the polypeptide comprises a formula of X₁—Z₁, wherein X₁ comprises an amino acid sequence of SEQ ID NO: 4, and Z₁ comprises an amino acid sequence of SEQ ID NO: 35.

In some embodiments, the polypeptide comprises a formula of X₁—Z₁, wherein X₁ comprises an amino acid sequence of SEQ ID NO: 1, and Z₁ comprises an amino acid sequence of SEQ ID NO: 36. In some embodiments, the polypeptide comprises a formula of X₁—Z₁, wherein X₁ comprises an amino acid sequence of SEQ ID NO: 2, and Z₁ comprises an amino acid sequence of SEQ ID NO: 36. In some embodiments, the polypeptide comprises a formula of X₁—Z₁, wherein X₁ comprises an amino acid sequence of SEQ ID NO: 3, and Z₁ comprises an amino acid sequence of SEQ ID NO: 36. In some embodiments, the polypeptide comprises a formula of X₁—Z₁, wherein X₁ comprises an amino acid sequence of SEQ ID NO: 4, and Z₁ comprises an amino acid sequence of SEQ ID NO: 36.

In some embodiments, the polypeptide comprises a formula of X₁—Z₁, wherein X₁ comprises an amino acid sequence of SEQ ID NO: 1, and Z₁ comprises an amino acid sequence of SEQ ID NO: 37. In some embodiments, the polypeptide comprises a formula of X₁—Z₁, wherein X₁ comprises an amino acid sequence of SEQ ID NO: 2, and Z₁ comprises an amino acid sequence of SEQ ID NO: 37. In some embodiments, the polypeptide comprises a formula of X₁—Z₁, wherein X₁ comprises an amino acid sequence of SEQ ID NO: 3, and Z₁ comprises an amino acid sequence of SEQ ID NO: 37. In some embodiments, the polypeptide comprises a formula of X₁—Z₁, wherein X₁ comprises an amino acid sequence of SEQ ID NO: 4, and Z₁ comprises an amino acid sequence of SEQ ID NO: 37.

In some embodiments, the polypeptide comprises a formula of X₁—Z₁, wherein X₁ comprises an amino acid sequence of SEQ ID NO: 1, and Z₁ comprises an amino acid sequence of SEQ ID NO: 38. In some embodiments, the polypeptide comprises a formula of X₁—Z₁, wherein X₁ comprises an amino acid sequence of SEQ ID NO: 2, and Z₁ comprises an amino acid sequence of SEQ ID NO: 38. In some embodiments, the polypeptide comprises a formula of X₁—Z₁, wherein X₁ comprises an amino acid sequence of SEQ ID NO: 3, and Z₁ comprises an amino acid sequence of SEQ ID NO: 38. In some embodiments, the polypeptide comprises a formula of X₁—Z₁, wherein X₁ comprises an amino acid sequence of SEQ ID NO: 4, and Z₁ comprises an amino acid sequence of SEQ ID NO: 38.

In some embodiments, the polypeptide comprises a formula of X₁—Z₁, wherein X₁ comprises an amino acid sequence of SEQ ID NO: 1, and Z₁ comprises an amino acid sequence of SEQ ID NO: 39. In some embodiments, the polypeptide comprises a formula of X₁—Z₁, wherein X₁ comprises an amino acid sequence of SEQ ID NO: 2, and Z₁ comprises an amino acid sequence of SEQ ID NO: 39. In some embodiments, the polypeptide comprises a formula of X₁—Z₁, wherein X₁ comprises an amino acid sequence of SEQ ID NO: 3, and Z₁ comprises an amino acid sequence of SEQ ID NO: 39. In some embodiments, the polypeptide comprises a formula of X₁—Z₁, wherein X₁ comprises an amino acid sequence of SEQ ID NO: 4, and 7.1 comprises an amino acid sequence of SEQ ID NO: 39.

In some embodiments, the polypeptide comprises a formula of X₁—Z₁, wherein X₁ comprises an amino acid sequence of SEQ ID NO: 1, and Z₁ comprises an amino acid sequence of SEQ ID NO: 40. In some embodiments, the polypeptide comprises a formula of X₁—Z₁, wherein X₁ comprises an amino acid sequence of SEQ ID NO: 2, and Z₁ comprises an amino acid sequence of SEQ ID NO: 40. In some embodiments, the polypeptide comprises a formula of X₁—Z₁, wherein X₁ comprises an amino acid sequence of SEQ ID NO: 3, and Z₁ comprises an amino acid sequence of SEQ ID NO: 40. In some embodiments, the polypeptide comprises a formula of X₁—Z₁, wherein X₁ comprises an amino acid sequence of SEQ ID NO: 4, and 7.1 comprises an amino acid sequence of SEQ ID NO: 40.

In some embodiments, the polypeptide comprises a formula of X₁—Z₁, wherein X₁ comprises an amino acid sequence of SEQ ID NO: 1, and Z₁ comprises an amino acid sequence of SEQ ID NO: 41. In some embodiments, the polypeptide comprises a formula of X₁—Z₁, wherein X₁ comprises an amino acid sequence of SEQ ID NO: 2, and Z₁ comprises an amino acid sequence of SEQ ID NO: 41. In some embodiments, the polypeptide comprises a formula of X₁—Z₁, wherein X₁ comprises an amino acid sequence of SEQ ID NO: 3, and Z₁ comprises an amino acid sequence of SEQ ID NO: 41. In some embodiments, the polypeptide comprises a formula of X₁—Z₁, wherein X₁ comprises an amino acid sequence of SEQ ID NO: 4, and Z₁ comprises an amino acid sequence of SEQ ID NO: 41.

In some embodiments, the polypeptide comprises a formula of X₁—Z₁, wherein X₁ comprises an amino acid sequence of SEQ ID NO: 1, and 71 comprises an amino acid sequence of SEQ ID NO: 42. In some embodiments, the polypeptide comprises a formula of X₁—Z₁, wherein X₁ comprises an amino acid sequence of SEQ ID NO: 2, and Z₁ comprises an amino acid sequence of SEQ ID NO: 42. In some embodiments, the polypeptide comprises a formula of X₁—Z₁, wherein X₁ comprises an amino acid sequence of SEQ ID NO: 3, and Z₁ comprises an amino acid sequence of SEQ ID NO: 42. In some embodiments, the polypeptide comprises a formula of X₁—Z₁, wherein X₁ comprises an amino acid sequence of SEQ ID NO: 4, and Z₁ comprises an amino acid sequence of SEQ ID NO: 42.

In some embodiments, the polypeptide comprises a formula of X₁—Z₁, wherein X₁ comprises an amino acid sequence of SEQ ID NO: 1, and Z₁ comprises an amino acid sequence of SEQ ID NO: 43. In some embodiments, the polypeptide comprises a formula of X₁—Z₁, wherein X₁ comprises an amino acid sequence of SEQ ID NO: 2, and Z₁ comprises an amino acid sequence of SEQ ID NO: 43. In some embodiments, the polypeptide comprises a formula of X₁-71, wherein X₁ comprises an amino acid sequence of SEQ ID NO: 3, and 71 comprises an amino acid sequence of SEQ ID NO: 43. In some embodiments, the polypeptide comprises a formula of X₁—Z₁, wherein X₁ comprises an amino acid sequence of SEQ ID NO: 4, and Z₁ comprises an amino acid sequence of SEQ ID NO: 43.

In some embodiments, the polypeptide comprises a formula of X₁—Z₁, wherein X₁ comprises an amino acid sequence of SEQ ID NO: 1, and Z₁ comprises an amino acid sequence of SEQ ID NO: 44. In some embodiments, the polypeptide comprises a formula of X₁—Z₁, wherein X₁ comprises an amino acid sequence of SEQ ID NO: 2, and Z₁ comprises an amino acid sequence of SEQ ID NO: 44. In some embodiments, the polypeptide comprises a formula of X₁—Z₁, wherein X₁ comprises an amino acid sequence of SEQ ID NO: 3, and 71 comprises an amino acid sequence of SEQ ID NO: 44. In some embodiments, the polypeptide comprises a formula of X₁—Z₁, wherein X₁ comprises an amino acid sequence of SEQ ID NO: 4, and Z₁ comprises an amino acid sequence of SEQ ID NO: 44.

In some embodiments, the polypeptide comprises a formula of X₁—Z₁, wherein X₁ comprises an amino acid sequence of SEQ ID NO: 1, and Z₁ comprises an amino acid sequence of SEQ ID NO: 72. In some embodiments, the polypeptide comprises a formula of X₁—Z₁, wherein X₁ comprises an amino acid sequence of SEQ ID NO: 2, and Z₁ comprises an amino acid sequence of SEQ ID NO: 72. In some embodiments, the polypeptide comprises a formula of X₁—Z₁, wherein X₁ comprises an amino acid sequence of SEQ ID NO: 3, and Z₁ comprises an amino acid sequence of SEQ ID NO: 72. In some embodiments, the polypeptide comprises a formula of X₁—Z₁, wherein X₁ comprises an amino acid sequence of SEQ ID NO: 4, and 7.1 comprises an amino acid sequence of SEQ ID NO: 72.

As provided for herein, in some embodiments, the polypeptide is a recombinant polypeptide. As provided for herein, in some embodiments, the recombinant polypeptide comprises a formula of X₁—Z₁, wherein X₁ is a pre-protein signal peptide and Z₁ is a payload protein. In some embodiments, the recombinant polypeptide comprises a formula of X₁—Z₁, wherein X₁ is a pre-protein signal peptide as provided for herein. In some embodiments, the recombinant polypeptide comprises a formula of X₁—Z₁, wherein Z₁ is a payload protein as provided for herein.

In some embodiments, a nucleic acid is provided. In some embodiments, the nucleic acid encodes for a polypeptide as provided for herein. In some embodiments, the polypeptide comprises a signal peptide and a pay load protein. In some embodiments, the signal peptide is as provided for herein. In some embodiments, the payload protein is as provided for herein. In some embodiments, the nucleic acid encodes for a recombinant polypeptide as provided for herein. In some embodiments, the recombinant polypeptide comprises a signal peptide and a payload protein. In some embodiments, the signal peptide is as provided for herein. In some embodiments, the payload protein is as provided for herein.

In some embodiments, a bacterium is provided. In some embodiments, the bacterium in an engineered bacterium. In some embodiments, the engineered bacterium comprises a heterologous nucleic acid molecule encoding for a polypeptide having a formula X₁—Z₁, wherein X₁ is a pre-protein signal peptide as provided for herein, and Z₁ is a payload protein. In some embodiments, the pre-protein signal peptide X₁ increases the secretion of the payload protein 71 as compared to native signal peptides.

In some embodiments, the engineered bacterium comprises a heterologous nucleic acid molecule encoding for a polypeptide having a formula of X₁—Z₁, wherein X₁ comprises an amino acid sequence of Formula I. In some embodiments, the engineered bacterium comprises a heterologous nucleic acid molecule encoding for a polypeptide having a formula of X₁—Z₁, wherein X₁ comprises an amino acid sequence having at last 70% identity to an amino acid sequence selected from the group consisting of SEQ ID NO: 1, 2, 3, or 4. In some embodiments, the engineered bacterium comprises a heterologous nucleic acid molecule encoding for a polypeptide having a formula of X₁—Z₁, wherein X₁ comprises an amino acid sequence having at least at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to an amino acid sequence selected from the group consisting of SEQ ID NO: 1, 2, 3, or 4. In some embodiments, the engineered bacterium comprises a heterologous nucleic acid molecule encoding for a polypeptide having a formula of X₁—Z₁, wherein X₁ comprises an amino acid sequence selected from the group consisting of SEQ ID NO: 1, 2, 3, and 4. In some embodiments, the engineered bacterium comprises a heterologous nucleic acid molecule encoding for a polypeptide having a formula of X₁—Z₁, wherein X₁ comprises an amino acid sequence of SEQ ID NO: 1. In some embodiments, the engineered bacterium comprises a heterologous nucleic acid molecule encoding for a polypeptide having a formula of X₁—Z₁, wherein X₁ comprises an amino acid sequence of SEQ ID NO: 2. In some embodiments, the engineered bacterium comprises a heterologous nucleic acid molecule encoding for a polypeptide having a formula of X₁—Z₁, wherein X₁ comprises an amino acid sequence of SEQ ID NO: 3. In some embodiments, the engineered bacterium comprises a heterologous nucleic acid molecule encoding for a polypeptide having a formula of X₁—Z₁, wherein X₁ comprises an amino acid sequence of SEQ ID NO: 4.

In some embodiments, the engineered bacterium comprises a heterologous nucleic acid molecule encoding for a polypeptide having a formula of X₁—Z₁, wherein Z₁ is any peptide or protein. In some embodiments, the engineered bacterium comprises a heterologous nucleic acid molecule encoding for a polypeptide having a formula of X₁—Z₁, wherein Z₁ is selected from the group including, but not limited to, an antiviral, insulin, an incretin, an enzyme, an enzyme inhibitor, a hormone, a cytokine, an antibody, a single domain antibody fragment, a single chain variable antibody fragment, an antimicrobial peptide, a mucosal protein, pesticide, bactericide herbicide, fungicide, nematicide, miticide, plant growth regulator, plant growth stimulator or fertilizer, a vaccine, a diagnostic protein, a feed conversion enzyme, a flavoring, a nutritional protein, venom peptides, endoglucanase, restriction enzymes, human growth hormone, insulin, Beta-lactamase, luciferase (such as, but not limited to, Renilla luciferase or NanoLuc luciferase), phytase, cellulase, chitinase, phosphatase, catalase, urokinase, tissue plasminogen activator, apolipoprotein, beta-glucosidases, hemicellulases, lignocellulose oxireductases, DNAses NADPH dehydrogenase, alcohol oxidase, pyruvate decarboxylase, formolase, alpha-ketoglutarate dehydrogenase, branched chain alpha-ketoacid decarboxylase, copper radical oxidase, galactose oxidase, glycerol oxidase, amine oxidase, glyoxalase, amino monoaxidase, ethylene glycol oxidase, alditol oxidase, or 2-oxoglutarate dehydrogenase. In some embodiments, the engineered bacterium comprises a heterologous nucleic acid molecule encoding for a polypeptide having a formula of X₁—Z₁, wherein Z₁ is selected from the group including, but not limited to, an enzyme (e.g., invertase, isomaltase, lactase, lysozyme, An-PEP), a growth factor (e.g., IGF1), insulin, an incretin (e.g., GLP-1, GLP-2, leptin, apelin, ghrelin, PYY, nesfatin), a cytokine, an antibody, an antimicrobial peptide), a mucosal protein (e.g., trefoil factor, Reg3 protein, superoxide dismutase), an agricultural product (e.g., pesticide, bactericide herbicide, fungicide, nematicide, miticide, plant growth regulator, plant growth stimulator, or fertilizer), a vaccine, a diagnostic protein, a feed conversion enzyme, a flavoring, or a nutritional protein. In some embodiments, the engineered bacterium comprises a heterologous nucleic acid molecule encoding for a polypeptide having a formula of X₁—Z₁, wherein Z₁ is selected from the group including, but not limited to, venom peptides, endoglucanase, restriction enzymes, human growth hormone, insulin, Beta-lactamase, luciferase (such as, but not limited to, Renilla luciferase or NanoLuc luciferase), phytase, cellulase, chitinase, phosphatase, catalase, urokinase, tissue plasminogen activator, apolipoprotein, NADPH dehydrogenase, alcohol oxidase, pyruvate decarboxylase, formolase, alpha-ketoglutarate dehydrogenase, branched chain alpha-ketoacid decarboxylase, copper radical oxidase, galactose oxidase, glycerol oxidase, amine oxidase, glyoxalase, amino monoaxidase, ethylene glycol oxidase, alditol oxidase, or 2-oxoglutarate dehydrogenase. In some embodiments, the engineered bacterium comprises a heterologous nucleic acid molecule encoding for a polypeptide having a formula of X₁—Z₁, wherein Z₁ is selected from the group including, but not limited to, amylases, alpha amylases, xylanases (e.g. endo-1,4-beta-xylanase), lichenases (e.g. beta glucanase), lipases (e.g. Candida antartica lipase B, Candida rugose lipase, LipA), pectinases (e.g. pectate trisaccharide lyase), and cellulases (e.g. endoglucanase A). The examples listed are provided for clarity only and are not meant to be limiting in any way. Thus, for example, the current disclosure is not limited to IGF-1 for “growth factor”, but rather encompasses and includes all growth factors known in the art.

In some embodiments, the engineered bacterium comprises a heterologous nucleic acid molecule encoding for a polypeptide having a formula of X₁—Z₁, wherein Z₁ comprises an amino acid sequence having at least 70% identity to an amino acid sequence selected from the group consisting of SEQ ID NO: 28, 29, 30, 31, 32, 33, 34, 35, 36, 37, 38, 39, 40, 41, 42, 43, 44, or 72. In some embodiments, the engineered bacterium comprises a heterologous nucleic acid molecule encoding for a polypeptide having a formula of X₁—Z₁, wherein Z₁ comprises an amino acid sequence having least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to an amino acid sequence selected from the group consisting of SEQ ID NO: 28, 29, 30, 31, 32, 33, 34, 35, 36, 37, 38, 39, 40, 41, 42, 43, 44, or 72. In some embodiments, the engineered bacterium comprises a heterologous nucleic acid molecule encoding for a polypeptide having a formula of X₁—Z₁, wherein Z₁ comprises an amino acid sequence selected from the group consisting of SEQ ID NO: 28, 29, 30, 31, 32, 33, 34, 35, 36, 37, 38, 39, 40, 41, 42, 43, 44, or 72.

In some embodiments, the engineered bacterium comprises a heterologous nucleic acid molecule encoding for a polypeptide having a formula of X₁—Z₁, wherein X₁ comprises an amino acid sequence of SEQ ID NO: 1, and Z₁ comprises an amino acid sequence of SEQ ID NO: 28. In some embodiments, the engineered bacterium comprises a heterologous nucleic acid molecule encoding for a polypeptide having a formula of X₁—Z₁, wherein X₁ comprises an amino acid sequence of SEQ ID NO: 2, and Z₁ comprises an amino acid sequence of SEQ ID NO: 28. In some embodiments, the engineered bacterium comprises a heterologous nucleic acid molecule encoding for a polypeptide having a formula of X₁—Z₁, wherein X₁ comprises an amino acid sequence of SEQ ID NO: 3, and Z₁ comprises an amino acid sequence of SEQ ID NO: 28. In some embodiments, the engineered bacterium comprises a heterologous nucleic acid molecule encoding for a polypeptide having a formula of X₁—Z₁, wherein X₁ comprises an amino acid sequence of SEQ ID NO: 4, and Z₁ comprises an amino acid sequence of SEQ ID NO: 28.

In some embodiments, the engineered bacterium comprises a heterologous nucleic acid molecule encoding for a polypeptide having a formula of X₁—Z₁, wherein X₁ comprises an amino acid sequence of SEQ ID NO: 1, and Z₁ comprises an amino acid sequence of SEQ ID NO: 29. In some embodiments, the engineered bacterium comprises a heterologous nucleic acid molecule encoding for a polypeptide having a formula of X₁—Z₁, wherein X₁ comprises an amino acid sequence of SEQ ID NO: 2, and 71 comprises an amino acid sequence of SEQ ID NO: 29. In some embodiments, the engineered bacterium comprises a heterologous nucleic acid molecule encoding for a polypeptide having a formula of X₁—Z₁, wherein X₁ comprises an amino acid sequence of SEQ ID NO: 3, and Z₁ comprises an amino acid sequence of SEQ ID NO: 29. In some embodiments, the engineered bacterium comprises a heterologous nucleic acid molecule encoding for a polypeptide having a formula of X₁—Z₁, wherein X₁ comprises an amino acid sequence of SEQ ID NO: 4, and Z₁ comprises an amino acid sequence of SEQ ID NO: 29.

In some embodiments, the engineered bacterium comprises a heterologous nucleic acid molecule encoding for a polypeptide having a formula of X₁—Z₁, wherein X₁ comprises an amino acid sequence of SEQ ID NO: 1, and Z₁ comprises an amino acid sequence of SEQ ID NO: 30. In some embodiments, the engineered bacterium comprises a heterologous nucleic acid molecule encoding for a polypeptide having a formula of X₁—Z₁, wherein X₁ comprises an amino acid sequence of SEQ ID NO: 2, and Z₁ comprises an amino acid sequence of SEQ ID NO: 30. In some embodiments, the engineered bacterium comprises a heterologous nucleic acid molecule encoding for a polypeptide having a formula of X₁—Z₁, wherein X₁ comprises an amino acid sequence of SEQ ID NO: 3, and Z₁ comprises an amino acid sequence of SEQ ID NO: 30. In some embodiments, the engineered bacterium comprises a heterologous nucleic acid molecule encoding for a polypeptide having a formula of X₁—Z₁, wherein X₁ comprises an amino acid sequence of SEQ ID NO: 4, and Z₁ comprises an amino acid sequence of SEQ ID NO: 30.

In some embodiments, the engineered bacterium comprises a heterologous nucleic acid molecule encoding for a polypeptide having a formula of X₁—Z₁, wherein X₁ comprises an amino acid sequence of SEQ ID NO: 1, and Z₁ comprises an amino acid sequence of SEQ ID NO: 31. In some embodiments, the engineered bacterium comprises a heterologous nucleic acid molecule encoding for a polypeptide having a formula of X₁—Z₁, wherein X₁ comprises an amino acid sequence of SEQ ID NO: 2, and Z₁ comprises an amino acid sequence of SEQ ID NO: 31. In some embodiments, the engineered bacterium comprises a heterologous nucleic acid molecule encoding for a polypeptide having a formula of X₁—Z₁, wherein X₁ comprises an amino acid sequence of SEQ ID NO: 3, and Z₁ comprises an amino acid sequence of SEQ ID NO: 31. In some embodiments, the engineered bacterium comprises a heterologous nucleic acid molecule encoding for a polypeptide having a formula of X₁—Z₁, wherein X₁ comprises an amino acid sequence of SEQ ID NO: 4, and Z₁ comprises an amino acid sequence of SEQ ID NO: 31.

In some embodiments, the engineered bacterium comprises a heterologous nucleic acid molecule encoding for a polypeptide having a formula of X₁—Z₁, wherein X₁ comprises an amino acid sequence of SEQ ID NO: 1, and Z₁ comprises an amino acid sequence of SEQ ID NO: 32. In some embodiments, the engineered bacterium comprises a heterologous nucleic acid molecule encoding for a polypeptide having a formula of X₁—Z₁, wherein X₁ comprises an amino acid sequence of SEQ ID NO: 2, and Z₁ comprises an amino acid sequence of SEQ ID NO: 32. In some embodiments, the engineered bacterium comprises a heterologous nucleic acid molecule encoding for a polypeptide having a formula of X₁—Z₁, wherein X₁ comprises an amino acid sequence of SEQ ID NO: 3, and Z₁ comprises an amino acid sequence of SEQ ID NO: 32. In some embodiments, the engineered bacterium comprises a heterologous nucleic acid molecule encoding for a polypeptide having a formula of X₁—Z₁, wherein X₁ comprises an amino acid sequence of SEQ ID NO: 4, and Z₁ comprises an amino acid sequence of SEQ ID NO: 32.

In some embodiments, the engineered bacterium comprises a heterologous nucleic acid molecule encoding for a polypeptide having a formula of X₁—Z₁, wherein X₁ comprises an amino acid sequence of SEQ ID NO: 1, and Z₁ comprises an amino acid sequence of SEQ ID NO: 33. In some embodiments, the engineered bacterium comprises a heterologous nucleic acid molecule encoding for a polypeptide having a formula of X₁—Z₁, wherein X₁ comprises an amino acid sequence of SEQ ID NO: 2, and Z₁ comprises an amino acid sequence of SEQ ID NO: 33. In some embodiments, the engineered bacterium comprises a heterologous nucleic acid molecule encoding for a polypeptide having a formula of X₁—Z₁, wherein X₁ comprises an amino acid sequence of SEQ ID NO: 3, and Z₁ comprises an amino acid sequence of SEQ ID NO: 33. In some embodiments, the engineered bacterium comprises a heterologous nucleic acid molecule encoding for a polypeptide having a formula of X₁—Z₁, wherein X₁ comprises an amino acid sequence of SEQ ID NO: 4, and Z₁ comprises an amino acid sequence of SEQ ID NO: 33.

In some embodiments, the engineered bacterium comprises a heterologous nucleic acid molecule encoding for a polypeptide having a formula of X₁—Z₁, wherein X₁ comprises an amino acid sequence of SEQ ID NO: 1, and Z₁ comprises an amino acid sequence of SEQ ID NO: 34. In some embodiments, the engineered bacterium comprises a heterologous nucleic acid molecule encoding for a polypeptide having a formula of X₁—Z₁, wherein X₁ comprises an amino acid sequence of SEQ ID NO: 2, and Z₁ comprises an amino acid sequence of SEQ ID NO: 34. In some embodiments, the engineered bacterium comprises a heterologous nucleic acid molecule encoding for a polypeptide having a formula of X₁—Z₁, wherein X₁ comprises an amino acid sequence of SEQ ID NO: 3, and 7.1 comprises an amino acid sequence of SEQ ID NO: 34. In some embodiments, the engineered bacterium comprises a heterologous nucleic acid molecule encoding for a polypeptide having a formula of X₁—Z₁, wherein X₁ comprises an amino acid sequence of SEQ ID NO: 4, and Z₁ comprises an amino acid sequence of SEQ ID NO: 34.

In some embodiments, the engineered bacterium comprises a heterologous nucleic acid molecule encoding for a polypeptide having a formula of X₁—Z₁, wherein X₁ comprises an amino acid sequence of SEQ ID NO: 1, and Z₁ comprises an amino acid sequence of SEQ ID NO: 35. In some embodiments, the engineered bacterium comprises a heterologous nucleic acid molecule encoding for a polypeptide having a formula of X₁—Z₁, wherein X₁ comprises an amino acid sequence of SEQ ID NO: 2, and Z₁ comprises an amino acid sequence of SEQ ID NO: 35. In some embodiments, the engineered bacterium comprises a heterologous nucleic acid molecule encoding for a polypeptide having a formula of X₁—Z₁, wherein X₁ comprises an amino acid sequence of SEQ ID NO: 3, and Z₁ comprises an amino acid sequence of SEQ ID NO: 35. In some embodiments, the engineered bacterium comprises a heterologous nucleic acid molecule encoding for a polypeptide having a formula of X₁—Z₁, wherein X₁ comprises an amino acid sequence of SEQ ID NO: 4, and Z₁ comprises an amino acid sequence of SEQ ID NO: 35.

In some embodiments, the engineered bacterium comprises a heterologous nucleic acid molecule encoding for a polypeptide having a formula of X₁—Z₁, wherein X₁ comprises an amino acid sequence of SEQ ID NO: 1, and Z₁ comprises an amino acid sequence of SEQ ID NO: 36. In some embodiments, the engineered bacterium comprises a heterologous nucleic acid molecule encoding for a polypeptide having a formula of X₁—Z₁, wherein X₁ comprises an amino acid sequence of SEQ ID NO: 2, and Z₁ comprises an amino acid sequence of SEQ ID NO: 36. In some embodiments, the engineered bacterium comprises a heterologous nucleic acid molecule encoding for a polypeptide having a formula of X₁—Z₁, wherein X₁ comprises an amino acid sequence of SEQ ID NO: 3, and Z₁ comprises an amino acid sequence of SEQ ID NO: 36. In some embodiments, the engineered bacterium comprises a heterologous nucleic acid molecule encoding for a polypeptide having a formula of X₁—Z₁, wherein X₁ comprises an amino acid sequence of SEQ ID NO: 4, and Z₁ comprises an amino acid sequence of SEQ ID NO: 36.

In some embodiments, the engineered bacterium comprises a heterologous nucleic acid molecule encoding for a polypeptide having a formula of X₁—Z₁, wherein X₁ comprises an amino acid sequence of SEQ ID NO: 1, and Z₁ comprises an amino acid sequence of SEQ ID NO: 37. In some embodiments, the engineered bacterium comprises a heterologous nucleic acid molecule encoding for a polypeptide having a formula of X₁—Z₁, wherein X₁ comprises an amino acid sequence of SEQ ID NO: 2, and Z₁ comprises an amino acid sequence of SEQ ID NO: 37. In some embodiments, the engineered bacterium comprises a heterologous nucleic acid molecule encoding for a polypeptide having a formula of X₁—Z₁, wherein X₁ comprises an amino acid sequence of SEQ ID NO: 3, and Z₁ comprises an amino acid sequence of SEQ ID NO: 37. In some embodiments, the engineered bacterium comprises a heterologous nucleic acid molecule encoding for a polypeptide having a formula of X₁—Z₁, wherein X₁ comprises an amino acid sequence of SEQ ID NO: 4, and Z₁ comprises an amino acid sequence of SEQ ID NO: 37.

In some embodiments, the engineered bacterium comprises a heterologous nucleic acid molecule encoding for a polypeptide having a formula of X₁—Z₁, wherein X₁ comprises an amino acid sequence of SEQ ID NO: 1, and 7.1 comprises an amino acid sequence of SEQ ID NO: 38. In some embodiments, the engineered bacterium comprises a heterologous nucleic acid molecule encoding for a polypeptide having a formula of X₁—Z₁, wherein X₁ comprises an amino acid sequence of SEQ ID NO: 2, and Z₁ comprises an amino acid sequence of SEQ ID NO: 38. In some embodiments, the engineered bacterium comprises a heterologous nucleic acid molecule encoding for a polypeptide having a formula of X₁—Z₁, wherein X₁ comprises an amino acid sequence of SEQ ID NO: 3, and Z₁ comprises an amino acid sequence of SEQ ID NO: 38. In some embodiments, the engineered bacterium comprises a heterologous nucleic acid molecule encoding for a polypeptide having a formula of X₁—Z₁, wherein X₁ comprises an amino acid sequence of SEQ ID NO: 4, and Z₁ comprises an amino acid sequence of SEQ ID NO: 38.

In some embodiments, the engineered bacterium comprises a heterologous nucleic acid molecule encoding for a polypeptide having a formula of X₁—Z₁, wherein X₁ comprises an amino acid sequence of SEQ ID NO: 1, and 71 comprises an amino acid sequence of SEQ ID NO: 39. In some embodiments, the engineered bacterium comprises a heterologous nucleic acid molecule encoding for a polypeptide having a formula of X₁—Z₁, wherein X₁ comprises an amino acid sequence of SEQ ID NO: 2, and Z₁ comprises an amino acid sequence of SEQ ID NO: 39. In some embodiments, the engineered bacterium comprises a heterologous nucleic acid molecule encoding for a polypeptide having a formula of X₁—Z₁, wherein X₁ comprises an amino acid sequence of SEQ ID NO: 3, and Z₁ comprises an amino acid sequence of SEQ ID NO: 39. In some embodiments, the engineered bacterium comprises a heterologous nucleic acid molecule encoding for a polypeptide having a formula of X₁—Z₁, wherein X₁ comprises an amino acid sequence of SEQ ID NO: 4, and 71 comprises an amino acid sequence of SEQ ID NO: 39.

In some embodiments, the engineered bacterium comprises a heterologous nucleic acid molecule encoding for a polypeptide having a formula of X₁—Z₁, wherein X₁ comprises an amino acid sequence of SEQ ID NO: 1, and Z₁ comprises an amino acid sequence of SEQ ID NO: 40. In some embodiments, the engineered bacterium comprises a heterologous nucleic acid molecule encoding for a polypeptide having a formula of X₁—Z₁, wherein X₁ comprises an amino acid sequence of SEQ ID NO: 2, and Z₁ comprises an amino acid sequence of SEQ ID NO: 40. In some embodiments, the engineered bacterium comprises a heterologous nucleic acid molecule encoding for a polypeptide having a formula of X₁—Z₁, wherein X₁ comprises an amino acid sequence of SEQ ID NO: 3, and Z₁ comprises an amino acid sequence of SEQ ID NO: 40. In some embodiments, the engineered bacterium comprises a heterologous nucleic acid molecule encoding for a polypeptide having a formula of X₁—Z₁, wherein X₁ comprises an amino acid sequence of SEQ ID NO: 4, and Z₁ comprises an amino acid sequence of SEQ ID NO: 40.

In some embodiments, the engineered bacterium comprises a heterologous nucleic acid molecule encoding for a polypeptide having a formula of X₁—Z₁, wherein X₁ comprises an amino acid sequence of SEQ ID NO: 1, and Z₁ comprises an amino acid sequence of SEQ ID NO: 41. In some embodiments, the engineered bacterium comprises a heterologous nucleic acid molecule encoding for a polypeptide having a formula of X₁—Z₁, wherein X₁ comprises an amino acid sequence of SEQ ID NO: 2, and Z₁ comprises an amino acid sequence of SEQ ID NO: 41. In some embodiments, the engineered bacterium comprises a heterologous nucleic acid molecule encoding for a polypeptide having a formula of X₁—Z₁, wherein X₁ comprises an amino acid sequence of SEQ ID NO: 3, and Z₁ comprises an amino acid sequence of SEQ ID NO: 41. In some embodiments, the engineered bacterium comprises a heterologous nucleic acid molecule encoding for a polypeptide having a formula of X₁—Z₁, wherein X₁ comprises an amino acid sequence of SEQ ID NO: 4, and Z₁ comprises an amino acid sequence of SEQ ID NO: 41.

In some embodiments, the engineered bacterium comprises a heterologous nucleic acid molecule encoding for a polypeptide having a formula of X₁—Z₁, wherein X₁ comprises an amino acid sequence of SEQ ID NO: 1, and Z₁ comprises an amino acid sequence of SEQ ID NO: 42. In some embodiments, the engineered bacterium comprises a heterologous nucleic acid molecule encoding for a polypeptide having a formula of X₁—Z₁, wherein X₁ comprises an amino acid sequence of SEQ ID NO: 2, and Z₁ comprises an amino acid sequence of SEQ ID NO: 42. In some embodiments, the engineered bacterium comprises a heterologous nucleic acid molecule encoding for a polypeptide having a formula of X₁—Z₁, wherein X₁ comprises an amino acid sequence of SEQ ID NO: 3, and Z₁ comprises an amino acid sequence of SEQ ID NO: 42. In some embodiments, the engineered bacterium comprises a heterologous nucleic acid molecule encoding for a polypeptide having a formula of X₁—Z₁, wherein X₁ comprises an amino acid sequence of SEQ ID NO: 4, and Z₁ comprises an amino acid sequence of SEQ ID NO: 42.

In some embodiments, the engineered bacterium comprises a heterologous nucleic acid molecule encoding for a polypeptide having a formula of X₁—Z₁, wherein X₁ comprises an amino acid sequence of SEQ ID NO: 1, and Z₁ comprises an amino acid sequence of SEQ ID NO: 43. In some embodiments, the engineered bacterium comprises a heterologous nucleic acid molecule encoding for a polypeptide having a formula of X₁—Z₁, wherein X₁ comprises an amino acid sequence of SEQ ID NO: 2, and 7.1 comprises an amino acid sequence of SEQ ID NO: 43. In some embodiments, the engineered bacterium comprises a heterologous nucleic acid molecule encoding for a polypeptide having a formula of X₁—Z₁, wherein X₁ comprises an amino acid sequence of SEQ ID NO: 3, and Z₁ comprises an amino acid sequence of SEQ ID NO: 43. In some embodiments, the engineered bacterium comprises a heterologous nucleic acid molecule encoding for a polypeptide having a formula of X₁—Z₁, wherein X₁ comprises an amino acid sequence of SEQ ID NO: 4, and Z₁ comprises an amino acid sequence of SEQ ID NO: 43.

In some embodiments, the engineered bacterium comprises a heterologous nucleic acid molecule encoding for a polypeptide having a formula of X₁—Z₁, wherein X₁ comprises an amino acid sequence of SEQ ID NO: 1, and Z₁ comprises an amino acid sequence of SEQ ID NO: 44. In some embodiments, the engineered bacterium comprises a heterologous nucleic acid molecule encoding for a polypeptide having a formula of X₁—Z₁, wherein X₁ comprises an amino acid sequence of SEQ ID NO: 2, and Z₁ comprises an amino acid sequence of SEQ ID NO: 44. In some embodiments, the engineered bacterium comprises a heterologous nucleic acid molecule encoding for a polypeptide having a formula of X₁—Z₁, wherein X₁ comprises an amino acid sequence of SEQ ID NO: 3, and Z₁ comprises an amino acid sequence of SEQ ID NO: 44. In some embodiments, the engineered bacterium comprises a heterologous nucleic acid molecule encoding for a polypeptide having a formula of X₁—Z₁, wherein X₁ comprises an amino acid sequence of SEQ ID NO: 4, and Z₁ comprises an amino acid sequence of SEQ ID NO: 44.

In some embodiments, the engineered bacterium comprises a heterologous nucleic acid molecule encoding for a polypeptide having a formula of X₁—Z₁, wherein X₁ comprises an amino acid sequence of SEQ ID NO: 1, and Z₁ comprises an amino acid sequence of SEQ ID NO: 72. In some embodiments, the engineered bacterium comprises a heterologous nucleic acid molecule encoding for a polypeptide having a formula of X₁—Z₁, wherein X₁ comprises an amino acid sequence of SEQ ID NO: 2, and Z₁ comprises an amino acid sequence of SEQ ID NO: 72. In some embodiments, the engineered bacterium comprises a heterologous nucleic acid molecule encoding for a polypeptide having a formula of X₁—Z₁, wherein X₁ comprises an amino acid sequence of SEQ ID NO: 3, and Z₁ comprises an amino acid sequence of SEQ ID NO: 72. In some embodiments, the engineered bacterium comprises a heterologous nucleic acid molecule encoding for a polypeptide having a formula of X₁—Z₁, wherein X₁ comprises an amino acid sequence of SEQ ID NO: 4, and Z₁ comprises an amino acid sequence of SEQ ID NO: 72.

In some embodiments, the engineered bacterium comprises a heterologous nucleic acid molecule encoding for a polypeptide having a formula of X₁—Z₁, wherein the components X₁ and Z₁ are fused directly. In some embodiments, the engineered bacterium comprises a heterologous nucleic acid molecule encoding for a polypeptide having a formula of X₁—Z₁, wherein the components X₁ and Z₁ are fused indirectly, via, for example, a peptide linker. Suitable peptide linkers are known in the art and any such linker may be utilized. In some embodiments, the linker is a flexible peptide linker. In some embodiments, the linker is a non-cleavable peptide linker. In some embodiments the linker is a cleavable peptide linker. Non-limiting examples of linkers are provided in the following table:

TABLE 11

| Type | Sequence |
|---|---|
| Flexible | GGGGS (SEQ ID NO: 46) |
| Flexible | (GGGGS)₃ (SEQ ID NO: 47) |
| Flexible | (GGGGS)ₙ (n = 1, 2, 3, 4) (SEQ ID |
| | NO: 48) |
| Flexible | (Gly)₈ (SEQ ID NO: 49) |
| Flexible | (Gly)₆ (SEQ ID NO: 50) |
| Rigid | (EAAAK)₃ (SEQ ID NO: 51) |
| Rigid | (EAAK)ₙ (n = 1-3) (SEQ ID NO: 52) |
| Rigid | A(EAAAK)₄ALEA(EAAAK)₄A |
| | (SEQ ID NO: 53) |
| Rigid | AEAAAKEAAAKA (SEQ ID NO: |
| | 54) |
| Rigid | PAPAP (SEQ ID NO: 55) |
| Rigid | (Ala-Pro)ₙ (10-34 aa) (SEQ ID NO: |
| | 56) |
| Cleavable | Disulfide |
| Cleavable | VSQTSKLTRAETVFPDV (SEQ ID |
| | NO: 57) |
| Cleavable | PLGLWA (SEQ ID NO: 58) |
| Cleavable | RVLAEA (SEQ ID NO: 59) |
| Cleavable | EDVVCCSMSY (SEQ ID NO: 60) |
| Cleavable | GGIEGRGS (SEQ ID NO: 61) |
| Cleavable | TRHRQPRGWE (SEQ ID NO: 62) |
| Cleavable | AGNRVRRSVG (SEQ ID NO: 63) |
| Cleavable | RRRRRRRRR (SEQ ID NO: 64) |
| Cleavable | GFLG (SEQ ID NO: 65) |
| Dipeptide | LE |

Synthetic Pre-Protein Signal Peptides and their Use in Escherichia Bacteria

In some embodiments, a synthetic pre-protein signal peptide is provided. In some embodiments, the pre-protein signal peptide may be fused directly or indirectly to a payload protein. In some embodiments, the pre-protein signal peptide is fused directly to the payload protein. In some embodiments, the pre-protein signal peptide is fused indirectly to the payload protein via, for example, a peptide linker. In some embodiments, the linker is a peptide linker as provided for herein. In some embodiments, fusion of the pre-protein signal peptide to the payload protein facilitates secretion of the payload protein from Escherichia bacteria. In some embodiments, any Escherichia bacteria may be used. In some embodiments, the Escherichia bacteria is as provided for herein. In some embodiments, the Escherichia bacteria is selected from the group including, but not limited to, E. albertii, E. fergusonii, E. hermannii, E. marmotae, and E. coli. In some embodiments, the Escherichia is E. coli. In some embodiments, the Escherichia bacteria may be genetically modified with a nucleic acid encoding for expression of a recombinant fusion protein. In some embodiments, the fusion protein comprises a synthetic pre-protein signal peptide fused either directly or indirectly to a payload protein. In some embodiments, the synthetic pre-protein is fused directly to the payload protein. In some embodiments, the pre-protein is fused indirectly to the payload protein via, for example, a peptide linker as provided for herein. In some embodiments, the synthetic pre-protein signal peptide comprises an amino acid sequence represented by SEQ ID NO: 1, SEQ ID NO: 2, SEQ ID NO: 3, SEQ ID NO: 4, or any amino acid sequence represented by Formula I. In some embodiments, the synthetic pre-protein signal peptide comprises an amino acid sequence of represented by Formula I. In some embodiments, the synthetic pre-protein signal peptide comprises an amino acid sequence of SEQ ID NO: 1. In some embodiments, the synthetic pre-protein signal peptide comprises an amino acid sequence of SEQ ID NO: 2. In some embodiments, the synthetic pre-protein signal peptide comprises an amino acid sequence of SEQ ID NO: 3. In some embodiments, the synthetic pre-protein signal peptide comprises an amino acid sequence of SEQ ID NO: 4. In some embodiments, the nucleic acid encoding for a peptide comprising an amino acid sequence represented by SEQ ID NO: 1, 2, 3, 4, or Formula I may be any nucleic acid sequence that encodes for such sequences. In some embodiments, the nucleic acid sequence encoding for the amino acid sequence of SEQ ID NO: 1 comprises a nucleic acid sequence of SEQ ID NO: 5. In some embodiments, the nucleic acid sequence encoding for an amino acid sequence of SEQ ID NO: 2 comprises a nucleic acid sequence of SEQ ID NO: 6. In some embodiments, the nucleic acid sequence encoding for an amino acid sequence of SEQ ID NO: 3 comprises a nucleic acid sequence of SEQ ID NO: 7. In some embodiments, the nucleic acid sequence encoding for an amino acid sequence of SEQ ID NO: 4 comprises a nucleic acid sequence of SEQ ID NO: 8. It should be understood that nucleic acid sequences embodied by SEQ ID NO: 5, SEQ ID NO: 6, SEQ ID NO: 7, and SEQ ID NO: 8 (as well as other nucleic acid sequences presented herein) are exemplary and are not intended to be limiting in any way. In some embodiments, the nucleic acid sequence is substantially similar to SEQ ID NO: 5, SEQ ID NO: 6, SEQ ID NO: 7, or SEQ ID NO: 8. In some embodiments, the nucleic acid comprises a sequence having at least 60% identity to SEQ ID NO: 5, SEQ ID NO: 6, SEQ ID NO: 7, or SEQ ID NO: 8. In some embodiments, the nucleic acid comprises a sequence having at least 65%, 70%, 75%, 80%, 85%, 86%, 87%, 88%, 89%, 90%, 91%, 92%, 93%, 94%, 95%, 96%, 97%, 98%, or 99% identity to SEQ ID NO: 5, SEQ ID NO: 6, SEQ ID NO: 7, or SEQ ID NO: 8. In some embodiments, the nucleic acid comprises a sequence identical to SEQ ID NO: 5, SEQ ID NO: 6, SEQ ID NO: 7, or SEQ ID NO: 8. Due to the degenerate nature of codons, other nucleic acid molecules can be used. In some embodiments, the nucleic acid molecule is codon optimized for expression in a bacterial system. In some embodiments, the nucleic acid molecule is codon optimized for expression in a eukaryotic system or cell. In some embodiments, the nucleic acid molecule is a DNA or RNA molecule that encodes a polypeptide as provided for herein. In some embodiments, the RNA molecule is a mRNA molecule. One who is skilled in the art will understand how to develop a suitable nucleotide sequence that will induce expression of a synthetic signal peptide comprising an amino acid sequence represented by Formula I. For example. Table 12 below provides various DNA codons that encode each amino acid.

TABLE 12

| | Amino | | Amino |
|---|---|---|---|
| DNA Codon | Acid | DNA Codon | Acid |
| GCT, GCC, GCA, GCG | A | TAA, TTG, CTT, | L |
| | | CTC, CTA, CTG | |
| CGT, CGC, CGA, CGG, | R | AAA, AAG | K |
| AGA, AGG | | | |
| AAT, AAC | N | ATG | M |
| GAT, GAC | D | TTT, TTC | F |
| TGT, TGC | C | CCT, CCC, CA, CCG | P |
| CAA, CAG | Q | TCT, TCC, TCA, | S |
| | | TCG, AGT, AGC | |
| GAA, GAG | E | ACT, ACC, ACA, ACG | T |
| GGT, GGC, GGA, GGG | G | TGG | W |
| CAT, CAC | H | TAT, TAC | Y |
| ATT, ATC, ATA | I | GTT, GTC, GTA, GTG | V |
| ATG | START | TAA, TGA, TAG | STOP |

A recombinant polypeptide comprising a synthetic pre-protein signal peptide comprising an amino acid sequence represented by Formula I or SEQ ID NO: 1, 2, 3, or 4 and a payload protein may be more readily secreted by the bacteria in which it is produced. The secretion may be to the culture media of the bacteria, or it may be to an extra cytoplasmic compartment, such as the periplasm. Accordingly, in some embodiments, a method of producing a payload protein with Escherichia bacteria is provided, the method comprising providing a nucleic acid encoding a recombinant polypeptide comprising a payload protein and a synthetic signal peptide; genetically modifying the Escherichia bacteria with the nucleic acid, thereby generating engineered bacteria; and culturing the bacteria under conditions to produce the recombinant polypeptide. In some embodiments, the synthetic signal peptide is fused directly or indirectly to the payload protein. In some embodiments, the signal peptide is fused directly to the payload protein. In some embodiments, the signal peptide is fused indirectly to the payload protein via, for example, a peptide linker as provided for herein. In some embodiments, the synthetic signal peptide is a pre-protein signal peptide. In some embodiments, the pre-protein signal peptide comprises an amino acid sequence represented by Formula I or SEQ ID NO: 1, 2, 3, or 4, as provided for herein. In some embodiments, the synthetic pre-protein signal peptide comprises an amino acid sequence of represented by Formula I. In some embodiments, the synthetic pre-protein signal peptide comprises an amino acid sequence of SEQ ID NO: 1. In some embodiments, the synthetic pre-protein signal peptide comprises an amino acid sequence of SEQ ID NO: 2. In some embodiments, the synthetic pre-protein signal peptide comprises an amino acid sequence of SEQ ID NO: 3. In some embodiments, the synthetic pre-protein signal peptide comprises an amino acid sequence of SEQ ID NO: 4. In some embodiments, any Escherichia bacteria may be used. In some embodiments, the Escherichia bacteria is selected from a Escherichia bacteria as provided for herein. In some embodiments, the Escherichia bacteria is selected from the group including, but not limited to E. albertii, E. fergusonii, E. hermannii, E. marmotae, and E. coli. In some embodiments, the Escherichia bacteria is E. coli. In some embodiments, the nucleic acid encoding the amino acid sequence represented by SEQ ID NO: 1 is as provided for herein. In some embodiments, the nucleic acid encoding the amino acid sequence represented by SEQ ID NO: 1 is represented by SEQ ID NO: 5. In some embodiments, the nucleic acid encoding the amino acid sequence represented by SEQ ID NO: 2 is as provided for herein. In some embodiments, the nucleic acid encoding the amino acid sequence represented by SEQ ID NO: 2 is represented by SEQ ID NO: 6. In some embodiments, the nucleic acid encoding the amino acid sequence represented by SEQ ID NO: 3 is as provided for herein. In some embodiments, the nucleic acid encoding the amino acid sequence represented by SEQ ID NO: 3 is represented by SEQ ID NO: 7. In some embodiments, the nucleic acid encoding the amino acid sequence represented by SEQ ID NO: 4 is as provided for herein. In some embodiments, the nucleic acid encoding the amino acid sequence represented by SEQ ID NO: 4 is represented by SEQ ID NO: 8.

In some embodiments, a method of increasing secretion of a payload protein from Escherichia bacteria is provided. Secretion, in the context of the present disclosure, encompasses both secretion to the culture media of the bacteria and secretion to an extra cytoplasmic compartment, such as the periplasm. In some embodiments, the secretion is to the culture media. In some embodiments, the secretion is to the periplasm. In some embodiments, the method comprises providing a nucleic acid encoding a recombinant polypeptide comprising a payload protein and a synthetic pre-protein signal peptide; genetically modifying the Escherichia bacteria with the nucleic acid, thereby generating an engineered bacteria, and culturing the engineered bacteria under effective conditions to secrete an increased amount of payload protein to the culture media, to the periplasm, or a combination thereof, when compared to the amount of payload protein secreted by Escherichia bacteria using a recombinant fusion protein comprising the payload protein and a known signal peptide. The known signal peptide may be any known signal peptide. In some embodiments, the known signal peptide is derived from amylase proteins (e.g., SEQ ID NO: 13 or SEQ ID NO: 14). In some embodiments, the known signal peptide comprises an amino acid sequence of SEQ ID NO: 15. In some embodiments, the known signal peptide comprises an amino acid sequence of SEQ ID NO: 66. In some embodiments, the known signal peptide comprises an amino acid sequence of SEQ ID NO: 68. In some embodiments, the known signal peptide comprises an amino acid sequence of SEQ ID NO: 70. In some embodiments, the bacteria is a bacteria as provided for herein. In some embodiments, the bacteria is genetically modified as provided for herein. In some embodiments, the recombinant polypeptide comprises a formula X₁—Z₁, wherein X₁ is a pre-protein signal peptide as provided for herein, and Z₁ is a payload protein. In some embodiments, X₁ comprises an amino acid sequence represented by Formula I or SEQ ID NO: 1, SEQ ID NO: 2, SEQ ID NO: 3, or SEQ ID NO: 4. In some embodiments, X₁ comprises an amino acid sequence of represented by Formula I. In some embodiments, X₁ comprises an amino acid sequence of SEQ ID NO: 1. In some embodiments, X₁ comprises an amino acid sequence of SEQ ID NO: 2. In some embodiments, X₁ comprises an amino acid sequence of SEQ ID NO: 3. In some embodiments, X₁ comprises an amino acid sequence of SEQ ID NO: 4. In some embodiments, X₁ is fused directly or indirectly to Z₁. In some embodiments, X₁ is fused directly to Z₁. In some embodiments, X₁ is fused indirectly to Z₁ via, for example, a linker peptide as provided for herein. In some embodiments, any Escherichia bacteria may be used. In some embodiments, the Escherichia is an Escherichia as provided for herein. In some embodiments, the Escherichia is selected from the group including, but not limited to, E. albertii, E. fergusonii, E. hermannii, E. marmotae, and E. coli. In some embodiments, the Escherichia is E. coli.

In some embodiments, engineered Escherichia bacteria genetically modified with a nucleic acid are provided. In some embodiments, the nucleic acid encodes for the expression of a recombinant polypeptide comprising a synthetic pre-protein signal peptide fused directly or indirectly to a payload protein. In some embodiments, the recombinant polypeptide comprises a formula of X₁—Z₁, wherein X₁ is a pre-protein signal peptide as provided for herein, and Z₁ is a payload protein. In some embodiments, the pre-protein signal peptide is fused directly to the payload protein. In some embodiments, the pre-protein signal peptide is fused indirectly to the payload protein via, for example, a linker peptide as provided for herein. In some embodiments, X₁ comprises an amino acid sequence selected from the group consisting of Formula I, SEQ ID NO: 1, SEQ ID NO: 2, SEQ ID NO: 3, and SEQ ID NO: 4. In some embodiments, X₁ comprises an amino acid sequence of represented by Formula I. In some embodiments, X₁ comprises an amino acid sequence of SEQ ID NO: 1. In some embodiments, X₁ comprises an amino acid sequence of SEQ ID NO: 2. In some embodiments, X₁ comprises an amino acid sequence of SEQ ID NO: 3. In some embodiments, X₁ comprises an amino acid sequence of SEQ ID NO: 4. In some embodiments, any Escherichia bacteria may be used. In some embodiments, the Escherichia is a Escherichia as provided for herein. In some embodiments, the Escherichia is selected from the group including but not limited to, E. albertii, E. fergusonii, E. hermannii, E. marmotae, and E. coli. In some embodiments, the Escherichia is E. coli. In some embodiments, the nucleic acid sequence comprises any nucleic acid sequence encoding for the pre-protein signal peptides as provided for herein. In some embodiments, the nucleic acid encoding the amino acid sequence of SEQ ID NO: 1 is SEQ ID NO: 5. In some embodiments, the nucleic acid encoding the amino acid sequence of SEQ ID NO: 2 is SEQ ID NO: 6. In some embodiments, the nucleic acid encoding the amino acid sequence of SEQ ID NO: 3 is SEQ ID NO: 7. In some embodiments, the nucleic acid encoding the amino acid sequence of SEQ ID NO: 4 is SEQ ID NO: 8.

In some embodiments, Z₁ is any peptide or protein. In some embodiments, the Z₁ is selected from the group including, but not limited to, an antiviral, insulin, an incretin, an enzyme, an enzyme inhibitor, a hormone, a cytokine, an antibody, a single domain antibody fragment, a single chain variable antibody fragment, an antimicrobial peptide, a mucosal protein, pesticide, bactericide herbicide, fungicide, nematicide, miticide, plant growth regulator, plant growth stimulator or fertilizer, a vaccine, a diagnostic protein, a feed conversion enzyme, a flavoring, a nutritional protein, venom peptides, endoglucanase, restriction enzymes, human growth hormone, insulin, Beta-lactamase, luciferase (such as, but not limited to, Renilla luciferase or NanoLuc luciferase), phytase, cellulase, chitinase, phosphatase, catalase, urokinase, tissue plasminogen activator, apolipoprotein, beta-glucosidases, hemicellulases, lignocellulose oxireductases, DNAses NADPH dehydrogenase, alcohol oxidase, pyruvate decarboxylase, formolase, alpha-ketoglutarate dehydrogenase, branched chain alpha-ketoacid decarboxylase, copper radical oxidase, galactose oxidase, glycerol oxidase, amine oxidase, glyoxalase, amino monoaxidase, ethylene glycol oxidase, alditol oxidase, or 2-oxoglutarate dehydrogenase. In some embodiments, the Z₁ is selected from the group including, but not limited to, an enzyme (e.g., invertase, isomaltase, lactase, lysozyme. An-PEP), a growth factor (e.g., IGF1), insulin, an incretin (e.g., GLP-1, GLP-2, leptin, apelin, ghrelin, PYY, nesfatin), a cytokine, an antibody, an antimicrobial peptide), a mucosal protein (e.g., trefoil factor, Reg3 protein, superoxide dismutase), an agricultural product (e.g., pesticide, bactericide herbicide, fungicide, nematicide, miticide, plant growth regulator, plant growth stimulator, or fertilizer), a vaccine, a diagnostic protein, a feed conversion enzyme, a flavoring, or a nutritional protein. In some embodiments, Z₁ is selected from the group including, but not limited to, venom peptides, endoglucanase, restriction enzymes, human growth hormone, insulin, Beta-lactamase, luciferase (such as, but not limited to, Renilla luciferase or NanoLuc luciferase), phytase, cellulase, chitinase, phosphatase, catalase, urokinase, tissue plasminogen activator, apolipoprotein, NADPH dehydrogenase, alcohol oxidase, pyruvate decarboxylase, formolase, alpha-ketoglutarate dehydrogenase, branched chain alpha-ketoacid decarboxylase, copper radical oxidase, galactose oxidase, glycerol oxidase, amine oxidase, glyoxalase, amino monoaxidase, ethylene glycol oxidase, alditol oxidase, or 2-oxoglutarate dehydrogenase. In some embodiments, Z₁ is selected from the group including, but not limited to, amylases, alpha amylases, xylanases (e.g. endo-1,4-beta-xylanase), lichenases (e.g. beta glucanase), lipases (e.g. Candida antartica lipase B, Candida rugose lipase, LipA), pectinases (e.g. pectate trisaccharide lyase), and cellulases (e.g. endoglucanase A). The examples listed are provided for clarity only and are not meant to be limiting in any way. Thus, for example, the current disclosure is not limited to IGF-1 for “growth factor”, but rather encompasses and includes all growth factors known in the art.

In some embodiments, Z₁ comprises an amino acid sequence having at least 70% identity to an amino acid sequence selected from the group consisting of SEQ ID NO: 28, 29, 30, 31, 32, 33, 34, 35, 36, 37, 38, 39, 40, 41, 42, 43, 44, or 72. In some embodiments, Z₁ comprises an amino acid sequence having least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to an amino acid sequence selected from the group consisting of SEQ ID NO: 28, 29, 30, 31, 32, 33, 34, 35, 36, 37, 38, 39, 40, 41, 42, 43, 44, or 72. In some embodiments, Z₁ comprises an amino acid sequence selected from the group consisting of SEQ ID NO: 28, 29, 30, 31, 32, 33, 34, 35, 36, 37, 38, 39, 40, 41, 42, 43, 44, or 72.

In some embodiments, a method of producing a payload protein from Escherichia bacteria is provided. In some embodiments, the method comprises providing a nucleic acid encoding a recombinant polypeptide comprising a payload protein and a synthetic pre-protein signal peptide; transfecting the Escherichia bacteria with the nucleic acid, thereby generating an engineered bacteria, culturing the engineered bacteria under effective conditions to grow the bacteria; and inducing secretion an increased amount of payload protein to the culture media, to the periplasm, or a combination thereof, when compared to the amount of payload protein secreted by Escherichia bacteria using a recombinant fusion protein comprising the payload protein and a known signal peptide. In some embodiments, inducing secretion of the payload protein comprises culturing the bacteria under conditions sufficient to express the payload protein, wherein the presence of a pre-protein signal peptide induces secretion of the payload protein to the culture media, to the bacteria cell periplasm, or a combination thereof. In some embodiments, the presence of the pre-protein signal peptide increases secretion to the culture media. In some embodiments, the presence of the pre-protein signal peptide increases secretion to the periplasm. In some embodiments, the known signal peptide may be any known signal peptide. In some embodiments, the known signal peptide is derived from amylase proteins (e.g., SEQ ID NO: 13 or SEQ ID NO: 14). In some embodiments, the known signal peptide comprises an amino acid sequence of SEQ ID NO: 15. In some embodiments, the known signal peptide comprises an amino acid sequence of SEQ ID NO: 66. In some embodiments, the known signal peptide comprises an amino acid sequence of SEQ ID NO: 68. In some embodiments, the known signal peptide comprises an amino acid sequence of SEQ ID NO: 70. In some embodiments, the bacteria is a bacteria as provided for herein. In some embodiments, the bacteria is genetically modified as provided for herein. In some embodiments, the recombinant polypeptide comprises a formula X₁—Z₁, wherein X₁ is a pre-protein signal peptide as provided for herein, and Z₁ is a payload protein. In some embodiments, X₁ comprises an amino acid sequence represented by Formula I or SEQ ID NO: 1, SEQ ID NO: 2, SEQ ID NO: 3, or SEQ ID NO: 4. In some embodiments, X₁ comprises an amino acid sequence of represented by Formula I. In some embodiments, X₁ comprises an amino acid sequence of SEQ ID NO: 1. In some embodiments, X₁ comprises an amino acid sequence of SEQ ID NO: 2. In some embodiments, X₁ comprises an amino acid sequence of SEQ ID NO: 3. In some embodiments, X₁ comprises an amino acid sequence of SEQ ID NO: 4. In some embodiments, X₁ is fused directly or indirectly to Z₁. In some embodiments, X₁ is fused directly to Z₁. In some embodiments, X₁ is fused indirectly to Z₁ via, for example, a linker peptide as provided for herein. In some embodiments, any Escherichia bacteria may be used. In some embodiments, the Escherichia is an Escherichia as provided for herein. In some embodiments, the Escherichia is selected from the group including, but not limited to, E. albertii, E. fergusonii, E. hermannii, E. marmotae, and E. coli. In some embodiments, the Escherichia is E. coli.

In some embodiments, engineered Escherichia bacteria genetically modified with a nucleic acid are provided. In some embodiments, the nucleic acid encodes for the expression of a recombinant polypeptide comprising a synthetic pre-protein signal peptide fused directly or indirectly to a payload protein. In some embodiments, the nucleic acid encodes for the expression of a recombinant polypeptide comprising a formula of X₁—Z₁, wherein X₁ is a pre-protein signal peptide as provided for herein, and Z₁ is a payload protein. In some embodiments, the pre-protein signal peptide is fused directly to the payload protein. In some embodiments, the pre-protein signal peptide is fused indirectly to the payload protein via, for example, a linker peptide as provided for herein. In some embodiments, the nucleic acid encodes for the expression of a recombinant polypeptide comprising a formula of X₁—Z₁, wherein X₁ comprises an amino acid sequence selected from the group consisting of Formula I, SEQ ID NO: 1, SEQ ID NO: 2, SEQ ID NO: 3, and SEQ ID NO: 4. In some embodiments, X₁ comprises an amino acid sequence of represented by Formula I. In some embodiments, X₁ comprises an amino acid sequence of SEQ ID NO: 1. In some embodiments, X₁ comprises an amino acid sequence of SEQ ID NO: 2. In some embodiments, X₁ comprises an amino acid sequence of SEQ ID NO: 3. In some embodiments, X₁ comprises an amino acid sequence of SEQ ID NO: 4. In some embodiments, any Escherichia bacteria may be used. In some embodiments, the Escherichia is a Escherichia as provided for herein. In some embodiments, the Escherichia is selected from the group including but not limited to, E. albertii, E. fergusonii, E. hermannii, E. marmotae, and E. coli. In some embodiments, the Escherichia is E. coli. In some embodiments, the nucleic acid sequence comprises any nucleic acid sequence encoding for the pre-protein signal peptides as provided for herein. In some embodiments, the nucleic acid encoding the amino acid sequence of SEQ ID NO: 1 is SEQ ID NO: 5. In some embodiments, the nucleic acid encoding the amino acid sequence of SEQ ID NO: 2 is SEQ ID NO: 6. In some embodiments, the nucleic acid encoding the amino acid sequence of SEQ ID NO: 3 is SEQ ID NO: 7. In some embodiments, the nucleic acid encoding the amino acid sequence of SEQ ID NO: 4 is SEQ ID NO: 8.

In some embodiments, the nucleic acid encodes for the expression of a recombinant polypeptide comprising a formula of X₁—Z₁, wherein Z₁ is any peptide or protein. In some embodiments, the nucleic acid encodes for the expression of a recombinant polypeptide comprising a formula of X₁—Z₁, wherein Z₁ is selected from the group including, but not limited to, an antiviral, insulin, an incretin, an enzyme, an enzyme inhibitor, a hormone, a cytokine, an antibody, a single domain antibody fragment, a single chain variable antibody fragment, an antimicrobial peptide, a mucosal protein, pesticide, bactericide herbicide, fungicide, nematicide, miticide, plant growth regulator, plant growth stimulator or fertilizer, a vaccine, a diagnostic protein, a feed conversion enzyme, a flavoring, a nutritional protein, venom peptides, endoglucanase, restriction enzymes, human growth hormone, insulin, Beta-lactamase, luciferase (such as, but not limited to, Renilla luciferase or NanoLuc luciferase), phytase, cellulase, chitinase, phosphatase, catalase, urokinase, tissue plasminogen activator, apolipoprotein, beta-glucosidases, hemicellulases, lignocellulose oxireductases, DNAses NADPH dehydrogenase, alcohol oxidase, pyruvate decarboxylase, formolase, alpha-ketoglutarate dehydrogenase, branched chain alpha-ketoacid decarboxylase, copper radical oxidase, galactose oxidase, glycerol oxidase, amine oxidase, glyoxalase, amino monoaxidase, ethylene glycol oxidase, alditol oxidase, or 2-oxoglutarate dehydrogenase. In some embodiments, the nucleic acid encodes for the expression of a recombinant polypeptide comprising a formula of X₁—Z₁, wherein Z₁ is selected from the group including, but not limited to, an enzyme (e.g., invertase, isomaltase, lactase, lysozyme, An-PEP), a growth factor (e.g., IGF1), insulin, an incretin (e.g., GLP-1, GLP-2, leptin, apelin, ghrelin, PYY, nesfatin), a cytokine, an antibody, an antimicrobial peptide), a mucosal protein (e.g., trefoil factor, Reg3 protein, superoxide dismutase), an agricultural product (e.g., pesticide, bactericide herbicide, fungicide, nematicide, miticide, plant growth regulator, plant growth stimulator, or fertilizer), a vaccine, a diagnostic protein, a feed conversion enzyme, a flavoring, or a nutritional protein. In some embodiments, Z₁ is selected from the group including, but not limited to, venom peptides, endoglucanase, restriction enzymes, human growth hormone, insulin, Beta-lactamase, luciferase (such as, but not limited to, Renilla luciferase or NanoLuc luciferase), phytase, cellulase, chitinase, phosphatase, catalase, urokinase, tissue plasminogen activator, apolipoprotein, NADPH dehydrogenase, alcohol oxidase, pyruvate decarboxylase, formolase, alpha-ketoglutarate dehydrogenase, branched chain alpha-ketoacid decarboxylase, copper radical oxidase, galactose oxidase, glycerol oxidase, amine oxidase, glyoxalase, amino monoaxidase, ethylene glycol oxidase, alditol oxidase, or 2-oxoglutarate dehydrogenase. In some embodiments, Z₁ is selected from the group including, but not limited to, amylases, alpha amylases, xylanases (e.g. endo-1,4-beta-xylanase), lichenases (e.g. beta glucanase), lipases (e.g. Candida antartica lipase B, Candida rugose lipase, LipA), pectinases (e.g. pectate trisaccharide lyase), and cellulases (e.g. endoglucanase A). The examples listed are provided for clarity only and are not meant to be limiting in any way. Thus, for example, the current disclosure is not limited to IGF-1 for “growth factor”, but rather encompasses and includes all growth factors known in the art.

In some embodiments, the nucleic acid encodes for the expression of a recombinant polypeptide comprising a formula of X₁—Z₁, wherein Z₁ comprises an amino acid sequence having at least 70% identity to an amino acid sequence selected from the group consisting of SEQ ID NO: 28, 29, 30, 31, 32, 33, 34, 35, 36, 37, 38, 39, 40, 41, 42, 43, 44, or 72. In some embodiments, the nucleic acid encodes for the expression of a recombinant polypeptide comprising a formula of X₁—Z₁, wherein Z₁ comprises an amino acid sequence having least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to an amino acid sequence selected from the group consisting of SEQ ID NO: 28, 29, 30, 31, 32, 33, 34, 35, 36, 37, 38, 39, 40, 41, 42, 43, 44, or 72. In some embodiments, the nucleic acid encodes for the expression of a recombinant polypeptide comprising a formula of X₁—Z₁, wherein Z₁ comprises an amino acid sequence selected from the group consisting of SEQ ID NO: 28, 29, 30, 31, 32, 33, 34, 35, 36, 37, 38, 39, 40, 41, 42, 43, 44, or 72.

### Methods of Generating Engineered Bacteria

Provided herein are synthetic signal peptides that may be used to genetically modify Escherichia bacteria to increase secretion of any payload protein or peptide. A synthetic signal sequence comprises a pre-protein signal peptide (e.g., comprising an amino acid sequence of Formula I, SEQ ID NO: 1, 2, 3, or 4) fused directly or indirectly to a payload protein. Indirect fusion can be via, for example, a linker peptide as provided for herein.

Accordingly, in some embodiments, a method of generating an engineered bacterium that expresses a recombinant polypeptide comprising a synthetic signal peptide and a payload protein is provided. In some embodiments, the method comprises providing a bacterium and contacting the bacteria with a nucleic acid encoding the recombinant polypeptide comprising a synthetic pre-protein signal peptide and a payload protein under conditions suitable to genetically modify the bacterium to induce expression of the recombinant polypeptide, thereby creating an engineered bacterium. In some embodiments, the recombinant polypeptide is as provided for herein. In some embodiments, the pre-protein signal peptide is as provided for herein. In some embodiments, the nucleic acid is as provided for herein.

In some embodiments, the bacteria may be any species of bacteria within the Escherichia genus. In some embodiments, the Escherichia bacteria is as provided for herein. In some embodiments, the Escherichia bacteria is selected from the group including, but not limited to E. albertii, E. fergusonii, E. hermannii, E. marmotae, and E. coli. In some embodiments, the Escherichia is E. coli. In some embodiments, any strain within the species may be used. For example, suitable strains within the E. coli species include, but are not limited to MG1655, NEB Turbo, DH10B, NEB Stable, DH5α, Mach1, BW25113, DB3.1, OmniMAX2, XL1-Blue, NEB dam⁻/dcm⁻, ET12567, EC100D, BW25141, BW2474, BW29655, Marionette-Clo, Marionette-Pro, Marionette-Wild, BL21 (DE3), Rosetta™ (DE3)pLysS, BLIM, BioDesignER (RE1000), Nissle 1917, DH1, JM109, BLR(DE3), BLR(DE3) pRIL, DP10, RU1012, JTK165JJ, BW27783, DGF-298, K-12 strain 58, K-12 strain 679, K12-strain WG1, K-12 derivative strains 5K, 58, 58-161, AN284, AB311, AG1, C600, DP50, EMG2, EPI100-T1R, H1443, HB101, Hfr3000, Hfr 3000 X74, JM109, TG1, TOP10, W1485, W208, W3110, W945, WA704, WG1, JC9387, JM83, JM101, KP7600, LE392, M15, MB408, Novablue, P678, PA 309, REG-12, S17-1, SCS-110, SM10, STBL2, STBL3, TB1, SURE, XL10-Glod, XLOLR, T10, and YN2980, and non-K12 strains B, B-3, B/R, BL23, C, C41, C43, FDA strain Seattle 1946, K5808, Nissle 1917, Rosetta, REG-811, W, and 25922. In some embodiments, inducing expression of the recombinant fusion protein may be carried out via any expression system known to those skilled in the art. For example, in any aspect, a method of genetically modifying a bacterium to generate an engineered bacterium may comprise preparing a vector containing a nucleic acid (e.g., RNA, DNA) encoding the recombinant fusion protein, transporting the vector to the host bacteria (“genetically modifying”), and culturing the bacteria under effective conditions to express the recombinant fusion protein. As used herein, the term “vector” refers to a nucleotide molecule capable of transporting other nucleotides to which it has been linked. One exemplary type of vector is a “plasmid”, which represents a circular double stranded DNA loop into which additional DNA sections can be ligated. Another type of vector is a viral vector; wherein additional DNA sections can be ligated with the viral genome. Methods of introducing a DNA into bacteria are known to those skilled in the art and may include a transformation method, a transfection method, an electroporation method, a nuclear injection method, or a carrier such as a liposome, micelle, skin cell, or a fusion method using protoplasts. A recombinant nucleic acid encoding the recombinant fusion protein may be obtained from any source using conventional techniques known to those skilled in the art, including isolation from genomic or cDNA libraries, amplification by PCR, or chemical synthesis.

In some embodiments, the engineered bacteria may be cultured for a period of time in an environment effective to maintain the health of the bacteria, thereby generating a desired amount of recombinant fusion protein comprising the synthetic signal peptide and payload protein. The culturing of bacteria is common practice and well known in the art. In general, bacteria can be grown in nutrient-rich broth, which may comprise amino acids and nitrogen. Engineered bacteria may be grown for any amount of time necessary to generate the desired amount of recombinant fusion protein comprising the signal peptide and payload protein. For example, the bacteria may be grown for about 0.5 hours to about 168 hours or longer. In some embodiments, bacteria may be grown for 0.5 h, 1 h, 2 h, 3 h, 4 h, 5 h, 6 h, 12 h, 18 h, 24 h, 30) h. 36 h, 42 h, 48 h, 72 h, 96 h, 120 h, 144 h, or 168 hours, or longer. In some embodiments, bacteria may be grown for any time period within any of the recited time periods or longer. Further, the bacteria may be grown in a continuous culture system, whereby a portion of a bacteria culture is seeded into fresh growth broth and the culture is continued. As such, in some embodiments, the bacteria may be grown for at least 0.5 hours. One of skill in the art will recognize that time of growth is a temperature dependent variable, as different temperatures produce different growth rates of bacteria, and as such growth of the bacteria for any time period is within the scope of the present application. Accordingly, engineered bacteria may be grown at room temperature or, more effectively, at a temperature of about 40° C. to 140° C. though any particular species and/or strain will have an optimal temperature range which will be known to one of ordinary skill in the art. Temperature may be used to control the growth of the bacteria and to control the production of the desired fusion protein. Thus, in some embodiments, the bacteria may be cultured at a temperature of about 4° C. to about 140° C. The temperature range used in any of the embodiments herein can be any temperature range within the recited temperature range. Thus, in some embodiments, the bacteria may be cultured at a temperature of about 4° C. to about 140° C., from about 4° C. to about 80° C., from about 4° C. to about 40° C., from about 16° C. to about 40° C., from about 16° C. to about 60° C., from about 22° C. to about 37° C., from about 22° C. to about 45° C., from about 22° C. to about 140° C., and so on. Likewise, the recited temperature ranges include each and every individual temperature within said range. Thus, in some embodiments, the bacteria may be cultured at 4° C. In some embodiments, the bacteria may be cultured at 16° C. In some embodiments, the bacteria may be cultured at 22° C. In some embodiments, the bacteria may be cultured at 25° C. In some embodiments, the bacteria may be cultured at 30° C. In some embodiments, the bacteria may be cultured at 37° C. In some embodiments, the bacteria may be cultured at 4° C., 5° C., 10° C., 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 50° C., 55° C., 60° C., 65° C., 70° C., 75° C., 80° C., 85° C., 90° C., 95° C., 100° C., 105° C., 110° C., 115° C., 120° C., 125° C., 130° C., 135° C., 140° C., or any temperature in between the recited temperatures. Further, those skilled in the art will recognize that further modifications to the growth conditions may be necessary depending on the strain of bacteria utilized and the fusion protein being produced. Such modifications are within the scope of the present application. In any case, secretion of a payload protein by the host bacteria will result in its accumulation in the surrounding culture medium, where it may then be collected, isolated, and/or quantified. Through various intracellular mechanisms, the payload protein will be extracellularly secreted with or without some or all of the synthetic pre-protein signal peptide to which it was fused.

In some embodiments, the engineered bacteria may be grown in any volume of culture media. One of skill in the art will recognize that the volume of culture media necessary for bacteria growth will depend on the amount of payload protein desired to be produced. Accordingly, in some embodiments, the bacteria are cultured in a volume of about 0.005 L to about 1,000,000 L or more. In some embodiments, the bacteria are cultured in a volume of at least 0.005 L. In some embodiments, the bacteria are cultured in a volume of about 0.005 L, 0.05 L, 0.5 L, 1 L, 2 L, 3 L, 4 L, 5 L, 10 L, 20 L, 30 L, 40 L, 50 L, 100 L, 1,000 L, 10,000 L, 100,000 L, or 1,000,000 L or greater. In some embodiments, the bacteria may be cultured at any volume in between any of the recited volumes or greater. Further, the bacteria may be grown in a continuous culture system, whereby a portion of a bacteria culture is seeded into fresh growth broth and the culture is continued. It is to be understood that the volumes recited are in not to be construed as limiting in any way, and that the bacteria may be grown in any volume that is appropriate for payload protein production.

In some embodiments, the fusion protein comprising the signal peptide (X₁) and the payload protein (Z₁) are exported to the periplasm for further processing. In some embodiments, the presence of the signal peptide is sufficient to export the fusion protein to the periplasm. In some embodiments, increased export to the periplasm can be achieved by providing the engineered bacteria with nucleic acid constructs to co-produce cytoplasmic chaperone proteins. In some embodiments, the bacteria are transformed or transfected with the nucleic acid constructs encoding the cytoplasmic chaperone proteins concurrently with the nucleic acid constructs encoding the fusion protein. In some embodiments, the bacteria stably produce the cytoplasmic chaperone proteins. Cytoplasmic chaperone proteins are known in the art and any such chaperone protein is within the scope of the present disclosure Non-limiting examples of cytoplasmic chaperone proteins that may be utilized include GroEL and DnaK.

In some embodiments, increased export to the periplasm can be achieved by providing the bacteria with nucleic acid constructs to co-produce the Sec-translocon core components SecY and SecE. In some embodiments, the bacteria are transformed or transfected with the nucleic acid constructs encoding SecY and SecE concurrently with the nucleic acid constructs encoding the fusion protein. In some embodiments, the bacteria stably produce SecY and SecE. In some embodiments, the Sec-translocon core component is selected from SecY, SecE, or a combination thereof. In some embodiments, increased export to the periplasm can be achieved by providing the bacteria with nucleic acid constructs to co-produce one or more proteins capable of facilitating protein secretion or translocation, such as, but not limited to, SecY, SecE, SecG, SecYEG, SecA, SecB, FtsY, Lep, or any combination thereof.

In some embodiments, the yield of the payload protein (Z₁) in the periplasm can be increased by co-producing signal peptidase I (LepB). Without wishing to be bound by theory, LepB can process the fusion protein by cleaving off the signal peptide upon protein translocation. Such processing can increase periplasmic protein production yields.

In some embodiments, periplasmic protein production yields can be increased via providing the bacteria with nucleic acid constructs to co-produce periplasmic folding modulators. Such folding modulators may be used, for example, to enhance the production of disulfide containing payload proteins in the periplasm. In some embodiments, the bacteria are transformed or transfected with the nucleic acid constructs encoding the periplasmic folding modulators concurrently with the nucleic acid constructs encoding the fusion protein. In some embodiments, the bacteria stably produce the periplasmic folding modulators. Periplasmic folding modulators are known in the art, and any such modulator is within the scope of the present disclosure. Non-limiting examples of periplasmic folding modulators include DsbA, DsbA, DsbC, DsbD, FkpA, SurA, Skp, PPIA, PPiD, and combinations thereof.

In some embodiments, the payload protein (Z₁) that may be produced by the engineered bacteria can be any protein. In some embodiments, the payload proteins that may be produced by the engineered bacteria disclosed herein include, but are not limited to, maltose binding protein (MBP), trefoil factor, mucin. DNase, clotting or blood volumizing factors, insulin and insulin analogs, an incretin (e.g., GLP-1, GLP-2, leptin, apelin, ghrelin, PYY, nesfatin), EGFP, PDGF, HB-EGF, α1-antitrypsin, serum albumin, collagen, pepsinogen, tumor necrosis factor, streptokinase, glucagon, lepirudin, desirudin, hirudin, encallantide, IFN-α 2b, antigens, antibodies, and antibody fragments (e.g., anti-TNFα Ab, anti-IL-6R Ab, anti-RSV ab, tetanus toxin fragment C, An-PEP, HIV-1 gp120 (intracellular), HIV-1 gp120 (secreted), Bm86 tick gut glytoprotein, murine single-chain antibody, anti-TNF Ab, cancer antibodies, sHBsAg, antigen binding fragment/s, single-chain variable fragment (scFv), single-domain antibodies, camelid nanobodies, Shark vNAR, enzymes (e.g., lysozyme, invertase, galactanase, isomaltase, lactase, chitiniase, xylanase, catalase, D-alanine carboxypeptidase, α-amylase, aspartic proteinase II, galactosidase, horseradish peroxidase, rasburicase, ocriplasmin, pancrelipase, alcohol dehydrogenase (I and II), phosphoglyserate kinase. GADPH, acid phosphatase, restriction enzymes, endoglucanases, beta-glucosidases, cellulases, hemicellulases, lignocellulose oxireductases), enzyme inhibitors (e.g., Kunitz, protease inhibitor, tick anticoagulant protein, ghilanten, tPA Kringle type-2 domain), hormones (e.g., HGH, follicle stimulating hormone, human parathyroid hormone), vaccines (e.g., hepatitis vaccine (I), HPV vaccine), food processing products (e.g., brazzein, chymocin, beta-galactosidase), cytokines, amylases, alpha amylases, xylanases (e.g. endo-1,4-beta-xylanase), lichenases (e.g. beta glucanase), lipases (e.g. Candida antartica lipase B, Candida rugose lipase, LipA), pectinases (e.g. pectate trisaccharide lyase), and cellulases (e.g. endoglucanase A). The examples listed are provided for clarity only and are not meant to be limiting in any way. Thus, for example, the current disclosure is not limited to hepatitis vaccine (I) or HPV vaccine for “vaccines”, but rather encompasses and includes all applicable vaccines known in the art.

In some embodiments, secretion of a payload protein by a bacterium may be increased by genetically modifying the bacteria to express the payload protein as part of a recombinant polypeptide comprising a synthetic signal peptide as disclosed herein. Accordingly, in some embodiments, an engineered bacterium may secrete about 10% to about 200% more of a payload protein than a bacterium expressing a native signal peptide. For example, an engineered bacterium may express about 10% to about 50% more, about 20% to about 70% more, about 30% to about 90% more, or about 50% to about 200% more of a payload protein. It is to be understood that any individual percentage of increased payload protein secretion is encompassed within the embodiments described herein. Accordingly, in some embodiments, the bacteria may secrete about 10% more of a payload protein. In some embodiments, the bacteria may secrete about 20% more of a payload protein. In some embodiments, the bacteria may secrete about 10%, about 20%, about 30%, about 40%, about 50%, about 60%, about 70%, about 80%, about 90%, about 100%, about 110%, about 120%, about 130%, about 140%, about 150%, about 160%, about 170%, about 180%, about 190%, or about 200% more of a payload protein, or any percentage falling within any of the recited percentages. Those of skill in the art would recognize that any change in growth condition during routine optimization for expression of a particular polypeptide of interest may also affect the amount of pay load protein secreted by the engineered bacteria. Accordingly, in some embodiment, an engineered bacterium may secrete at least 10% more of a payload protein. Accordingly, in some embodiments, an engineered bacterium may secrete about 10% more, about 100%, about 500% more, about 1000% more, or about 10,000% more of a payload protein compared to a bacteria expressing a native signal peptide. Those of skill in the art will readily appreciate and understand that additional components or conditions may be modified to increase the expression and secretion of the payload protein. For example, one might introduce cytoplasmic chaperone proteins, Sec-translocon core components, periplasmic folding modulators, or any combination thereof, as provided for herein. Such modifications, as provided for or otherwise, are within the scope of the present disclosure. In some embodiments, secretion is measured by any method known in the art, for example, by measuring the concentration of the payload protein in the culture media in which the bacteria was grown. In some embodiments, the secretion is measured by isolating the payload protein from the periplasm of the bacteria and then measuring the concentration of the isolated payload protein. The concentration may be normalized to optical density to account for variations in growth of the bacteria. In some embodiments, secretion is measured by any method known to those skilled in the art for measuring payload protein concentration.

In some embodiments, the payload protein may be isolated from the culture medium in which the engineered bacteria is grown using any methods known to those skilled in the art, such as precipitation from the medium, immunoaffinity chromatography, receptor affinity chromatography, or hydrophobic interaction chromatography. In some embodiments, the payload protein may be isolated by conventional chromatographic methods such as affinity chromatography, size-exclusion filtration, cation or anion exchange chromatography, high pressure liquid chromatography (HPLC), reverse phase HPLC, and the like.

In some embodiments, the pay load protein is isolated from the periplasm of the bacteria. Isolation of proteins from periplasm is known in the art, and any such method is within the scope of the present disclosure. Non-limiting examples of periplasm isolation methods known in the art include a cold osmotic shock method, a chloroform extraction procedure, rapid freeze thaw methods, slow freeze thaw methods, and prolonged heating of the isolated cell pellet (See Lall, S. D., et al, Comparison of four methods for extracting periplasmic proteins, Journal of Microbiological methods, 1989, Vol. 9, Issue 3. pg 195-199, and U.S. Pat. No. 9,725,516; both of which are hereby incorporated by reference in their entirety). These non-limiting methods of periplasmic protein extraction are provided for illustrative purposes only and are not intended to be limiting in any way. Any method of periplasmic protein isolation may be utilized for the embodiments of the present disclosure.

In some embodiments, the payload protein is isolated from both the periplasm and from the culture medium in which the engineered bacteria is grown. Methods for isolation of proteins from periplasm and culture medium are known in the art, and any such methods are within the scope of the present disclosure. Further, the combination of any two methods to allow for the isolation of proteins from periplasm and culture medium from a single batch are also within the scope of the present disclosure.

In some embodiments, the recombinant polypeptide may be designed to comprise a specific affinity peptide, tag, label, or chelate residue that is recognized by a specific binding partner or agent which may aid in isolation. In some embodiments, recombinant polypeptide variants comprising the additional tag, label, or residue may then be cleaved to obtain the payload protein.

### Methods of Using Synthetic Pre-Protein Signal Peptides

In some embodiments, the various signal peptides disclosed herein may be utilized in bacteria to produce and secrete a payload protein. In some embodiments, the engineered bacteria of the present disclosure may be utilized to produce and secrete a payload protein. In some embodiments, engineered bacteria may be used to produce an industrial commodity protein. In some embodiments, the industrial commodity protein is any protein that may be of industrial interest. In some embodiments, the industrial commodity protein is any protein. In some embodiments, the industrial commodity protein is a payload protein as provided for herein. In some embodiments, the industrial commodity protein is selected from the group including, but not limited to, maltose binding protein (MBP), trefoil factor, mucin, DNase, clotting or blood volumizing factors, insulin and insulin analogs, an incretin (e.g., GLP-1, GLP-2, leptin, apelin, ghrelin, PYY, nesfatin), EGFP, PDGF, HB-EGF, α1-antitrypsin, serum albumin, collagen, pepsinogen, tumor necrosis factor, streptokinase, glucagon, lepirudin, desirudin, hirudin, encallantide, IFN-α 2b, antigens, antibodies, and antibody fragments (e.g., anti-TNFα Ab, anti-IL-6R Ab, anti-RSV ab, tetanus toxin fragment C, An-PEP, HIV-1 gp120 (intracellular), HIV-1 gp120 (secreted), Bm86 tick gut glytoprotein, murine single-chain antibody, anti-TNF Ab, cancer antibodies, sHBsAg, antigen binding fragment/s, single-chain variable fragment (scFv), single-domain antibodies, camelid nanobodies, Shark vNAR, enzymes (e.g., lysozyme, invertase, galactanase, isomaltase, lactase, chitiniase, xylanase, catalase, D-alanine carboxypeptidase, α-amylase, aspartic proteinase II, galactosidase, horseradish peroxidase, rasburicase, ocriplasmin, pancrelipase, alcohol dehydrogenase (I and II), phosphoglyserate kinase, GADPH, acid phosphatase), enzyme inhibitors (e.g., Kunitz protease inhibitor, tick anticoagulant protein, ghilanten, tPA Kringle type-2 domain), hormones (e.g., HGH, follicle stimulating hormone, human parathyroid hormone), vaccines (e.g., hepatitis vaccine (I), HPV vaccine), food processing products (e.g., brazzein, chymocin, beta-galactosidase), and cytokines. In some embodiments, the industrial commodity protein is selected from the group including, but not limited to, amylases, alpha amylases, xylanases (e.g. endo-1,4-beta-xylanase), lichenases (e.g. beta glucanase), lipases (e.g. Candida antartica lipase B, Candida rugose lipase, LipA), pectinases (e.g. pectate trisaccharide lyase), and cellulases (e.g. endoglucanase A). It is to be understood that the examples listed are provided for clarity only and are not meant to be limiting in any way. Thus, for example, the current disclosure is not limited to hepatitis vaccine (I) or HPV vaccine for “vaccines”, but rather encompasses and includes all applicable vaccines known in the art.

In some embodiments, the various signal peptides disclosed herein may be utilized in bacteria to deliver any payload protein to any environment. In some embodiments, engineered bacteria genetically modified to express a recombinant polypeptide comprising a pre-protein signal peptide as disclosed herein may be used to deliver one or more of a therapeutic protein, diagnostic protein, or protein-based vaccine to a subject in need thereof. In some embodiments, the engineered bacteria utilizing a signal peptide as disclosed herein may be used to deliver a payload protein to a specific organ or location within the subject. In some embodiments, delivery may be to a subject's GI tract, skin, reproductive tract, or the like. In some embodiments, the subject may be an animal, such as a companion animal (e.g., dog, cat, rodent, or the like). In some embodiments, the subject may be a livestock animal (e.g., cattle, sheep, horse, pig, goat, or the like). In some embodiments, the subject is a human.

In some embodiments, engineered bacteria may be used to deliver one or more of a protein-based herbicide, fungicide, bactericide, insecticide, nematicide, miticide, plant growth regulator, plant growth stimulant, or fertilizer in an agricultural environment, such as to crops or plants (such as seeds, roots, corn, tubers, bulbs, slip, rhizome, grass, or vines) or to a plant growth environment (such as topsoil, top dressing, compost, manure, water table, or hydroponic tank).

In some embodiments, engineered bacteria may be incorporated into a food product, such as, but not limited to, bread, dairy, or fermented beverage, to deliver a therapeutic protein, diagnostic protein, protein-based vaccine, an anti-spoilage agent (e.g., bactericide or fungicide), protein-based flavoring agent, protein supplement, or an allergen degrader (e.g., gluten enzyme).

### Methods of Producing Industrial Commodity Proteins

An engineered bacteria may be used to produce industrial commodity proteins. As used herein, “industrial commodity protein” is understood to be any protein that has or may have industrial or commercial use. Accordingly, in some embodiments, a method for producing an industrial commodity protein is provided, the method comprising transfecting a bacterium with a nucleic acid molecule encoding for a recombinant polypeptide comprising a formula of X₁—Z₁, wherein X₁ is a pre-protein signal peptide and Z₁ is a payload protein comprising an industrial commodity protein, thereby producing a bacterium comprising the nucleic acid molecule; culturing the bacteria comprising the nucleic acid molecule under conditions sufficient to grow the bacteria; and inducing secretion of the payload protein by the bacteria. It is to be understood that “secretion” is meant to encompass both secretion to the cell culture media, and secretion to an extra cytoplasmic space, such as the periplasm. In some embodiments, inducing secretion of the payload protein comprises culturing the bacteria under conditions sufficient to express the payload protein, wherein the presence of the pre-protein signal peptide induces secretion of the payload protein to the culture media, to the bacteria cell periplasm, or a combination thereof. In some embodiments, the payload protein is secreted to the culture media. In some embodiments, the payload protein is secreted to the periplasm. In some embodiments, inducing secretion of the payload protein comprises culturing the bacteria under conditions sufficient to express the polypeptide, wherein the presence of the pre-protein signal peptide induces secretion of the payload protein. In some embodiments, culturing the bacteria comprises incubating the bacteria in culture media. In some embodiments, incubating the bacteria in performed for a certain time and temperature as provided for herein. In some embodiments, the method further comprises recovering or purifying the payload protein from the culture media, the cell periplasm, or a combination thereof. In some embodiments, the method further comprises recovering or purifying the payload protein from the periplasm. In some embodiments, the method further comprises recovering or purifying the payload protein from the culture media. In some embodiments, recovering or purifying the payload protein from the periplasm is as provided for herein. In some embodiments, recovering or purifying the payload protein from the culture media is as provided for herein. In some embodiments, the engineered bacteria may be any Escherichia bacteria as provided for herein. In some embodiments, the Escherichia bacteria is selected from the group including, but not limited to, E. albertii, E. fergusonii, E. hermannii, E. marmotae, and E. coli. In some embodiments, the Escherichia is E. coli.

In some embodiments, the pre-protein signal peptide X₁ comprises an amino acid sequence represented by Formula I, SEQ ID NO: 1, 2, 3, or 4. In some embodiments, the pre-protein signal peptide X₁ comprises an amino acid sequence represented by Formula I. In some embodiments, the pre-protein signal peptide X₁ comprises an amino acid sequence of SEQ ID NO: 1. In some embodiments, the pre-protein signal peptide X₁ comprises an amino acid sequence of SEQ ID NO: 2. In some embodiments, the pre-protein signal peptide X₁ comprises an amino acid sequence of SEQ ID NO: 3. In some embodiments, the pre-protein signal peptide X₁ comprises an amino acid sequence of SEQ ID NO: 4.

In some embodiments, the industrial commodity protein is any protein. In some embodiments, the industrial commodity protein is a therapeutic payload protein such as, but not limited to, those provided for herein. In some embodiments, the industrial commodity protein is an agricultural payload protein such as, but not limited to, those provided for herein. In some embodiments, the industrial payload protein is selected from the group comprising amylases, alpha-amylases, xylanases (e.g. endo-1,4-beta-xylanase), lichenases (e.g. beta glucanase), lipases (e.g. Candida antartica lipase B, Candida rugose lipase. LipA), pectinases (e.g. pectate trisaccharide lyase), and cellulases (e.g. endoglucanase A).

In some embodiments, the pre-protein signal peptide X₁ and the payload protein comprising an industrial commodity protein Z₁ are fused directly. In some embodiments, X₁ and Z₁ are fused indirectly via, for example, a peptide linker as provided for herein. In some embodiments, the peptide linker is a cleavable linker as provided for herein. In some embodiments, the recombinant polypeptide may be designed to further comprise a specific affinity peptide, tag, label, or chelate residue that is recognized by a specific binding partner or agent which may aid in isolation. In some embodiments, recombinant polypeptide variants comprising the additional tag, label, or residue may then be cleaved to obtain the payload protein.

Alpha-amylase catalyzes the cleavage of α-1,4-glucosidic bonds, releasing glucose from starch and it is widely used in the textile and paper industries. Accordingly, in some embodiments, a method of producing alpha-amylase is provided. In some embodiments, the method comprises transfecting a bacterium with a nucleic acid molecule encoding for a recombinant polypeptide comprising alpha-amylase and a pre-protein signal peptide, thereby producing a bacterium comprising the nucleic acid molecule: culturing the bacteria comprising the nucleic acid molecule under conditions sufficient to grow the bacteria; and inducing secretion of alpha-amylase by the bacteria, thereby producing alpha-amylase. In some embodiments, the alpha-amylase is represented by SEQ ID NO: 39, or a sequence that is substantially similar to SEQ ID NO: 39. In some embodiments, inducing secretion of alpha-amylase comprises culturing the bacteria under conditions sufficient to express the alpha-amylase, wherein the presence of the pre-protein signal peptide induces secretion of alpha-amylase. In some embodiments, the alpha-amylase is secreted to the culture media, the periplasm, or a combination thereof. In some embodiments the alpha-amylase is secreted to the periplasm. In some embodiments, the alpha-amylase is secreted to the culture media. In some embodiments, culturing the bacteria comprises incubating the bacteria in culture media. In some embodiments, incubating the bacteria is performed for a certain time and temperature as provided for herein. In some embodiments, the method further comprises recovering or purifying alpha-amylase from the culture media, the cell periplasm, or a combination thereof. In some embodiments, the method further comprises recovering or purifying the alpha-amylase from the cell periplasm. In some embodiments, the method further comprises recovering or purifying the alpha-amylase from the culture media. In some embodiments, recovering or purifying alpha-amylase from the periplasm is as provided for herein. In some embodiments, recovering or purifying alpha-amylase from the culture media is as provided for herein. In some embodiments, the engineered bacteria may be any Escherichia bacteria as provided for herein. In some embodiments, the Escherichia bacteria is selected from the group including, but not limited to, E. albertii, E. fergusonii, E. hermannii, E. marmotae, and E. coli. In some embodiments, the Escherichia is E. coli. In some embodiments, the pre-protein signal peptide comprises an amino acid sequence represented by Formula I, SEQ ID NO: 1, 2, 3, or 4. In some embodiments, the pre-protein signal peptide comprises an amino acid sequence represented by Formula I. In some embodiments, the pre-protein signal peptide comprises an amino acid sequence of SEQ ID NO: 1. In some embodiments, the pre-protein signal peptide comprises an amino acid sequence of SEQ ID NO: 2. In some embodiments, the pre-protein signal peptide comprises an amino acid sequence of SEQ ID NO: 3. In some embodiments, the pre-protein signal peptide comprises an amino acid sequence of SEQ ID NO: 4.

Xylanases are enzymes that catalyze the hydrolysis of β-1,4 glycosidic linkages of xylans, releasing oligosaccharides and disaccharides containing reducing sugars and xylose. They have significant application value in biotechnology and can be used to modify lignocellulosic materials. Xylanases are used in animal feed manufacturing, the paper and textile industries, and biofuel production. Accordingly, in some embodiments, a method of producing xylanases is provided. In some embodiments, the method comprises transfecting a bacterium with a nucleic acid molecule encoding for a recombinant polypeptide comprising a xylanase and a pre-protein signal peptide, thereby producing a bacterium comprising the nucleic acid molecule: culturing the bacteria comprising the nucleic acid molecule under conditions sufficient to grow the bacteria; and inducing secretion of the xylanase by the bacteria, thereby producing a xylanase. In some embodiments, the xylanase can be any xylanase. In some embodiments, the xylanase is Endo-1,4-beta-xylanase. In some embodiments, the xylanase is represented by SEQ ID NO: 40, or a sequence substantially similar to SEQ ID NO: 40. In some embodiments, inducing secretion of the xylanase comprises culturing the bacteria under conditions sufficient to express the polypeptide, wherein the presence of the pre-protein signal peptide induces secretion of the xylanase. In some embodiments, the xylanase is secreted to the culture media, the periplasm, or a combination thereof. In some embodiments the xylanase is secreted to the periplasm. In some embodiments, the xylanase is secreted to the culture media. In some embodiments, culturing the bacteria comprises incubating the bacteria in culture media. In some embodiments, incubating the bacteria in performed for a certain time and temperature as provided for herein. In some embodiments, the method further comprises recovering or purifying the xylanase from the culture media, the cell periplasm, or a combination thereof. In some embodiments, the method further comprises recovering or purifying the xylanase from the cell periplasm. In some embodiments, the method further comprises recovering or purifying the xylanase from the culture media. In some embodiments, recovering or purifying the xylanase from the periplasm is as provided for herein. In some embodiments, recovering or purifying the xylanase from the culture media is as provided for herein. In some embodiments, the engineered bacteria may be any Escherichia bacteria as provided for herein. In some embodiments, the Escherichia bacteria is selected from the group including, but not limited to, E. albertii, E. fergusonii, E. hermannii, E. marmotae, and E. coli. In some embodiments, the Escherichia is E. coli. In some embodiments, the pre-protein signal peptide comprises an amino acid sequence represented by Formula I, SEQ ID NO: 1, 2, 3, or 4. In some embodiments, the pre-protein signal peptide comprises an amino acid sequence represented by Formula I. In some embodiments, the pre-protein signal peptide comprises an amino acid sequence of SEQ ID NO: 1. In some embodiments, the pre-protein signal peptide comprises an amino acid sequence of SEQ ID NO: 2. In some embodiments, the pre-protein signal peptide comprises an amino acid sequence of SEQ ID NO: 3. In some embodiments, the pre-protein signal peptide comprises an amino acid sequence of SEQ ID NO: 4.

Lichenase is a mixed linked β-glucan endo-hydrolase found in both microorganisms and plants, which has become a focus of studies on the feasibility of biofuel production. Accordingly, in some embodiments, a method of producing lichenase is provided. In some embodiments, the method comprises transfecting a bacterium with a nucleic acid molecule encoding for a recombinant polypeptide comprising lichenase and a pre-protein signal peptide, thereby producing a bacterium comprising the nucleic acid molecule; culturing the bacteria comprising the nucleic acid molecule under conditions sufficient to grow the bacteria; and inducing secretion of lichenase by the bacteria, thereby producing lichenase. In some embodiments, the lichenase can be any lichenase. In some embodiments, the lichenase is beta-glucanase. In some embodiments, the lichenase is represented by SEQ ID NO: 41, or a sequence substantially similar to SEQ ID NO: 41. In some embodiments, inducing secretion of lichenase comprises culturing the bacteria under conditions sufficient to express the polypeptide, wherein the presence of the pre-protein signal peptide induces secretion of lichenase. In some embodiments, the lichenase is secreted to the culture media, the periplasm, or a combination thereof. In some embodiments the lichenase is secreted to the periplasm. In some embodiments, the lichenase is secreted to the culture media. In some embodiments, culturing the bacteria comprises incubating the bacteria in culture media. In some embodiments, incubating the bacteria in performed for a certain time and temperature as provided for herein. In some embodiments, the method further comprises recovering or purifying lichenase from the culture media, the cell periplasm, or a combination thereof. In some embodiments, the method further comprises recovering or purifying the lichenase from the cell periplasm. In some embodiments, the method further comprises recovering or purifying the lichenase from the culture media. In some embodiments, recovering or purifying lichenase from the periplasm is as provided for herein. In some embodiments, recovering or purifying lichenase from the culture media is as provided for herein. In some embodiments, the engineered bacteria may be any Escherichia bacteria as provided for herein. In some embodiments, the Escherichia bacteria is selected from the group including, but not limited to, E. albertii, E. fergusonii, E. hermannii, E. marmotae, and E. coli. In some embodiments, the pre-protein signal peptide comprises an amino acid sequence represented by Formula I, SEQ ID NO: 1, 2, 3, or 4. In some embodiments, the pre-protein signal peptide comprises an amino acid sequence represented by Formula I. In some embodiments, the pre-protein signal peptide comprises an amino acid sequence of SEQ ID NO: 1. In some embodiments, the pre-protein signal peptide comprises an amino acid sequence of SEQ ID NO: 2. In some embodiments, the pre-protein signal peptide comprises an amino acid sequence of SEQ ID NO: 3. In some embodiments, the pre-protein signal peptide comprises an amino acid sequence of SEQ ID NO: 4.

Lipases are a family of enzymes that catalyze the hydrolysis of fats. Some lipases display broad substrate scope including esters of cholesterol, phospholipids, and lipid-soluble vitamins. Lipases are used commercially, for example, in laundry detergents with several thousand tons per year being produced for this role. Additionally, lipases have been evaluated for the conversion of triglycerides into biofuels, and for the enantioselective synthesis of fine chemicals. Accordingly, in some embodiments, a method of producing lipases is provided. In some embodiments, the method comprises transfecting a bacterium with a nucleic acid molecule encoding for a recombinant polypeptide comprising a lipase and a pre-protein signal peptide, thereby producing a bacterium comprising the nucleic acid molecule; culturing the bacteria comprising the nucleic acid molecule under conditions sufficient to grow the bacteria; and inducing secretion of the lipase by the bacteria, thereby producing a lipase. In some embodiments, the lipase is any lipase. In some embodiments, the lipase is selected from the group comprising Candida antartica lipase B, Candida rugose lipase, and B, subtilis LipA (Lipase EstA). In some embodiments, the lipase is represented by SEQ ID NO: 42, or a sequence substantially similar to SEQ ID NO: 42. In some embodiments, inducing secretion of the lipase comprises culturing the bacteria under conditions sufficient to express the polypeptide, wherein the presence of the pre-protein signal peptide induces secretion of the lipase. In some embodiments, the lipase is secreted to the culture media, the periplasm, or a combination thereof. In some embodiments the lipase is secreted to the periplasm. In some embodiments, the lipase is secreted to the culture media. In some embodiments, culturing the bacteria comprises incubating the bacteria in culture media. In some embodiments, incubating the bacteria in performed for a certain time and temperature as provided for herein. In some embodiments, the method further comprises recovering or purifying the lipase from the culture media, the cell periplasm, or a combination thereof. In some embodiments, the method further comprises recovering or purifying the lipase from the cell periplasm. In some embodiments, the method further comprises recovering or purifying the lipase from the culture media. In some embodiments, recovering or purifying the lipase from the periplasm is as provided for herein. In some embodiments, recovering or purifying the lipase from the culture media is as provided for herein. In some embodiments, the engineered bacteria may be any Escherichia bacteria as provided for herein. In some embodiments, the Escherichia bacteria is selected from the group including, but not limited to, E. albertii, E. fergusonii, E. hermannii, E. marmotae, and E. coli. In some embodiments, the Escherichia is E. coli. In some embodiments, the pre-protein signal peptide comprises an amino acid sequence represented by Formula I, SEQ ID NO: 1, 2, 3, or 4. In some embodiments, the pre-protein signal peptide comprises an amino acid sequence represented by Formula I. In some embodiments, the pre-protein signal peptide comprises an amino acid sequence of SEQ ID NO: 1. In some embodiments, the pre-protein signal peptide comprises an amino acid sequence of SEQ ID NO: 2. In some embodiments, the pre-protein signal peptide comprises an amino acid sequence of SEQ ID NO: 3. In some embodiments, the pre-protein signal peptide comprises an amino acid sequence of SEQ ID NO: 4.

Pectinases are a group of enzymes that break down pectin through hydrolysis, transelimination, and deesterfication reactions. Pectinases are used in both the fruit juice and wine industries, and are also used for retting in the textile industry. Accordingly, in some embodiments, a method of producing pectinases is provided. In some embodiments, the method comprises transfecting a bacterium with a nucleic acid molecule encoding for a recombinant polypeptide comprising a pectinase and a pre-protein signal peptide, thereby producing a bacterium comprising the nucleic acid molecule; culturing the bacteria comprising the nucleic acid molecule under conditions sufficient to grow the bacteria; and inducing secretion of the pectinase by the bacteria, thereby producing a pectinase. In some embodiments, the pectinase can be any pectinase. In some embodiments, the pectinase is pectate trisaccharide lyases. In some embodiments, the pectinase is represented by SEQ ID NO: 43, or a sequence substantially similar to SEQ ID NO: 43. In some embodiments, inducing secretion of the pectinase comprises culturing the bacteria under conditions sufficient to express the polypeptide, wherein the presence of the pre-protein signal peptide induces secretion of the pectinase. In some embodiments, the pectinase is secreted to the culture media, the periplasm, or a combination thereof. In some embodiments the pectinase is secreted to the periplasm. In some embodiments, the pectinase is secreted to the culture media. In some embodiments, culturing the bacteria comprises incubating the bacteria in culture media. In some embodiments, incubating the bacteria in performed for a certain time and temperature as provided for herein. In some embodiments, the method further comprises recovering or purifying the pectinase from the culture media, the cell periplasm, or a combination thereof. In some embodiments, the method further comprises recovering or purifying the pectinase from the cell periplasm. In some embodiments, the method further comprises recovering or purifying the pectinase from the culture media. In some embodiments, recovering or purifying the pectinase from the periplasm is as provided for herein. In some embodiments, recovering or purifying the pectinase from the culture media is as provided for herein. In some embodiments, the engineered bacteria may be any Escherichia bacteria as provided for herein. In some embodiments, the Escherichia bacteria is selected from the group including, but not limited to, E. albertii, E. fergusonii, E. hermannii, E. marmotae, and E. coli. In some embodiments, the Escherichia is E. coli. In some embodiments, the pre-protein signal peptide comprises an amino acid sequence represented by Formula I, SEQ ID NO: 1, 2, 3, or 4. In some embodiments, the pre-protein signal peptide comprises an amino acid sequence represented by Formula I. In some embodiments, the pre-protein signal peptide comprises an amino acid sequence of SEQ ID NO: 1. In some embodiments, the pre-protein signal peptide comprises an amino acid sequence of SEQ ID NO: 2. In some embodiments, the pre-protein signal peptide comprises an amino acid sequence of SEQ ID NO: 3. In some embodiments, the pre-protein signal peptide comprises an amino acid sequence of SEQ ID NO: 4.

Cellulases are a group of enzymes that catalyze the decomposition of cellulose and of some related polysaccharides. Cellulases have a wide variety of commercial uses including uses in food processing, the textile industry, laundry detergents, the pulp and paper industry, pharmaceutical applications, and the fermentation of biomass into biofuels. Accordingly, in some embodiments, a method of producing cellulases is provided. In some embodiments, the method comprises transfecting a bacterium with a nucleic acid molecule encoding for a recombinant polypeptide comprising a cellulase and a pre-protein signal peptide, thereby producing a bacterium comprising the nucleic acid molecule: culturing the bacteria comprising the nucleic acid molecule under conditions sufficient to grow the bacteria; and inducing secretion of the cellulase by the bacteria, thereby producing a cellulase. In some embodiments, the cellulase can be any cellulase. In some embodiments, the cellulase is endoglucanase A. In some embodiments, the cellulase is represented by SEQ ID NO: 44, or a sequence substantially similar to SEQ ID NO: 44. In some embodiments, inducing secretion of the cellulase comprises culturing the bacteria under conditions sufficient to express the polypeptide, wherein the presence of the pre-protein signal peptide induces secretion of the cellulase. In some embodiments, the cellulase is secreted to the culture media, the periplasm, or a combination thereof. In some embodiments the cellulase is secreted to the periplasm. In some embodiments, the cellulase is secreted to the culture media. In some embodiments, culturing the bacteria comprises incubating the bacteria in culture media. In some embodiments, incubating the bacteria in performed for a certain time and temperature as provided for herein. In some embodiments, the method further comprises recovering or purifying the cellulase from the culture media, the cell periplasm, or a combination thereof. In some embodiments, the method further comprises recovering or purifying the cellulase from the cell periplasm. In some embodiments, the method further comprises recovering or purifying the cellulase from the culture media. In some embodiments, recovering or purifying the cellulase from the periplasm is as provided for herein. In some embodiments, recovering or purifying the cellulase from the culture media is as provided for herein. In some embodiments, the engineered bacteria may be any Escherichia bacteria as provided for herein. In some embodiments, the Escherichia bacteria is selected from the group including, but not limited to, E. albertii, E. fergusonii, E. hermannii, E. marmotae, and E. coli. In some embodiments, the Escherichia is E. coli. In some embodiments, the pre-protein signal peptide comprises an amino acid sequence represented by Formula I, SEQ ID NO: 1, 2, 3, or 4. In some embodiments, the pre-protein signal peptide comprises an amino acid sequence represented by Formula I. In some embodiments, the pre-protein signal peptide comprises an amino acid sequence of SEQ ID NO: 1. In some embodiments, the pre-protein signal peptide comprises an amino acid sequence of SEQ ID NO: 2. In some embodiments, the pre-protein signal peptide comprises an amino acid sequence of SEQ ID NO: 3. In some embodiments, the pre-protein signal peptide comprises an amino acid sequence of SEQ ID NO: 4.

Therapeutic Compositions and Methods of their Use

The synthetic pre-protein signal peptides and methods for their use, as disclosed herein, may be used to facilitate secretion of a therapeutic protein by a bacterium. Accordingly, in some embodiments a composition is provided. In some embodiments, the composition comprises a therapeutically effective amount of a therapeutic payload protein, wherein the therapeutic payload protein is generated by an engineered bacterium genetically modified with a nucleic acid molecule encoding a recombinant fusion protein comprising a synthetic pre-protein signal peptide. In some embodiments, the composition further comprises pharmaceutically acceptable carriers or excipients. In some embodiments, the therapeutic protein may be used to treat a condition, disorder, or disease in a subject. Accordingly, in some embodiments, a method of treating a condition, disorder, or disease in a subject in need thereof is provided. In some embodiments, the method comprises administering a composition comprising a therapeutically effective amount of a protein, wherein the protein is produced in an engineered bacterium genetically modified with a nucleic acid encoding a recombinant polypeptide comprising a synthetic pre-protein signal peptide and the protein. In some embodiments, the pre-protein signal peptide comprises an amino acid sequence represented by Formula I, SEQ ID NO: 1, SEQ ID NO: 2, SEQ ID NO: 3, or SEQ ID NO: 4. In some embodiments, the pre-protein signal peptide comprises an amino acid sequence of represented by Formula I. In some embodiments, the pre-protein signal peptide comprises an amino acid sequence of SEQ ID NO: 1. In some embodiments, the pre-protein signal peptide comprises an amino acid sequence of SEQ ID NO: 2. In some embodiments, the pre-protein signal peptide comprises an amino acid sequence of SEQ ID NO: 3. In some embodiments, the pre-protein signal peptide comprises an amino acid sequence of SEQ ID NO: 4. In some embodiments, administering may be performed via any route, such as oral or topical. In some embodiments, the composition is administered orally. In some embodiments, the composition is administered topically. In some embodiments, the disease or condition may include, but is not limited to, an infection, an autoimmune disease, enzymatic deficiencies, diabetes, metabolic disorders, intestinal bacterial overgrowth, bacterial vaginosis, short bowel syndrome, inflammatory bowel disease, colitis, peptic ulcer, gastritis, polyps, hemorrhoids, cirrhosis, or a cancer. In some embodiments, the composition comprising a therapeutic protein that is produced by any engineered bacteria disclosed herein may be formulated for oral, topical, parenteral, or transdermal administration. These compositions may be in form of pill, tablet, capsule, microcapsule, powder, sachet, dragee, gel, liquid, suspension, solution, food product, cream or granule, and may further comprise one or more pharmaceutically acceptable excipients such as, but not limited to, carriers, solvents, co-solvents, emulsifiers, lubricants, disintegrants, binders, fillers, glidants, rheology agents, solubilizers, antimicrobials, antioxidants, preservatives, colorants, flavor agents, emollients, pH modifiers, and the like.

In some embodiments, food products may include, but are not limited to, a dairy product, a yoghurt, an ice cream, a milk-based drink, a milk-based garnish, a pudding, a milkshake, an ice tea, a fruit juice, a diet drink, a soda, a sports drink, a powdered drink mixture for dietary supplementation, an infant and baby food, a calcium-supplemented orange juice, a sauce or a soup.

In some embodiments, engineered bacteria may be administered to a subject and function as a conduit for in vivo drug delivery to the subject. For example, an orally administered engineered bacteria may continue to produce and secrete a therapeutic payload protein within the subject, therefore providing a therapeutic benefit to the subject. Accordingly, in some embodiments, a composition is provided. In some embodiments, the composition comprises a therapeutically effective amount of engineered bacteria genetically modified with a nucleic acid encoding a recombinant polypeptide comprising a synthetic pre-protein signal peptide and a payload protein. In some embodiments, the synthetic pre-protein signal peptide comprises an amino acid sequence represented by Formula I, SEQ ID NO: 1, SEQ ID NO: 2, SEQ ID NO: 3 or SEQ ID NO: 4. In some embodiments, the synthetic pre-protein signal peptide comprises an amino acid sequence of represented by Formula I. In some embodiments, the synthetic pre-protein signal peptide comprises an amino acid sequence of SEQ ID NO: 1. In some embodiments, the synthetic pre-protein signal peptide comprises an amino acid sequence of SEQ ID NO: 2. In some embodiments, the synthetic pre-protein signal peptide comprises an amino acid sequence of SEQ ID NO: 3. In some embodiments, the synthetic pre-protein signal peptide comprises an amino acid sequence of SEQ ID NO: 4. In some embodiments, the composition further comprises pharmaceutically acceptable carriers or excipients.

In some embodiments, a method of treating a condition, disorder, or disease in a subject in need thereof is provided. In some embodiments, the method comprises administering to the subject a therapeutically effective amount of a therapeutic payload protein, wherein the therapeutic payload protein is generated by an engineered bacterium genetically modified with a nucleic acid molecule encoding a recombinant fusion protein comprising a synthetic pre-protein signal peptide and the protein. In some embodiments, the pre-protein signal peptide comprises an amino acid sequence represented by Formula I, SEQ ID NO: 1, SEQ ID NO: 2, SEQ ID NO: 3, or SEQ ID NO: 4. In some embodiments, the pre-protein signal peptide comprises an amino acid sequence of represented by Formula I. In some embodiments, the pre-protein signal peptide comprises an amino acid sequence of SEQ ID NO: 1. In some embodiments, the pre-protein signal peptide comprises an amino acid sequence of SEQ ID NO: 2. In some embodiments, the pre-protein signal peptide comprises an amino acid sequence of SEQ ID NO: 3. In some embodiments, the pre-protein signal peptide comprises an amino acid sequence of SEQ ID NO: 4. In some embodiments, administering may be performed via any route, such as oral or topical. In some embodiments, the therapeutic payload protein is administered orally. In some embodiments, the therapeutic payload protein is administered topically.

In some embodiments, the method of treating a condition, disorder, or disease in a subject in need thereof comprises administering to the subject a therapeutically effective amount of engineered bacteria genetically modified with a nucleic acid encoding a recombinant polypeptide comprising a synthetic pre-protein signal peptide and a payload protein. In some embodiments, the synthetic pre-protein signal peptide comprises an amino acid sequence represented by Formula I, SEQ ID NO: 1, SEQ ID NO: 2, SEQ ID NO: 3, or SEQ ID NO: 4. In some embodiments, the synthetic pre-protein signal peptide comprises an amino acid sequence of represented by Formula I. In some embodiments, the synthetic pre-protein signal peptide comprises an amino acid sequence of SEQ ID NO: 1. In some embodiments, the synthetic pre-protein signal peptide comprises an amino acid sequence of SEQ ID NO: 2. In some embodiments, the synthetic pre-protein signal peptide comprises an amino acid sequence of SEQ ID NO: 3. In some embodiments, the synthetic pre-protein signal peptide comprises an amino acid sequence of SEQ ID NO: 4. In some embodiments, the method comprises administering a composition comprising the engineered bacteria to a subject in need thereof. In some embodiments, the composition further comprises pharmaceutically acceptable carriers or excipients.

In some embodiments, administering may be performed via any route, such as oral or topical. In some embodiments, the disease or condition may include, but is not limited to, an infection, an autoimmune disease, enzymatic deficiencies, diabetes, obesity, metabolic disorders, intestinal bacterial overgrowth, enteric infection, bacterial vaginosis, short bowel syndrome, inflammatory bowel disease, irritable bowel syndrome, small bowel syndrome, Celiac disease, gluten intolerance, colitis, peptic ulcer, gastritis, polyps, hemorrhoids, cirrhosis, or a cancer. In some embodiments, a composition comprising a therapeutic protein that is produced by any engineered bacteria disclosed herein may be formulated for oral, topical, parenteral, or transdermal administration. These compositions may be in form of pill, tablet, capsule, microcapsule, powder, sachet, dragee, gel, liquid, suspension, solution, food product, cream or granule, and may further comprise one or more pharmaceutically acceptable excipients such as, but not limited to, carriers, solvents, co-solvents, emulsifiers, lubricants, disintegrants, binders, fillers, glidants, rheology agents, solubilizers, antimicrobials, antioxidants, preservatives, colorants, flavor agents, emollients, pH modifiers, and the like. The therapeutically effective amount of engineered bacteria may be measured or specified in colony forming units (CFUs) and may be any amount, such as from about 100 CFUs to 10²⁰ CFUs, about 10³ to 10¹⁵ CFUs, 10⁴ to 10¹⁰ CFUs, or about 10² to about 10⁸ CFUs. In some embodiments, the therapeutically effective amount of engineered bacteria is from about 100 CFUs to about 10²⁰ CFUs. In some embodiments, the therapeutically effective amount of engineered bacteria is from about 10³ to about 10¹⁵ CFUs. In some embodiments, the therapeutically effective amount of engineered bacteria is from about 100 CFUs, about 10³ CFUs, or about 10⁴ CFUs to about 10⁸ CFUs, about 10¹⁰ CFUs, about 10¹⁵ CFUs, or about 10²⁰ CFUs. In some embodiments, the therapeutically effective amount of engineered bacteria is any amount of CFU that falls within any of the above ranges

### Methods of Treating Enzyme Deficiency

An engineered Escherichia bacterium may be used, for example, to treat an enzyme deficiency, such as (but not limited to) lactose intolerance (deficiency of lactase), congenital sucrose-isomaltase deficiency (deficiency of sucrase and/or isomaltase), deficiency of pancrelipase (common in many pancreatic disorders), or Celiac disease/gluten intolerance (deficiency of Aspergillus niger prolyl endoprotease (An-PEP)). In some embodiments, the bacterium is used to produce a therapeutic payload protein useful for treating the enzyme deficiency. In some embodiments, the bacteria producing the therapeutic payload protein is useful for treating the enzyme deficiency.

Accordingly, in some embodiments, a method of treating an enzyme deficiency in a subject in need thereof is provided, the method comprising administering to the subject a therapeutically effective amount a therapeutic pay load protein, wherein the therapeutic payload protein is generated by an engineered bacterium genetically modified with a nucleic acid molecule encoding a recombinant fusion protein comprising a synthetic pre-protein signal peptide and the protein, thereby treating the enzyme deficiency. In some embodiments, the subject is deficient in an enzyme as provided for herein. In some embodiments, the subject is deficient in an enzyme selected from the group comprising lactase, sucrase, isomaltase, An-PEP, or pancrelipase. In some embodiments, the synthetic signal peptide is a pre-protein signal peptide comprising an amino acid sequence represented by Formula I, SEQ ID NO: 1, 2, 3, or 4. In some embodiments, the synthetic pre-protein signal peptide comprises an amino acid sequence of represented by Formula I. In some embodiments, the synthetic pre-protein signal peptide comprises an amino acid sequence of SEQ ID NO: 1. In some embodiments, the synthetic pre-protein signal peptide comprises an amino acid sequence of SEQ ID NO: 2. In some embodiments, the synthetic pre-protein signal peptide comprises an amino acid sequence of SEQ ID NO: 3. In some embodiments, the synthetic pre-protein signal peptide comprises an amino acid sequence of SEQ ID NO: 4. In some embodiments, the engineered bacteria may be any Escherichia bacteria. In some embodiments, the Escherichia bacteria is a Escherichia bacteria as provided for herein. In some embodiments, the Escherichia bacteria is selected from the group including, but not limited to, E. albertii, E. fergusonii, E. hermannii, E. marmotae, and E. coli. In some embodiments, the Escherichia is E. coli. In some embodiments, the therapeutic payload protein or composition comprising the therapeutic payload protein may be administered to the subject by any effective route. In some embodiments, the route of administration is oral.

In some embodiments, the method of treating an enzyme deficiency in a subject in need thereof comprises administering to the subject a therapeutically effective amount of an engineered bacteria genetically modified to express a recombinant polypeptide comprising the enzyme of which the subject is deficient and a synthetic signal peptide, thereby treating the enzyme deficiency. In some embodiments, the subject is deficient in an enzyme as provided for herein. In some embodiments, the subject is deficient in an enzyme selected from the group comprising lactase, sucrase, isomaltase, An-PEP, or pancrelipase. In some embodiments, the synthetic signal peptide is a pre-protein signal peptide comprising an amino acid sequence represented by Formula I, SEQ ID NO: 1, 2, 3, or 4. In some embodiments, the synthetic pre-protein signal peptide comprises an amino acid sequence of represented by Formula I. In some embodiments, the synthetic pre-protein signal peptide comprises an amino acid sequence of SEQ ID NO: 1. In some embodiments, the synthetic pre-protein signal peptide comprises an amino acid sequence of SEQ ID NO: 2. In some embodiments, the synthetic pre-protein signal peptide comprises an amino acid sequence of SEQ ID NO: 3. In some embodiments, the synthetic pre-protein signal peptide comprises an amino acid sequence of SEQ ID NO: 4. In some embodiments, the method comprises administering to the subject in need thereof a composition comprising a therapeutically effective amount of an engineered bacteria genetically modified to express a recombinant polypeptide comprising the enzyme of which the subject is deficient and a synthetic signal peptide comprising a synthetic pre-protein signal peptide. In some embodiments, the engineered bacteria may be any Escherichia bacteria. In some embodiments, the Escherichia bacteria is a Escherichia bacteria as provided for herein. In some embodiments, the Escherichia bacteria is selected from the group including, but not limited to, E. albertii, E. fergusonii, E. hermannii, E. marmotae, and E. coli. In some embodiments, the Escherichia is E. coli. In some embodiments, the engineered bacteria or composition comprising the engineered bacteria may be administered to the subject by any effective route. In some embodiments, the route of administration is oral.

### Methods of Treating Small Intestine Bacterial Overgrowth or a Bacterial Infection

In some embodiments, a method of treating bacterial infection or bacterial overgrowth in a subject in need thereof is provided. In some embodiments the method comprising administering to the subject a therapeutically effective amount a therapeutic payload protein, wherein the therapeutic payload protein comprises lysozyme and is generated by an engineered bacterium genetically modified with a nucleic acid molecule encoding a recombinant fusion protein comprising a synthetic pre-protein signal peptide and the lysozyme, thereby treating the bacterial infection or bacterial overgrowth. In some embodiments, the synthetic signal peptide is a pre-protein signal peptide comprising an amino acid sequence represented by Formula I, SEQ ID NO: 1, 2, 3, or 4. In some embodiments, the synthetic pre-protein signal peptide comprises an amino acid sequence of represented by Formula I. In some embodiments, the synthetic pre-protein signal peptide comprises an amino acid sequence of SEQ ID NO: 1. In some embodiments, the synthetic pre-protein signal peptide comprises an amino acid sequence of SEQ ID NO: 2. In some embodiments, the synthetic pre-protein signal peptide comprises an amino acid sequence of SEQ ID NO: 3. In some embodiments, the synthetic pre-protein signal peptide comprises an amino acid sequence of SEQ ID NO: 4. In some embodiments, the engineered bacteria may be any Escherichia bacteria. In some embodiments, the Escherichia bacteria is a Escherichia bacteria as provided for herein. In some embodiments, the Escherichia bacteria is selected from the group including, but not limited to, E. albertii, E. fergusonii, E. hermannii, E. marmotae, and E. coli. In some embodiments, the Escherichia is E. coli. In some embodiments, the therapeutic payload protein or composition comprising the therapeutic payload protein may be administered to the subject by any effective route. In some embodiments, the route of administration is oral. The bacterial infection may be caused by be any gram-positive or gram-negative bacteria, such as, but not limited to, an infection of Escherichia coli (E. coli), Clostridioides difficile, P. aeruginosa, Shigella, Salmonella, Vibrio cholera, or cryptosporidium. In some embodiments, other antibacterial proteins may be produced by an engineered bacteria and therefore provide treatment for bacterial overgrowth or infection in a subject. In some embodiments, these other antibacterial proteins include, but are not limited to human beta defensins, peptide antimicrobials of animal origin (e.g., magainin, dermaseptin, cateslytin), and peptide antimicrobials of microbe origin (e.g., misin, sakacin). In any embodiment, a method of treating a bacterial infection with a therapeutic payload protein comprising lysozyme, generated by a bacteria as described herein, may comprise administering an antibacterial agent in combination with the lysozyme. For example, a bacterial infection may be treated by administering a therapeutically effective amount of lysozyme produced by the engineered bacteria as provided for herein and a therapeutically effective amount of an antibacterial agent, such as quinupristin, piperacillin, penicillin, clarithromycin, nitrofurantoin, ciprofloxacin, telithromycin, metronidazole, levofloxacin, erythromycin, theophylline, gemifloxacin, tetracycline, azithromycin, delafloxacin, eravacycline, moxifloxacin, dalbavancin, amoxicillin, fidaxomicin, tigecycline, ceftriaxone, minocycline, rifapentine, clindamycin, ceftazidime, oritayancin, norfloxacin, doxycycline, cefuroxime, tobramycin, ceftibuten, gentamicin, cefotaxime, vancomycin, telavancin, daptomycin, cephalexin, fofomycin, tedizolid, aztreonam, nafcillin, phenytoin, ertapenem, cefazolin, isoniazid, doripenem, rifabutin, meropenem, linezolid, oflaxacin, cefoxitin, oxacillin, warfarin, neomycin, rifampin, cefepime, and digoxin. The antibacterial agent can be administered by any route, such as oral, topical, intranasal, mucosal, otic, parenteral, or the like

In some embodiments, the method comprises administering to the subject a therapeutically effective amount of an engineered bacteria genetically modified to express a recombinant polypeptide comprising lysozyme and a synthetic pre-protein signal peptide, thereby treating the bacterial infection or overgrowth. In some embodiments, the pre-protein signal peptide comprises an amino acid sequence represented by Formula I, SEQ ID NO: 1, 2, 3, or 4. In some embodiments, the synthetic pre-protein signal peptide comprises an amino acid sequence of represented by Formula I. In some embodiments, the synthetic pre-protein signal peptide comprises an amino acid sequence of SEQ ID NO: 1. In some embodiments, the synthetic pre-protein signal peptide comprises an amino acid sequence of SEQ ID NO: 2. In some embodiments, the synthetic pre-protein signal peptide comprises an amino acid sequence of SEQ ID NO: 3. In some embodiments, the synthetic pre-protein signal peptide comprises an amino acid sequence of SEQ ID NO: 4. In some embodiments, the method comprises administering to the subject a composition comprising a therapeutically effective amount of an engineered bacteria genetically modified to express a recombinant fusion protein comprising lysozyme and a synthetic pre-protein signal peptide, thereby treating the bacterial infection or overgrowth. In some embodiments, the engineered bacteria may be any Escherichia bacteria. In some embodiments, the Escherichia bacteria is a Escherichia bacteria as provided for herein. In some embodiments, the Escherichia bacteria is selected from the group including, but not limited to, E. albertii, E. fergusonii, E. hermannii, E. marmotae, and E. coli. In some embodiments, the Escherichia is E. coli. The bacterial infection may be caused by be any gram-positive or gram-negative bacteria, such as, but not limited to, an infection of Escherichia Coli (E. coli), Clostridioides difficile, P. aeruginosa, Shigella, Salmonella, Vibrio cholera, or cryptosporidium. In some embodiments, other antibacterial proteins may be produced by an engineered bacteria and therefore provide treatment for bacterial overgrowth or infection in a subject. In some embodiments, these other antibacterial proteins include, but are not limited to human beta defensins, peptide antimicrobials of animal origin (e.g., magainin, dermaseptin, cateslytin), and peptide antimicrobials of microbe origin (e.g., misin, sakacin). In any embodiment, a method of treating a bacterial infection with engineered bacteria genetically modified to express lysozyme, as described herein, may comprise administering an antibacterial agent in combination with the engineered bacteria. For example, a bacterial infection may be treated by administering a therapeutically effective amount of engineered bacteria genetically modified to express a recombinant fusion protein comprising a synthetic signal peptide and lysozyme and a therapeutically effective amount of an antibacterial agent, such as quinupristin, piperacillin, penicillin, clarithromycin, nitrofurantoin, ciprofloxacin, telithromycin, metronidazole, levofloxacin, erythromycin, theophylline, gemifloxacin, tetracycline, azithromycin, delafloxacin, eravacycline, moxifloxacin, dalbavancin, amoxicillin, fidaxomicin, tigecycline, ceftriaxone, minocycline, rifapentine, clindamycin, ceftazidime, oritayancin, norfloxacin, doxycycline, cefuroxime, tobramycin, ceftibuten, gentamicin, cefotaxime, vancomycin, telavancin, daptomycin, cephalexin, fofomycin, tedizolid, aztreonam, nafcillin, phenytoin, ertapenem, cefazolin, isoniazid, doripenem, rifabutin, meropenem, linezolid, oflaxacin, cefoxitin, oxacillin, warfarin, neomycin, rifampin, cefepime, and digoxin. The antibacterial agent can be administered by any route, such as oral, topical, intranasal, mucosal, otic, parenteral, or the like.

### Methods of Treating Insulin Deficiency/Diabetes

An engineered bacteria may be used to treat an insulin deficiency or disorder, such as type 1 and type 2 diabetes mellitus. In some embodiments, the bacterium is used to produce a therapeutic payload protein useful for treating type 1 and type 2 diabetes mellitus. In some embodiments, the bacteria producing the therapeutic payload protein is useful for treating type 1 and type 2 diabetes mellitus. Therefore, in some embodiments, a method of treating type 1 or type 2 diabetes mellitus in a subject in need thereof is provided, the method comprising administering to the subject a therapeutically effective amount of an engineered bacteria genetically modified to express a recombinant polypeptide comprising insulin or an incretin (or a peptide analog or pro-drug thereof) and a synthetic pre-protein signal peptide, thereby treating the insulin deficiency or disorder. In some embodiments, a method of treating type 1 diabetes mellitus in a subject in need thereof is provided, the method comprising administering to the subject a therapeutically effective amount of an engineered bacteria genetically modified to express a recombinant polypeptide comprising insulin or an incretin (or a peptide analog or pro-drug thereof) and a synthetic pre-protein signal peptide, thereby treating type 1 diabetes mellitus. In some embodiments, a method of treating type 2 diabetes mellitus in a subject in need thereof is provided, the method comprising administering to the subject a therapeutically effective amount of an engineered bacteria genetically modified to express a recombinant polypeptide comprising insulin or an incretin (or a peptide analog or pro-drug thereof) and a synthetic pre-protein signal peptide, thereby treating type 2 diabetes mellitus. In some embodiments, the synthetic pre-protein signal peptide comprises an amino acid sequence represented by Formula I, SEQ ID NO: 1, 2, 3, or 4. In some embodiments, the synthetic pre-protein signal peptide comprises an amino acid sequence of represented by Formula I. In some embodiments, the synthetic pre-protein signal peptide comprises an amino acid sequence of SEQ ID NO: 1. In some embodiments, the synthetic pre-protein signal peptide comprises an amino acid sequence of SEQ ID NO: 2. In some embodiments, the synthetic pre-protein signal peptide comprises an amino acid sequence of SEQ ID NO: 3. In some embodiments, the synthetic pre-protein signal peptide comprises an amino acid sequence of SEQ ID NO: 4. Examples of suitable incretins, in any embodiment, include but are not limited to GLP-1, GLP-2, leptin, apelin, ghrelin, PYY, nesfatin, diaglutide, exenatide, liraglutide, semaglutide, sitagliptin, saxagliptin, alogliptin, linagliptin, and GIP. In some embodiments, the incretin is GLP-1. In some embodiments, the incretin is GLP-2. In some embodiments, the incretin is leptin. In some embodiments, the incretin is apelin. In some embodiments, the incretin is ghrelin. In some embodiments, the incretin is PYY. In some embodiments, the incretin is nesfatin. In some embodiments, the incretin is diaglutide. In some embodiments, the incretin is exenatide. In some embodiment, the incretin is liraglutide. In some embodiments, the incretin is semaglutide. In some embodiments, the incretin is sitagliptin. In some embodiments, the incretin is saxagliptin. In some embodiments, the incretin is alogliptin. In some embodiments, the incretin is linagliptin. In some embodiments, the incretin is GIP. In some embodiments, the engineered bacteria may be any Escherichia bacteria as provided for herein. In some embodiments, the Escherichia bacteria is selected from the group including, but not limited to, E. albertii, E. fergusonii, E. hermannii, E. marmotae, and E. coli. In some embodiments, the Escherichia is E. coli. In some embodiments, the engineered bacteria may be administered to the subject by any effective route. In some embodiments, the engineered bacterial is administered orally.

In some embodiments, the method of treating type 1 or type 2 diabetes mellitus in a subject in need thereof comprises administering to the subject a therapeutically effective amount a therapeutic payload protein, wherein the therapeutic payload protein is an insulin or an incretin (or a peptide analog or pro-drug thereof) and is generated by an engineered bacterium genetically modified with a nucleic acid molecule encoding a recombinant fusion protein comprising a synthetic pre-protein signal peptide and the protein, thereby treating the type 1 or type 2 diabetes mellitus. In some embodiments, the synthetic signal peptide is a pre-protein signal peptide comprising an amino acid sequence represented by Formula I, SEQ ID NO: 1, 2, 3, or 4. In some embodiments, the synthetic pre-protein signal peptide comprises an amino acid sequence of represented by Formula I. In some embodiments, the synthetic pre-protein signal peptide comprises an amino acid sequence of SEQ ID NO: 1. In some embodiments, the synthetic pre-protein signal peptide comprises an amino acid sequence of SEQ ID NO: 2. In some embodiments, the synthetic pre-protein signal peptide comprises an amino acid sequence of SEQ ID NO: 3. In some embodiments, the synthetic pre-protein signal peptide comprises an amino acid sequence of SEQ ID NO: 4. Examples of suitable incretins, in any embodiment, include but are not limited to GLP-1, GLP-2, leptin, apelin, ghrelin, PYY, nesfatin, diaglutide, exenatide, liraglutide, semaglutide, sitagliptin, saxagliptin, alogliptin, linagliptin, and GIP. In some embodiments, the incretin is GLP-1. In some embodiments, the incretin is GLP-2. In some embodiments, the incretin is leptin. In some embodiments, the incretin is apelin. In some embodiments, the incretin is ghrelin. In some embodiments, the incretin is PYY. In some embodiments, the incretin is nesfatin. In some embodiments, the incretin is diaglutide. In some embodiments, the incretin is exenatide. In some embodiment, the incretin is liraglutide. In some embodiments, the incretin is semaglutide. In some embodiments, the incretin is sitagliptin. In some embodiments, the incretin is saxagliptin. In some embodiments, the incretin is alogliptin. In some embodiments, the incretin is linagliptin. In some embodiments, the incretin is GIP. In some embodiments, the engineered bacteria may be any Escherichia bacteria. In some embodiments, the Escherichia bacteria is a Escherichia bacteria as provided for herein. In some embodiments, the Escherichia bacteria is selected from the group including, but not limited to, E. albertii, E. fergusonii, E. hermannii, E. marmotae, and E. coli. In some embodiments, the Escherichia is E. coli. In some embodiments, the therapeutic payload protein or composition comprising the therapeutic payload protein may be administered to the subject by any effective route. In some embodiments, the route of administration is oral.

### Methods of Repairing GI Epithelium

Engineered bacteria may be used to promote healing and repair of GI epithelium, for example, as caused by any disease or condition such as IBD or IBS, through the production of trefoil factors (e.g., TFF1/2/3) or IGF1. In some embodiments, the bacterium is used to produce a therapeutic payload protein useful for promoting healing and repair of GI epithelium. In some embodiments, the bacteria producing the therapeutic payload protein is useful for promoting healing and repair of GI epithelium. Therefore, in some embodiments, a method of promoting growth and repair in GI endothelium in a subject in need thereof is provided, the method comprising administering to the subject a therapeutically effective amount of engineered bacteria genetically modified to express a recombinant polypeptide comprising one or more of TFF1, TFF2, TFF3, or IGF1 and a synthetic pre-protein signal peptide, thereby promoting healing and repair of the GI epithelium. In some embodiments, the recombinant polypeptide comprises TFF1 and a synthetic pre-protein signal peptide. In some embodiments, the recombinant polypeptide comprises TFF2 and a synthetic pre-protein signal peptide. In some embodiments, the recombinant polypeptide comprises TFF3 and a synthetic pre-protein signal peptide. In some embodiments, the recombinant polypeptide comprises IGF1 and a synthetic pre-protein signal peptide. In some embodiments, the synthetic pre-protein signal peptide comprises an amino acid sequence represented by Formula I, SEQ ID NO: 1, 2, 3, or 4. In some embodiments, the synthetic pre-protein signal peptide comprises an amino acid sequence of represented by Formula I. In some embodiments, the synthetic pre-protein signal peptide comprises an amino acid sequence of SEQ ID NO: 1. In some embodiments, the synthetic pre-protein signal peptide comprises an amino acid sequence of SEQ ID NO: 2. In some embodiments, the synthetic pre-protein signal peptide comprises an amino acid sequence of SEQ ID NO: 3. In some embodiments, the synthetic pre-protein signal peptide comprises an amino acid sequence of SEQ ID NO: 4. In some embodiments, the engineered bacteria may be any Escherichia bacteria as provided for herein. In some embodiments, the Escherichia bacteria is selected from the group including, but not limited to, E. albertii, E. fergusonii, E. hermannii, E. marmotae, and E. coli. In some embodiments, the Escherichia is E. coli. In some embodiments, the engineered bacteria may be administered to the subject by any effective route. In some embodiments, the engineered bacteria is administered orally.

In some embodiments, the method of promoting growth and repair in GI endothelium in a subject in need thereof comprises administering to the subject a therapeutically effective amount a therapeutic payload protein, wherein the therapeutic payload protein comprises one or more of TFF1, TFF2, TFF3, or IGF1 and is generated by an engineered bacterium genetically modified with a nucleic acid molecule encoding a recombinant fusion protein comprising a synthetic pre-protein signal peptide and the protein, thereby promoting healing and repair of the GI epithelium. In some embodiments, the synthetic signal peptide is a pre-protein signal peptide comprising an amino acid sequence represented by Formula I, SEQ ID NO: 1, 2, 3, or 4. In some embodiments, the synthetic pre-protein signal peptide comprises an amino acid sequence of represented by Formula I. In some embodiments, the synthetic pre-protein signal peptide comprises an amino acid sequence of SEQ ID NO: 1. In some embodiments, the synthetic pre-protein signal peptide comprises an amino acid sequence of SEQ ID NO: 2. In some embodiments, the synthetic pre-protein signal peptide comprises an amino acid sequence of SEQ ID NO: 3. In some embodiments, the synthetic pre-protein signal peptide comprises an amino acid sequence of SEQ ID NO: 4. In some embodiments, the engineered bacteria may be any Escherichia bacteria. In some embodiments, the Escherichia bacteria is a Escherichia bacteria as provided for herein. In some embodiments, the Escherichia bacteria is selected from the group including, but not limited to, E. albertii, E. fergusonii, F. hermannii, F. marmotae, and E. coli. In some embodiments, the Escherichia is E. coli. In some embodiments, the therapeutic payload protein or composition comprising the therapeutic payload protein may be administered to the subject by any effective route. In some embodiments, the route of administration is oral.

### Methods of Treating Short Bowel Syndrome

Engineered bacteria may be used to treat short bowel syndrome. In some embodiments, the bacterium is used to produce a therapeutic payload protein useful for treating short bowel syndrome. In some embodiments, the bacteria producing the therapeutic payload protein is useful for treating short bowel syndrome. Therefore, in some embodiments, a method of treating short bowel syndrome in a subject in need thereof is provided, the method comprising administering to the subject a therapeutically effective amount of engineered bacteria genetically modified to express a recombinant polypeptide comprising IGF1, GLP-2 or any synthetic analog or prodrug thereof and a synthetic pre-protein signal peptide, thereby treating short bowel syndrome. In some embodiments, the recombinant polypeptide comprises IGF1 or a synthetic analog or prodrug thereof and a synthetic pre-protein signal peptide. In some embodiments, the recombinant polypeptide comprises GLP-2 or a synthetic analog or prodrug thereof and a synthetic pre-protein signal peptide. In some embodiments, the synthetic pre-protein signal peptide comprises an amino acid sequence represented by Formula I, SEQ ID NO: 1, 2, 3, or 4. In some embodiments, the synthetic pre-protein signal peptide comprises an amino acid sequence of represented by Formula I. In some embodiments, the synthetic pre-protein signal peptide comprises an amino acid sequence of SEQ ID NO: 1. In some embodiments, the synthetic pre-protein signal peptide comprises an amino acid sequence of SEQ ID NO: 2. In some embodiments, the synthetic pre-protein signal peptide comprises an amino acid sequence of SEQ ID NO: 3. In some embodiments, the synthetic pre-protein signal peptide comprises an amino acid sequence of SEQ ID NO: 4. In some embodiments, the engineered bacteria may be any Escherichia bacteria as provided for herein. In some embodiments, the Escherichia bacteria is selected from the group including, but not limited to, E. albertii, E. fergusonii, E. hermannii, E. marmotae, and E. coli. In some embodiments, the Escherichia is E. coli. In some embodiments, the engineered bacteria may be administered to the subject by any effective route. In some embodiments, the engineered bacteria is administered orally.

In some embodiments, the method of treating short bowel syndrome in a subject in need thereof comprises administering to the subject a therapeutically effective amount a therapeutic payload protein, wherein the therapeutic payload protein comprises IGF1, GLP-2 or any synthetic analog or prodrug thereof and is generated by an engineered bacterium genetically modified with a nucleic acid molecule encoding a recombinant fusion protein comprising a synthetic pre-protein signal peptide and the protein, thereby treating the short bowel syndrome. In some embodiments, the synthetic signal peptide is a pre-protein signal peptide comprising an amino acid sequence represented by Formula I, SEQ ID NO: 1, 2, 3, or 4. In some embodiments, the synthetic pre-protein signal peptide comprises an amino acid sequence of represented by Formula I. In some embodiments, the synthetic pre-protein signal peptide comprises an amino acid sequence of SEQ ID NO: 1. In some embodiments, the synthetic pre-protein signal peptide comprises an amino acid sequence of SEQ ID NO: 2. In some embodiments, the synthetic pre-protein signal peptide comprises an amino acid sequence of SEQ ID NO: 3. In some embodiments, the synthetic pre-protein signal peptide comprises an amino acid sequence of SEQ ID NO: 4. In some embodiments, the engineered bacteria may be any Escherichia bacteria. In some embodiments, the Escherichia bacteria is a Escherichia bacteria as provided for herein. In some embodiments, the Escherichia bacteria is selected from the group including, but not limited to, E. albertii, E. fergusonii, E. hermannii, E. marmotae, and E. coli. In some embodiments, the Escherichia is E. coli. In some embodiments, the therapeutic payload protein or composition comprising the therapeutic payload protein may be administered to the subject by any effective route. In some embodiments, the route of administration is oral.

### Method of Reducing Inflammation

Engineered bacteria may be used to produce pro-repair cytokines such as IL-10, IL-22, and/or TGFβ, which may be suitable for treating a variety of diseases and conditions. In some embodiments, the bacterium is used to produce a pro-repair cytokine, and the purified pro-repair cytokine is useful for reducing inflammation. In some embodiments, the bacteria producing the pro-repair cytokine is useful for reducing inflammation. Oral administration of IL-10, IL-22 and/or TGFβ may be beneficial for treating and repairing damage caused by inflammatory GI conditions, such as colitis, IBS. IBD, and the like. Therefore, in some embodiments, a method of repairing damage caused by inflammatory GI conditions in a subject in need thereof is provided, the method comprising administering to the subject a therapeutically effective amount of engineered bacteria genetically modified to express a recombinant polypeptide comprising one or more of IL-10, IL-22, and TGFβ or an analog or prodrug thereof and a synthetic pre-protein signal peptide. In some embodiments, the recombinant polypeptide comprises IL-10 or an analog or prodrug thereof and a synthetic pre-protein signal peptide. In some embodiments, the recombinant polypeptide comprises IL-22 or an analog or prodrug thereof and a synthetic pre-protein signal peptide. In some embodiments, the recombinant polypeptide comprises TGFβ or an analog or prodrug thereof and a synthetic pre-protein signal peptide. In some embodiments, the synthetic pre-protein signal peptide comprises an amino acid sequence represented by Formula I, SEQ ID NO: 1, 2, 3 or 4. In some embodiments, the synthetic pre-protein signal peptide comprises an amino acid sequence of represented by Formula I. In some embodiments, the synthetic pre-protein signal peptide comprises an amino acid sequence of SEQ ID NO: 1. In some embodiments, the synthetic pre-protein signal peptide comprises an amino acid sequence of SEQ ID NO: 2. In some embodiments, the synthetic pre-protein signal peptide comprises an amino acid sequence of SEQ ID NO: 3. In some embodiments, the synthetic pre-protein signal peptide comprises an amino acid sequence of SEQ ID NO: 4. In some embodiments, the engineered bacteria may be any Escherichia bacteria as provided for herein. In some embodiments, the Escherichia bacteria is selected from the group including, but not limited to, E. albertii, E. fergusonii, E. hermannii, E. marmotae, and E. coli. In some embodiments, the Escherichia is E. coli. In some embodiments, the engineered bacteria may be administered to the subject by any effective route. In some embodiments, the engineered bacteria is administered orally.

In some embodiments, the method of repairing damage caused by inflammatory GI conditions in a subject in need thereof comprises administering to the subject a therapeutically effective amount a therapeutic payload protein, wherein the therapeutic payload protein comprises one or more of IL-10, IL-22, and TGFβ or an analog or prodrug thereof and is generated by an engineered bacterium genetically modified with a nucleic acid molecule encoding a recombinant fusion protein comprising a synthetic pre-protein signal peptide and the protein, thereby repairing damage caused by inflammatory GI conditions. In some embodiments, the synthetic signal peptide is a pre-protein signal peptide comprising an amino acid sequence represented by Formula I, SEQ ID NO: 1, 2, 3, or 4. In some embodiments, the synthetic pre-protein signal peptide comprises an amino acid sequence of represented by Formula I. In some embodiments, the synthetic pre-protein signal peptide comprises an amino acid sequence of SEQ ID NO: 1. In some embodiments, the synthetic pre-protein signal peptide comprises an amino acid sequence of SEQ ID NO: 2. In some embodiments, the synthetic pre-protein signal peptide comprises an amino acid sequence of SEQ ID NO: 3. In some embodiments, the synthetic pre-protein signal peptide comprises an amino acid sequence of SEQ ID NO: 4. In some embodiments, the engineered bacteria may be any Escherichia bacteria. In some embodiments, the Escherichia bacteria is a Escherichia bacteria as provided for herein. In some embodiments, the Escherichia bacteria is selected from the group including, but not limited to, E. albertii, F. fergusonii, F. hermannii, F. marmotae, and E. coli. In some embodiments, the Escherichia is E. coli. In some embodiments, the therapeutic payload protein or composition comprising the therapeutic payload protein may be administered to the subject by any effective route. In some embodiments, the route of administration is oral.

Agricultural Compositions and Methods of their Use

An engineered bacteria may be used to produce agricultural payload proteins such as, but not limited to, decomposition enzymes (e.g., cellulose), soil and other agricultural enzymes (e.g., lipases, proteases, polymerases, amylases, peroxidases, catalases, beta glucosidase. FDA hydrolysis, amidase, urease, phosphatase, sulfatase), fungicides (e.g., chitinase, chitin-binding proteins, cyclophilin-like proteins, defensins, lipid transfer proteins, miraculin-like proteins, nucleases, thaumatin-like proteins, and the like), insecticides (e.g., Vip1, Vip2, Vip3, Cry proteins, and the like), plant activators (e.g., branched-β-glucans, chitin oligomers, pectolytic enzymes, elicitor activity independent from enzyme activity (e.g. endoxylanase, elicitins, PaNie), avr gene products (e.g., AVR4, AVR9), viral proteins (e.g., vial coat protein, Harpins), flagellin, protein or peptide toxin (e.g., victorin), glycoproteins, glycopeptide fragments of invertase, syringolids, Nod factors (lipochitoolingo-saccharides), FACs (fatty acid amino acid conjugates), ergosterol, bacterial toxins (e.g., coronatine), and sphinganine analogue mycotoxins (e.g., fumonisin B1), which may be suitable for treating a variety of diseases and conditions. Application of one or more of the above described agricultural payload proteins to an agricultural environment, such as a crop, garden, or the like, may be beneficial for promoting soil and plant health. Therefore, in some embodiments, a method of promoting soil and/or plant health is provided, the method comprising applying to the soil or plant an agriculturally effective amount of an engineered bacteria genetically modified to express a recombinant polypeptide comprising one or more of an agricultural payload protein and synthetic signal peptide, thereby promoting soil and/or plant health. In some embodiments, the synthetic signal peptide comprises a pre-protein amino acid sequence represented by Formula I, SEQ ID NO: 1, 2, 3, or 4. In some embodiments, the synthetic pre-protein signal peptide comprises an amino acid sequence of represented by Formula I. In some embodiments, the synthetic pre-protein signal peptide comprises an amino acid sequence of SEQ ID NO: 1. In some embodiments, the synthetic pre-protein signal peptide comprises an amino acid sequence of SEQ ID NO: 2. In some embodiments, the synthetic pre-protein signal peptide comprises an amino acid sequence of SEQ ID NO: 3. In some embodiments, the synthetic pre-protein signal peptide comprises an amino acid sequence of SEQ ID NO: 4. In some embodiments, the engineered bacteria may be any Escherichia bacteria as provided for herein. In some embodiments, the Escherichia bacteria is selected from the group including, but not limited to, E. albertii, E. fergusonii, E. hermannii, E. marmotae, and E. coli. In some embodiments, the Escherichia is E. coli. In some embodiments, the engineered bacteria may be applied to soil or plants via any known method. In some embodiments, the engineered bacteria are applied to the soil or plants via a method as provided for below.

The engineered bacteria, as described herein may be incorporated into a composition comprising a formulation inert or other formulation ingredient, such as polysaccharides (starches, maltodextrins, methylcelluloses, proteins, such as whey protein, peptides, gums), sugars (lactose, trehalose, sucrose), lipids (lecithin, vegetable oils, mineral oils), salts (sodium chloride, calcium carbonate, sodium citrate), and silicates (clays, amorphous silica, fumed/precipitated silicas, silicate salts). In some embodiments, such as those in which the compositions are applied to soil, a composition may comprise a carrier, such as water or a mineral or organic material such as peat that facilitates incorporation of the compositions into the soil. In some embodiments, such as those in which the composition is used for seed treatment or as a root dip, the carrier is a binder or sticker that facilitates adherence of the composition to the seed or root. In another embodiment in which the compositions are used as a seed treatment the formulation ingredient is a colorant. In other compositions, the formulation ingredient is a preservative. Suitable composition may comprise about 1×10² to about 1×10¹⁰ cfu/g of the engineered bacteria, such as at least 1×10⁶ cfu/g, at least 1×10⁷ cfu/g, at least 1×10⁸ cfu/g, or at least 1×10⁹ cfu/g.

The engineered bacteria and compositions thereof disclosed herein may be used to treat a wide variety of agricultural and/or horticultural crops, including those grown for seed, produce, landscaping and those grown for seed production. Representative plants that can be treated using the compositions disclosed herein include but are not limited to the following: brassica, bulb vegetables, cereal grains, citrus, cotton, cucurbits, fruiting vegetables, leafy vegetables, legumes, oil seed crops, peanut, pome fruit, root vegetables, tuber vegetables, corn vegetables, stone fruit, tobacco, strawberry and other berries, and various ornamentals. Representative plants include but are not limited to the following monocots and dicots; bulb vegetables; cereal grains (such as wheat, barley, rice); corn (maize), citrus fruits (such as grapefruit, lemon, and orange); cotton and other fiber crops, cucurbits; fruiting vegetables; leafy vegetables (such as celery, head and leaf lettuce, and spinach); legumes (such as soy beans, green beans, chick peas, lentils); oil seed crops; peanut; pome frit (such as apple and pear); stone fruits (such as almond, pecan, and walnut); root vegetables; tuber vegetables; corn vegetables; tobacco, strawberry and other berries; cole crops (such as broccoli, cabbage); grape; plants used for biomass production (such as miscanthus bamboo), pineapple; and flowering plants, bedding plants, and ornamentals (such as fern and hosta). Engineered bacteria and compositions thereof as disclosed herein may also be used to treat perennial plants, including plantation crops such as banana and coffee and those present in forests parks or landscaping.

Engineered bacteria and compositions thereof disclosed herein may be used to control plant parasitic nematodes, such as, but not limited to, root-knot, cyst, lesion and ring nematodes, including Meloidogyne spp., Heterodera spp., Globodera spp., Pratylenchus spp. and Criconemella sp. In some embodiments, the targets are root knot nematodes, such as M. incognita (cotton root knot nematode), M. javanica (Javanese root knot nematode), M. hapla (Northern root knot nematode), and M. arenaria (peanut root knot nematode). Accordingly, in some embodiments, a method of controlling, preventing or reducing a nematode infestation in an agricultural setting is provided. In some embodiments, the method comprises administering to the agricultural setting an effective amount of an engineered bacteria genetically modified to express a recombinant polypeptide comprising one or more of an agricultural payload protein and synthetic signal peptide, thereby preventing or reducing the nematode infestation. In some embodiments, the agricultural payload protein is a nematicide. In some embodiments, the synthetic signal peptide comprises a pre-protein amino acid sequence represented by Formula I, SEQ ID NO: 1, 2, 3, or 4. In some embodiments, the synthetic pre-protein signal peptide comprises an amino acid sequence of represented by Formula I. In some embodiments, the synthetic pre-protein signal peptide comprises an amino acid sequence of SEQ ID NO: 1. In some embodiments, the synthetic pre-protein signal peptide comprises an amino acid sequence of SEQ ID NO: 2. In some embodiments, the synthetic pre-protein signal peptide comprises an amino acid sequence of SEQ ID NO: 3. In some embodiments, the synthetic pre-protein signal peptide comprises an amino acid sequence of SEQ ID NO: 4. In some embodiments, the engineered bacteria may be any Escherichia bacteria as provided for herein. In some embodiments, the Escherichia bacteria is selected from the group including, but not limited to, E. albertii, E. fergusonii, E. hermannii, E. marmotae, and E. coli. In some embodiments, the Escherichia is E. coli. In some embodiments, the engineered bacteria may be applied to soil or plants via any known method. In some embodiments, the engineered bacteria are applied to the soil or plants via a method as provided for herein.

In some embodiments, engineered bacteria and compositions thereof may be used to control fungal infections in an agricultural environment. Accordingly, in some embodiments, a method of controlling, preventing or reducing a fungal infestation in an agricultural setting is provided. In some embodiments, the method comprises administering to the agricultural setting an effective amount of an engineered bacteria genetically modified to express a recombinant polypeptide comprising one or more of an agricultural payload protein and synthetic signal peptide, thereby controlling, preventing or reducing the fungal infestation. In some embodiments, the agricultural payload protein is a fungicide. In some embodiments, the fungicide is selected from the group including, but not limited to chitinase, chitin-binding proteins, cyclophilin-like proteins, defensins, lipid transfer proteins, miraculin-like proteins, nucleases, thaumatin-like proteins, and the like. In some embodiments, the fungicide is any appropriate fungicide. In some embodiments, the synthetic signal peptide comprises a pre-protein amino acid sequence represented by Formula I, SEQ ID NO: 1, 2, 3, or 4. In some embodiments, the synthetic pre-protein signal peptide comprises an amino acid sequence of represented by Formula I. In some embodiments, the synthetic pre-protein signal peptide comprises an amino acid sequence of SEQ ID NO: 1. In some embodiments, the synthetic pre-protein signal peptide comprises an amino acid sequence of SEQ ID NO: 2. In some embodiments, the synthetic pre-protein signal peptide comprises an amino acid sequence of SEQ ID NO: 3. In some embodiments, the synthetic pre-protein signal peptide comprises an amino acid sequence of SEQ ID NO: 4. In some embodiments, the engineered bacteria may be any Escherichia bacteria as provided for herein. In some embodiments, the Escherichia bacteria is selected from the group including, but not limited to, E. albertii, E. fergusonii, E. hermannii, E. marmotae, and E. coli. In some embodiments, the Escherichia is E. coli. In some embodiments, the engineered bacteria may be applied to soil or plants via any known method. In some embodiments, the engineered bacteria are applied to the soil or plants via a method as provided for herein.

In some embodiments, engineered bacteria and compositions thereof may be used to control, prevent, or reduce an insect or pest infestation in an agricultural environment. Accordingly, in some embodiments, a method of controlling, preventing or reducing an insect or pest infestation in an agricultural setting is provided. In some embodiments, the method comprises administering to the agricultural setting an effective amount of an engineered bacteria genetically modified to express a recombinant polypeptide comprising one or more of an agricultural payload protein and synthetic signal peptide, thereby preventing or reducing the insect or pest infestation. In some embodiments, the agricultural payload protein is a pesticide or an insecticide. In some embodiments, the insecticide is selected from the group including, but not limited to, Vip1, Vip2, Vip3, Cry proteins, and the like. In some embodiments, the insecticide is any appropriate insecticide. In some embodiments, the pesticide is any appropriate pesticide. In some embodiments, the synthetic signal peptide comprises a pre-protein amino acid sequence represented by Formula I, SEQ ID NO: 1, 2, 3, or 4. In some embodiments, the synthetic pre-protein signal peptide comprises an amino acid sequence of represented by Formula I. In some embodiments, the synthetic pre-protein signal peptide comprises an amino acid sequence of SEQ ID NO: 1. In some embodiments, the synthetic pre-protein signal peptide comprises an amino acid sequence of SEQ ID NO: 2. In some embodiments, the synthetic pre-protein signal peptide comprises an amino acid sequence of SEQ ID NO: 3. In some embodiments, the synthetic pre-protein signal peptide comprises an amino acid sequence of SEQ ID NO: 4. In some embodiments, the engineered bacteria may be any Escherichia bacteria as provided for herein. In some embodiments, the Escherichia bacteria is selected from the group including, but not limited to, E. albertii, E. fergusonii, E. hermannii, E. marmotae, and E. coli. In some embodiments, the Escherichia is E. coli. In some embodiments, the engineered bacteria may be applied to soil or plants via any known method. In some embodiments, the engineered bacteria are applied to the soil or plants via a method as provided for herein.

Engineered bacteria and compositions thereof disclosed herein may be used to enhance plant health (such as by promoting plant health, enhancing resistance to abiotic stress, or improving plant vigor) and/or control a plant disease and/or control a plant pest. In some embodiments, the method of promoting plant health comprises applying one or more of the engineered bacteria or compositions thereof to the plant, to a part of the plant and/or to the locus surrounding the plant, such as to a plant's growth media. Thus, in some embodiments, the method of promoting plant health comprises applying the engineered bacteria or a composition thereof to the soil. For example, the composition can be applied before, during or after the plant or plant part comes into contact with the soil. As further examples, the methods include but are not limited to applying the composition using an application method such as soil surface drench, shanking in, injection, chemigation, or application in-furrow.

When used as a soil treatment, the engineered bacteria and compositions thereof, as disclosed herein, may be applied as a soil surface drench, shanked-in, injected and/or applied in-furrow or by mixture with irrigation water. The rate of application for drench soil treatments, which may be applied at planting, during or after seeding, or after transplanting and at any stage of plant growth, may be about 4×10¹¹ to about 8×10¹² cfu per acre, such as about 1×10¹² to about 6×10¹² cfu per acre. The rate of application for in-furrow treatments, applied at planting, is about 2.5×10¹⁰ to about 5×10¹¹ cfu per 1000 row feet, such about 6×10¹⁰ to about 4×10¹¹ cfu per 1000 row feet. Those of skill in the art will understand how to adjust rates for broadcast treatments (where applications are at a lower rate but made more often) and other less common soil treatments. Such adjustments are within the scope of the present application.

In any embodiment disclosed herein, the engineered bacteria and compositions thereof, as described herein, may be mixed with other chemical and non-chemical additives, adjuvants and/or treatments, wherein such treatments include but are not limited to chemical and non-chemical fungicides, insecticides, miticides, nematicides, fertilizers, nutrients, minerals, auxins, growth stimulants, and the like.

### ENUMERATED EMBODIMENTS

In some embodiments, the following embodiments are provided:
1. A pre-protein signal peptide comprising an amino acid sequence of Formula I,
wherein Formula I is represented as:

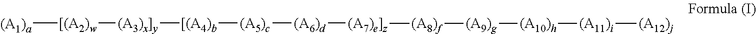

wherein:
   each w is, independently, 1 or 2;
   each x is, independently, 0 or 1;
   y is 1, 2, or 3;
   z is 2, 3, 4, 5, 6, 7, 8, 9, 10, 11, or 12;
   each b, c, d, and e are each, independently, 0 or 1; and
   a, f, g, h, i, and j are each, independently, 0 or 1,
wherein:
   A₁ is methionine;
   each A₂ is, independently, an amino acid selected from the group consisting of K, R, N, A, P, S, T, I, and F;
   each A₃ is, independently, an amino acid selected from the group consisting of I, L, F, V, M, Y, and H;
   each A₄ is, independently, an amino acid selected from the group consisting of L, V, C, A, F, I, T, M, P, S, G, W, Y, Q, N, R, and H;
   each A₅ is, independently, an amino acid selected from the group consisting of A, G, S, T, P, M, C, V, W, I, L, F, Y, Q, N, R, E, K, D, and H;
   each A₆ is, independently, an amino acid selected from the group consisting of G, S, N, and Q;
   each A₇ is, independently, an amino acid selected from the group consisting of S, N, T, G, V, I, Q, A, C, P, Y, M, F, and L;
   A₈ is an amino acid selected from the group consisting of A, T, G, S, P, V, I, L, Q, R, M, W, F, C, N, K, E, D, and H;
   A₉ is an amino acid selected from the group consisting of Q, F, N, S, E, T, D, R, H, K, G, A, P, Y, M, V, W, I, and L;
   A₁₀ is an amino acid selected from the group consisting of A, T, G, S, P, V, I, L, Q, R, M, W, F, C, N, K, E, D, and H;
   A₁₁ is an amino acid selected from the group consisting of A, T, G, S, P, V, I, L, Q, R, M, W, F, C, N, K, E, D, and H; and
   A₁₂ is an amino acid selected from the group consisting of D, E, Q, N, S, H, T, R, K, G, A, C, Y, P, M, V, W, I, and L.
2 The pre-protein signal peptide of embodiment 1, wherein
   A₁ is methionine;
   each A₂ is, independently, an amino acid selected from the group consisting of K, R, and N;
   each A₃ is, independently, isoleucine (I);
   each A₄ is, independently, an amino acid selected from the group consisting of L, V, C, and A;
   each A₅ is, independently, an amino acid selected from the group consisting of A, G, and S;
   each A₆ is, independently, an amino acid selected from the group consisting of G, S, N, and Q;
   each A₇ is, independently, an amino acid selected from the group consisting of S, N, T, G, V, and I;
   A₈ is an amino acid selected from the group consisting of A, T, G, and S;
   A₉ is an amino acid selected from the group consisting of Q, and F;
   A₁₀ is an amino acid selected from the group consisting of A, T, G, and S;
   A₁₁ is an amino acid selected from the group consisting of A, T, G, and S; and/or
   A₁₂ is an amino acid selected from the group consisting of D, E, Q, N, S, H, T, R, K, G, A, C, Y, P, M, V, W, I, and L.
3. The pre-protein signal peptide of embodiment 1 or embodiment 2, wherein the pre-protein signal peptide comprises a minimum number of amino acids, a maximum number of amino acids, or both a minimum and maximum number of amino acids.
4. The pre-protein signal peptide of any one of embodiments 1-3, wherein the pre-protein signal peptide comprises a minimum of 15 amino acids.
5 The pre-protein signal peptide of any one of embodiments 1-3, wherein the pre-protein signal peptide comprises a maximum of 45 amino acids.
6. The pre-protein signal peptide of any one of embodiments 1-3, wherein the pre-protein signal peptide comprises a minimum of 15 amino acids and a maximum of 45 amino acids.
7. The pre-protein signal peptide of embodiment 1 or embodiment 2, wherein the signal peptide comprises an amino acid sequence having at least 90%, 91%, 92%, 93%, 94%, 95%, 96%, 97%, 98%, or 99% identity to an amino acid sequence of SEQ ID NO: 1, 2, 3, or 4.
8. The pre-protein signal peptide of embodiment 1 or embodiment 2, wherein the signal peptide comprises an amino acid sequence having at least 95% identity to an amino acid sequence of SEQ ID NO: 1.
9. The pre-protein signal peptide of embodiment 8, wherein the signal peptide comprising an amino acid sequence having at least 95% identity to an amino acid sequence of SEQ ID NO: 1 comprises an amino acid sequence of any one of SEQ ID NOs: 73-NO: 84.
10. The pre-protein signal peptide of embodiment 1 or embodiment 2, wherein the signal peptide comprises an amino acid sequence having at least 95% identity to an amino acid sequence of SEQ ID NO: 2.
11. The pre-protein signal peptide of embodiment 10, wherein the signal peptide comprising an amino acid sequence having at least 95% identity to an amino acid sequence of SEQ ID NO: 2 comprises an amino acid sequence of SEQ ID NO: 85 or SEQ ID NO: 86.
12. The pre-protein signal peptide of embodiment 1 or embodiment 2, wherein the signal peptide comprises an amino acid sequence having at least 95% identity to an amino acid sequence of SEQ ID NO: 3.
13. The pre-protein signal peptide of embodiment 12, wherein the signal peptide comprising an amino acid sequence having at least 95% identity to an amino acid sequence of SEQ ID NO: 3 comprises an amino acid sequence of any one of SEQ ID NOs: 87-138.
14. The pre-protein signal peptide of embodiment 1 or embodiment 2, wherein the signal peptide comprises an amino acid sequence having at least 95% identity to an amino acid sequence of SEQ ID NO: 4.
15. The pre-protein signal peptide of embodiment 14, wherein the signal peptide comprising an amino acid sequence having at least 95% identity to an amino acid sequence of SEQ ID NO: 4 comprises an amino acid sequence of any one of SEQ ID NOs: 139-217.
16. The pre-protein signal peptide of embodiment 1 or embodiment 2, wherein the signal peptide comprises an amino acid sequence of SEQ ID NO: 1, 2, 3, or 4.
17. The pre-protein signal peptide of any one of embodiments 1-16, wherein the pre-protein signal peptide increases the secretion of a payload protein as compared to native signal peptides.
18. A recombinant polypeptide comprising a formula of X₁—Z₁ wherein:
   X₁ is a pre-protein signal peptide, and
   Z₁ is a payload protein.
19. The recombinant polypeptide of embodiment 18, wherein X₁ comprises an amino acid sequence of Formula I, wherein Formula I is represented as:

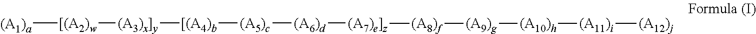

wherein:
   each w is, independently, 1 or 2;
   each x is, independently, 0 or 1;
   y is 1, 2, or 3;
   z is 2, 3, 4, 5, 6, 7, 8, 9, 10, 11, or 12;
   each b, c, d, and e are each, independently, 0 or 1; and
   a, f, g, h, i, and j are each, independently, 0 or 1,
wherein:
   A₁ is methionine
   each A₂ is, independently, an amino acid selected from the group consisting of K, R, N, A, P, S, T, I, and F;
   each A₃ is, independently, an amino acid selected from the group consisting of I, L, F, V, M, Y, and H;
   each A₄ is, independently, an amino acid selected from the group consisting of L, V, C, A, F, I, T, M, P, S, G, W, Y, Q, N, R, and H;
   each A₅ is, independently, an amino acid selected from the group consisting of A, G, S, T, P, M, C, V, W, I, L, F, Y, Q, N, R, E, K, D, and H;
   each A₆ is, independently, an amino acid selected from the group consisting of G, S, N, and Q;
   each A₇ is, independently, an amino acid selected from the group consisting of S, N, T, G, V, I, Q, A, C, P, Y, M, F, and L;
   A₈ is an amino acid selected from the group consisting of A, T, G, S, P, V, I, L, Q, R, M, W, F, C, N, K, E, D, and H;
   A₉ is an amino acid selected from the group consisting of Q, F, N, S, E, T, D, R, H, K, G, A, P, Y, M, V, W, I, and L;
   A₁₀ is an amino acid selected from the group consisting of A, T, G, S, P, V, I, L, Q, R, M, W, F, C, N, K, E, D, and H;
   A₁₁ is an amino acid selected from the group consisting of A, T, G, S, P, V, I, L, Q, R, M, W, F, C, N, K, E, D, and H; and
   A₁₂ is an amino acid selected from the group consisting of D, E, Q, N, S, H, T, R, K, G, A, C, Y, P, M, V, W, I, and L,
20. The recombinant polypeptide of claim 19, wherein
   A₁ is methionine;
   each A₂ is, independently, an amino acid selected from the group consisting of K, R, and N;
   each A₃ is, independently, isoleucine (I);
   each A₄ is, independently, an amino acid selected from the group consisting of L, V, C, and A;
   each A₅ is, independently, an amino acid selected from the group consisting of A, G, and S;
   each A₆ is, independently, an amino acid selected from the group consisting of G, S, N, and Q;
   each A₇ is, independently, an amino acid selected from the group consisting of S, N, T, G, V, and I;
   A₈ is an amino acid selected from the group consisting of A, T, G, and S;
   A₉ is an amino acid selected from the group consisting of Q, and F;
   A₁₀ is an amino acid selected from the group consisting of A, T, G, and S;
   A₁₁ is an amino acid selected from the group consisting of A, T, G, and S; and/or
   A₁₂ is an amino acid selected from the group consisting of D, E, Q, N, S, H, T, R, K, G, A, C, Y, P, M, V, W, I, and L.
21. The recombinant polypeptide of embodiment 19 or embodiment 20, wherein the pre-protein signal peptide X₁ given by Formula I comprises a minimum number of amino acids, a maximum number of amino acids, or both a minimum and maximum number of amino acids.
22. The recombinant polypeptide of any one of embodiments 19-21, wherein the pre-protein signal peptide X₁ given by Formula I comprises a minimum of 15 amino acids.
23. The recombinant polypeptide of any one of embodiments 19-21, wherein the pre-protein signal peptide X₁ given by Formula I comprises a maximum of 45 amino acids.
24. The recombinant polypeptide of any one of embodiments 19-21, wherein the pre-protein signal peptide X₁ given by Formula I comprises a minimum of 15 amino acids and a maximum of 45 amino acids.
25. The recombinant polypeptide of any one of embodiments 18-24, wherein X₁ comprises an amino acid sequence having at least 90%, 91%, 92%, 93%, 94%, 95%, 96%, 97%, 98%, 99%, or 100% identity to an amino acid sequence of SEQ ID NO: 1, 2, 3, or 4.
26. The recombinant polypeptide of any one of embodiments 18-25, wherein Z₁ is selected from the group consisting of an antiviral, insulin, an incretin, an enzyme, an enzyme inhibitor, a hormone, a cytokine, an antibody, a single domain antibody fragment, a single chain variable antibody fragment, an antimicrobial peptide, a mucosal protein, pesticide, bactericide herbicide, fungicide, nematicide, miticide, plant growth regulator, plant growth stimulator or fertilizer, a vaccine, a diagnostic protein, a feed conversion enzyme, a flavoring, a nutritional protein, venom peptides, endoglucanase, restriction enzymes, human growth hormone, Beta-lactamase, luciferase (such as, but not limited to, Renilla luciferase or NanoLuc luciferase), phytase, cellulase, chitinase, phosphatase, catalase, urokinase, tissue plasminogen activator, apolipoprotein, beta-glucosidases, hemicellulases, lignocellulose oxireductases, DNAses, NADPH dehydrogenase, alcohol oxidase, pyruvate decarboxylase, formolase, alpha-ketoglutarate dehydrogenase, branched chain alpha-ketoacid decarboxylase, copper radical oxidase, galactose oxidase, glycerol oxidase, amine oxidase, glyoxalase, amino monoaxidase, ethylene glycol oxidase, alditol oxidase, or 2-oxoglutarate dehydrogenase.
27. The recombinant polypeptide of any one of embodiments 18-25, wherein Z₁ is selected from the group consisting of an antiviral, insulin, an incretin, an enzyme, an enzyme inhibitor, a hormone, a cytokine, an antibody, a single domain antibody fragment, a single chain variable antibody fragment, an antimicrobial peptide, a mucosal protein, pesticide, bactericide herbicide, fungicide, nematicide, miticide, plant growth regulator, plant growth stimulator or fertilizer, a vaccine, a diagnostic protein, a feed conversion enzyme, a flavoring, or a nutritional protein.
28. The recombinant polypeptide of any one of embodiments 18-25, wherein Z₁ is selected from the group consisting of venom peptides, endoglucanase, restriction enzymes, human growth hormone, Beta-lactamase, luciferase (such as, but not limited to, Renilla luciferase or NanoLuc luciferase), phytase, cellulase, chitinase, phosphatase, catalase, urokinase, tissue plasminogen activator, apolipoprotein, beta-glucosidases, hemicellulases, lignocellulose oxireductases, DNAses, NADPH dehydrogenase, alcohol oxidase, pyruvate decarboxylase, formolase, alpha-ketoglutarate dehydrogenase, branched chain alpha-ketoacid decarboxylase, copper radical oxidase, galactose oxidase, glycerol oxidase, amine oxidase, glyoxalase, amino monoaxidase, ethylene glycol oxidase, alditol oxidase, or 2-oxoglutarate dehydrogenase.
29. The recombinant polypeptide of any one of embodiments 18-25, wherein Z₁ comprises an amino acid sequence having at least 90%, 91%, 92%, 93%, 94%, 95%, 96%, 97%, 98%, 99%, or 100% identity to an amino acid sequence of SEQ ID NO: 28, 29, 30, 31, 32, 33, 34, 35, 36, 37, 38, 39, 40, 41, 42, 43, 44, or 72.
30. The recombinant polypeptide of any one of embodiments 19-29, wherein the pre-protein signal peptide X₁ increases the secretion of the payload protein Z₁ as compared to native
31. An engineered bacterium comprising a heterologous nucleic acid molecule encoding a polypeptide having a formula of X₁—Z₁, wherein:
   X₁ is a pre-protein signal peptide of any one of embodiments 1-17, and
   Z₁ is a payload protein.
32. The engineered bacterium of embodiment 31, wherein the bacteria are Escherichia bacteria.
33. The engineered bacterium of embodiment 31 or 32, wherein the bacteria is selected from the group consisting of E. alberii, E. fergusonii, E. hermannii, E. marmotae, and E. coli.
34. The engineered bacterium of embodiment 31 or 32, wherein the bacteria is E. coli.
35. The engineered bacterium of any one of embodiments 31-34, wherein Z₁ is selected from the group consisting of an antiviral, insulin, an incretin, an enzyme, an enzyme inhibitor, a hormone, a cytokine, an antibody, a single domain antibody fragment, a single chain variable antibody fragment, an antimicrobial peptide, a mucosal protein, pesticide, bactericide herbicide, fungicide, nematicide, miticide, plant growth regulator, plant growth stimulator or fertilizer, a vaccine, a diagnostic protein, a feed conversion enzyme, a flavoring, a nutritional protein, venom peptides, endoglucanase, restriction enzymes, human growth hormone, Beta-lactamase, luciferase (such as, but not limited to, Renilla luciferase or NanoLuc luciferase), phytase, cellulase, chitinase, phosphatase, catalase, urokinase, tissue plasminogen activator, apolipoprotein, beta-glucosidases, hemicellulases, lignocellulose oxireductases, DNAses, NADPH dehydrogenase, alcohol oxidase, pyruvate decarboxylase, formolase, alpha-ketoglutarate dehydrogenase, branched chain alpha-ketoacid decarboxylase, copper radical oxidase, galactose oxidase, glycerol oxidase, amine oxidase, glyoxalase, amino monoaxidase, ethylene glycol oxidase, alditol oxidase, or 2-oxoglutarate dehydrogenase.
36. The engineered bacterium of any one of embodiments 31-34, wherein Z₁ is selected from the group consisting of an antiviral, insulin, an incretin, an enzyme, an enzyme inhibitor, a hormone, a cytokine, an antibody, a single domain antibody fragment, a single chain variable antibody fragment, an antimicrobial peptide, a mucosal protein, pesticide, bactericide herbicide, fungicide, nematicide, miticide, plant growth regulator, plant growth stimulator or fertilizer, a vaccine, a diagnostic protein, a feed conversion enzyme, a flavoring, or a nutritional protein
37. The engineered bacterium of any one of embodiments 31-34, wherein Z₁ is selected from the group consisting of venom peptides, endoglucanase, restriction enzymes, human growth hormone. Beta-lactamase, luciferase (such as, but not limited to, Renilla luciferase or NanoLuc luciferase), phytase, cellulase, chitinase, phosphatase, catalase, urokinase, tissue plasminogen activator, apolipoprotein, beta-glucosidases, hemicellulases, lignocellulose oxireductases, DNAses, NADPH dehydrogenase, alcohol oxidase, pyruvate decarboxylase, formolase, alpha-ketoglutarate dehydrogenase, branched chain alpha-ketoacid decarboxylase, copper radical oxidase, galactose oxidase, glycerol oxidase, amine oxidase, glyoxalase, amino monoaxidase, ethylene glycol oxidase, alditol oxidase, or 2-oxoglutarate dehydrogenase.
38. The engineered bacterium of any one of embodiments 31-34, wherein Z₁ comprises an amino acid sequence having at least 90%, 91%, 92%, 93%, 94%, 95%, 96%, 97%, 98%, 99%, or 100% identity to an amino acid sequence of SEQ ID NO: 28, 29, 30, 31, 32, 33, 34, 35, 36, 37, 38, 39, 40, 41, 42, 43, 44, or 72.
39. The engineered bacterium of any one of embodiments 31-38, wherein the pre-protein signal peptide X₁ increases the expression of the payload protein Z₁ as compared to native signal peptides.
40. A method for producing a payload protein, comprising:
   i) transfecting a bacterium with a nucleic acid molecule encoding for the recombinant polypeptide of any one of embodiments 18-30 to produce an engineered bacterium comprising the nucleic acid molecule;
   ii) culturing the engineered bacteria comprising the nucleic acid molecule under conditions sufficient to grow the bacteria, and
   iii) inducing secretion of the payload protein by the engineered bacteria.
41. The method of embodiment 40, wherein inducing secretion of the payload protein comprises culturing the engineered bacteria under conditions sufficient to express the recombinant polypeptide of any one of embodiments 18-30, wherein the presence of the pre-protein signal peptide induces secretion of the payload protein to a culture media, to the bacteria cell periplasm, or a combination thereof.
42. The method of embodiment 41, wherein the presence of the pre-protein signal peptide induces secretion of the payload protein to the periplasm.
43. The method of any one of embodiments 40-42, wherein the pre-protein signal peptide increases secretion of the payload protein as compared to native signal peptides.
44. The method of any one of embodiments 40-43, wherein the bacteria is of the genus Escherichia.
45. The method of any one of embodiments 40-44, wherein the bacteria is selected from the group consisting of E. alberii, E. fergusonii, E. hermannii, E. marmotae, and E. coli.
46. The method of any one of embodiments 40-45, wherein the bacteria is E. coli.
47. The method of any one of embodiments 40-46, wherein the culturing comprises incubating the engineered bacteria in culture media.
48. The method of any one of embodiments 40-47, wherein the method further comprises recovering or purifying the payload protein from the culture media, the cell periplasm, or a combination thereof.
49. The method of any one of embodiments 40-48, wherein Z₁ is selected from the group consisting of an antiviral, insulin, an incretin, an enzyme, an enzyme inhibitor, a hormone, a cytokine, an antibody, a single domain antibody fragment, a single chain variable antibody fragment, an antimicrobial peptide, a mucosal protein, pesticide, bactericide herbicide, fungicide, nematicide, miticide, plant growth regulator, plant growth stimulator or fertilizer, a vaccine, a diagnostic protein, a feed conversion enzyme, a flavoring, a nutritional protein, venom peptides, endoglucanase, restriction enzymes, human growth hormone, Beta-lactamase, luciferase (such as, but not limited to, Renilla luciferase or NanoLuc luciferase), phytase, cellulase, chitinase, phosphatase, catalase, urokinase, tissue plasminogen activator, apolipoprotein, beta-glucosidases, hemicellulases, lignocellulose oxireductases, DNAses, NADPH dehydrogenase, alcohol oxidase, pyruvate decarboxylase, formolase, alpha-ketoglutarate dehydrogenase, branched chain alpha-ketoacid decarboxylase, copper radical oxidase, galactose oxidase, glycerol oxidase, amine oxidase, glyoxalase, amino monoaxidase, ethylene glycol oxidase, alditol oxidase, or 2-oxoglutarate dehydrogenase.
50. The method of any one of embodiments 40-48, wherein Z₁ is selected from the group consisting of an antiviral, insulin, an incretin, an enzyme, an enzyme inhibitor, a hormone, a cytokine, an antibody, a single domain antibody fragment, a single chain variable antibody fragment, an antimicrobial peptide, a mucosal protein, pesticide, bactericide herbicide, fungicide, nematicide, miticide, plant growth regulator, plant growth stimulator or fertilizer, a vaccine, a diagnostic protein, a feed conversion enzyme, a flavoring, or a nutritional protein.
51. The method of any one of embodiments 40-48, wherein Z₁ is selected from the group consisting of venom peptides, endoglucanase, restriction enzymes, human growth hormone, Beta-lactamase, luciferase (such as, but not limited to, Renilla luciferase or NanoLuc luciferase), phytase, cellulase, chitinase, phosphatase, catalase, urokinase, tissue plasminogen activator, apolipoprotein, beta-glucosidases, hemicellulases, lignocellulose oxireductases, DNAses, NADPH dehydrogenase, alcohol oxidase, pyruvate decarboxylase, formolase, alpha-ketoglutarate dehydrogenase, branched chain alpha-ketoacid decarboxylase, copper radical oxidase, galactose oxidase, glycerol oxidase, amine oxidase, glyoxalase, amino monoaxidase, ethylene glycol oxidase, alditol oxidase, or 2-oxoglutarate dehydrogenase.
52. The method of any one of embodiments 40-48, wherein Z₁ comprises an amino acid sequence having at least 90%, 91%, 92%, 93%, 94%, 95%, 96%, 97%, 98%, 99%, or 100% identity to an amino acid sequence of SEQ ID NO: 28, 29, 30, 31, 32, 33, 34, 35, 36, 37, 38, 39, 40, 41, 42, 43, 44, or 72.
53. A method of producing an industrial commodity protein comprising:
   i) transfecting a bacterium with a nucleic acid molecule encoding for a recombinant polypeptide comprising a formula of X₁—Z₁ wherein:
      a) X₁ is a pre-protein signal peptide, and
      b) Z₁ is a payload protein comprising an industrial commodity protein.
   thereby producing an engineered bacterium comprising the nucleic acid molecule;
   ii) culturing the engineered bacteria comprising the nucleic acid molecule under conditions sufficient to grow the bacteria, and
   iii) inducing secretion of the payload protein by the bacteria.
54. The method of embodiment 53, wherein X₁ comprises an amino acid sequence of Formula I, wherein Formula I is represented as:

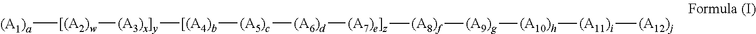

wherein:
   each w is, independently, 1 or 2;
   each x is, independently, 0 or 1;
   y is 1, 2, or 3;
   z is 2, 3, 4, 5, 6, 7, 8, 9, 10, 11, or 12;
   each b, c, d, and e is, independently, 0 or 1; and
   a, f, g, h, i, and j are each, independently, 0 or 1,
wherein:
   A₁ is methionine
   each A₂ is, independently, an amino acid selected from the group consisting of K, R, N, A, P, S, T, I, and F;
   each A₃ is, independently, an amino acid selected from the group consisting of I, L, F, V, M, Y, and H;
   each A₄ is, independently, an amino acid selected from the group consisting of L, V, C, A, F, I, T, M, P, S, G, W, Y, Q, N, R, and H;
   each A₅ is, independently, an amino acid selected from the group consisting of A, G, S, T, P, M, C, V, W, I, L, F, Y, Q, N, R, E, K, D, and H;
   each A₆ is, independently, an amino acid selected from the group consisting of G, S, N, and Q;
   each A₇ is, independently, an amino acid selected from the group consisting of S, N, T, G, V, I, Q, A, C, P, Y, M, F, and L;
   A₈ is an amino acid selected from the group consisting of A, T, G, S, P, V, I, L, Q, R, M, W, F, C, N, K, E, D, and H;
   A₉ is an amino acid selected from the group consisting of Q, F, N, S, E, T, D, R, H, K, G, A, P, Y, M, V, W, I, and L;
   A₁₀ is an amino acid selected from the group consisting of A, T, G, S, P, V, I, L, Q, R, M, W, F, C, N, K, E, D, and H;
   A₁₁ is an amino acid selected from the group consisting of A, T, G, S, P, V, I, L, Q, R, M, W, F, C, N, K, E, D, and H; and
   A₁₂ is an amino acid selected from the group consisting of D, E, Q, N, S, H, T, R, K, G, A, C, Y, P, M, V, W, I, and L.
55. The method of embodiment 54, wherein
   A₁ is methionine;
   each A₂ is, independently, an amino acid selected from the group consisting of K, R, and N;
   each A₃ is, independently, isoleucine (I);
   each A₄ is, independently, an amino acid selected from the group consisting of L, V, C, and A;
   each A₅ is, independently, an amino acid selected from the group consisting of A, G, and S;
   each A₆ is, independently, an amino acid selected from the group consisting of G, S, N, and Q;
   each A₇ is, independently, an amino acid selected from the group consisting of S, N, T, G, V, and I;
   A₈ is an amino acid selected from the group consisting of A, T, G, and S;
   A₉ is an amino acid selected from the group consisting of Q, and F;
   A₁₀ is an amino acid selected from the group consisting of A, T, G, and S;
   A₁₁ is an amino acid selected from the group consisting of A, T, G, and S; and/or
   A₁₂ is an amino acid selected from the group consisting of D, ϵ. Q, N, S, H, T, R, K, G, A, C, Y, P, M, V, W, I, and L.
56. The method of embodiment 54 or embodiment 55, wherein the pre-protein signal peptide X₁ given by Formula I comprises a minimum number of amino acids, a maximum number of amino acids, or both a minimum and maximum number of amino acids.
57. The method of any one of embodiments 54-56, wherein the pre-protein signal peptide X₁ given by Formula I comprises a minimum of 15 amino acids.
58. The method of any one of embodiments 54-56, wherein the pre-protein signal peptide X₁ given by Formula I comprises a maximum of 45 amino acids.
59. The method of any one of embodiments 54-56, wherein the pre-protein signal peptide X₁ given by Formula I comprises a minimum of 15 amino acids and a maximum of 45 amino acids.
60. The method of any one of embodiments 53-59, wherein X₁ comprises an amino acid sequence having at least 90%, 91%, 92%, 93%, 94%, 95%, 96%, 97%, 98%, 99%, or 100% identity to an amino acid sequence of SEQ ID NO: 1, 2, 3, or 4.
61. The method of any one of embodiments 53-60, wherein the pre-protein signal peptide X₁ increases the secretion of the payload protein Z₁ as compared to native signal peptides.
62. The method of any one of embodiments 53-61, wherein Z₁ is selected from the group consisting of amylases, alpha-amylases, xylanases, lichenases, lipases, pectinases, and cellulases.
63. The method of any one of embodiments 53-61, wherein Z₁ is an amylase.
64. The method of any one of embodiments 53-61, wherein Z₁ is an alpha-amylase.
65. The method of any one of embodiments 53-61, wherein Z₁ is a xylanase.
66. The method of any one of embodiments 53-61, wherein Z₁ is a lichenase.
67. The method of any one of embodiments 53-61, wherein Z₁ is a lipase.
68. The method of any one of embodiments 53-61, wherein Z₁ is a pectinase.
69. The method of any one of embodiments 53-61, wherein Z₁ is a cellulase.
70. The method of any one of embodiments 53-61, wherein Z₁ comprises an amino acid sequence having at least 90%, 91%, 92%, 93%, 94%, 95%, 96%, 97%, 98%, 99%, or 100% identity to an amino acid sequence of SEQ ID NO: 28, 29, 30, 31, 32, 33, 34, 35, 36, 37, 38, 39, 40, 41, 42, 43, 44, or 72.
71. The method of any one of embodiments 53-70, wherein inducing secretion of the payload protein comprises culturing the engineered bacteria under conditions sufficient to express the payload protein, wherein the presence of the pre-protein signal peptide induces secretion of the payload protein to a culture media, to the bacteria cell periplasm, or a combination thereof.
72. The method of embodiment 71 wherein the presence of the pre-protein signal peptide induces secretion of the payload protein to the periplasm.
73. The method of any one of embodiments 53-72, wherein the bacteria is of the genus Escherichia.
74. The method of any one of embodiments 53-73, wherein the bacteria is selected from the group consisting of E. alberii, E. fergusonii, E. hermannii, E. marmotae, and E. coli.
75. The method of any one of embodiments 53-74, wherein the bacteria is E. coli.
76. The method of any one of embodiments 53-75, wherein the culturing comprises incubating the engineered bacteria in culture media.
77. The method of any one of embodiments 53-76, wherein the method further comprises recovering or purifying the payload protein from the culture media, the cell periplasm, or a combination thereof.
78. A method for treating a disease or a condition in a subject in need thereof comprising administering to the subject a therapeutically effective amount of the engineered bacteria of any one of embodiments 31-39.
79. The method of embodiment 78, wherein the disease or condition is an infection, an autoimmune disease, enzymatic deficiency, diabetes, obesity, a metabolic disorder, intestinal bacterial overgrowth, enteric infection, bacterial vaginosis, inflammatory bowel disease, irritable bowel syndrome, small bowel syndrome, Celiac disease, gluten intolerance, colitis, peptic ulcer, or another GI condition or disorder.
80. The method of embodiment 78 or 79, wherein the administration is oral administration, local administration, or topical administration.

### EXAMPLES

Although the embodiments presented in the present application have been described in considerable detail, other versions are possible. Therefore, the spirit and scope of the appended claims should not be limited to the description and the embodiments contained within the specification. Various aspects of the present application will be illustrated with reference to the following non-limiting examples:

### Example 1—Use of Novel Signal Peptides to Increase Export of Luciferase to the Periplasm

As a proof of concept, constructs were designed for periplasmic export of Nano Luciferase (NanoLuc) from E. coli. Expression constructs were designed where the signal peptides represented by SEQ ID NO: 1, SEQ ID NO: 2, and SEQ ID NO: 3 were fused to the N-terminus of NanoLuc. As a control, constructs were also designed wherein the naturally occurring signal peptides sourced from proteins PelB, OmpA, and MalE (signal peptides of SEQ ID NO: 66, SEQ ID NO: 68, and SEQ ID NO: 70, respectively) were fused to the N-terminus of NanoLuc. The signal peptides of PelB, OmpA, and MalE are considered gold-standard signal peptides and are commonly used in the field for periplasmic export of heterologous proteins.

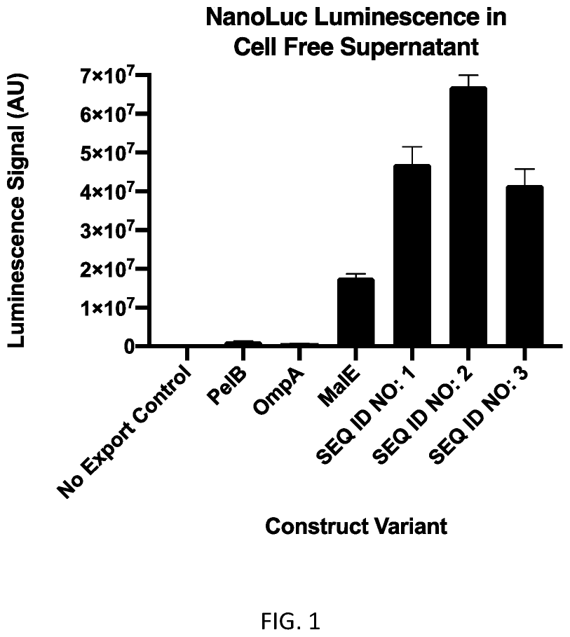

In brief, E. coli expressing each construct as well as a negative control (empty plasmid) were seeded at an optical density of 0.002 and incubated at 37° C. with shaking for 16 hours. 5 μL of culture volume was then removed from each culture and diluted 1:20 in phosphate buffered saline. The diluted fractions were centrifuged, and 10 μL of the resulting cell free culture supernatant was then assessed using the Promega Nano-Glo® Luciferase Assay System Kit. The results are shown in FIG. 1 and in Table 13 below:

TABLE 13
Fold improvement over:

| | PelB | OmpA | MalE |
|---|---|---|---|
| SEQ ID NO: 1 | 36.1X | 67.9X | 2.65X |
| SEQ ID NO: 2 | 51.5X | 96.7X | 3.78X |
| SEQ ID NO: 3 | 31.9X | 60.0X | 2.34X |

The results of the present example demonstrate the surprising result that the synthetic signal peptides of the present disclosure result in increased export of the reporter protein, NanoLuc, over three standard signal peptides used in the field. All three synthetic signal peptides of the present example drastically outperform the PelB and OmpA signal peptides and further provide at least a 2 fold increase over the third signal peptide, MalE. As NanoLuc is commonly used in the field to assess protein export, thus this supports that the synthetic signal peptides of the present disclosure will outperform standard signal peptides regardless of payload protein expressed.

### Example 2—Use of Novel Signal Peptides to Increase Export of Enoglucanases to the Periplasm

Fusion construct will be constructed testing the capability of the various signal peptides disclosed herein to promote periplasmic expression of an endoglucanase. As a control, periplasmic expression will be measured against expression constructs generating the endoglucanase without a signal peptide and generating the endoglucanase with a cellular export sequence, i.e. secretion to the culture media. Expression of the endoglucanase in the i) cell culture media, ii) periplasm, and iii) bacteria cytosol will be compared.

### Example 3—Use of Novel Signal Peptides to Increase Export of Beta-Glucosidases to the Periplasm

Fusion construct will be constructed testing the capability of the various signal peptides disclosed herein to promote periplasmic expression of a beta-glucosidase. As a control, periplasmic expression will be measured against expression constructs generating the beta-glucosidase without a signal peptide and generating the beta-glucosidase with a cellular export sequence, i.e. secretion to the culture media. Expression of the beta-glucosidase in the i) cell culture media, ii) periplasm, and iii) bacteria cytosol will be compared.

### Example 4—Use of Novel Signal Peptides to Increase Export of Cellulases to the Periplasm

Fusion construct will be constructed testing the capability of the various signal peptides disclosed herein to promote periplasmic expression of a cellulase. As a control, periplasmic expression will be measured against expression constructs generating the cellulase without a signal peptide and generating the cellulase with a cellular export sequence, i.e. secretion to the culture media. Expression of the cellulase in the i) cell culture media, ii) periplasm, and iii) bacteria cytosol will be compared.

### Example 5—Use of Novel Signal Peptides to Increase Export of Hemicellulases to the Periplasm

Fusion construct will be constructed testing the capability of the various signal peptides disclosed herein to promote periplasmic expression of a hemicellulase. As a control, periplasmic expression will be measured against expression constructs generating the hemicellulase without a signal peptide and generating the hemicellulase with a cellular export sequence, i.e. secretion to the culture media. Expression of the hemicellulase in the i) cell culture media, ii) periplasm, and iii) bacteria cytosol will be compared.

### Example 6—Use of Novel Signal Peptides to Increase Export of Lignocellulose Oxireductase to the Periplasm

Fusion construct will be constructed testing the capability of the various signal peptides disclosed herein to promote periplasmic expression of a lignocellulose oxireductase. As a control, periplasmic expression will be measured against expression constructs generating the lignocellulose oxireductase without a signal peptide and generating the lignocellulose oxireductase with a cellular export sequence, i.e. secretion to the culture media. Expression of the lignocellulose oxireductase in the i) cell culture media, ii) periplasm, and iii) bacteria cytosol will be compared.

### Example 7—Use of Novel Signal Peptides to Increase Export of DNAses to the Periplasm

Fusion construct will be constructed testing the capability of the various signal peptides disclosed herein to promote periplasmic expression of a DNAse. As a control, periplasmic expression will be measured against expression constructs generating the DNAse without a signal peptide and generating the DNAse with a cellular export sequence, i.e. secretion to the culture media. Expression of the DNAse in the i) cell culture media, ii) periplasm, and iii) bacteria cytosol will be compared.

### Example 8—Use of Novel Signal Peptides to Increase Export of Restriction Enzymes to the Periplasm

Fusion construct will be constructed testing the capability of the various signal peptides disclosed herein to promote periplasmic expression of a restriction enzyme. As a control, periplasmic expression will be measured against expression constructs generating the restriction enzyme without a signal peptide and generating the restriction enzyme with a cellular export sequence, i.e. secretion to the culture media. Expression of the restriction enzyme in the i) cell culture media, ii) periplasm, and iii) bacteria cytosol will be compared.

### Example 9—Use of Novel Signal Peptides to Produce Disulfide Bond Containing Single Domain Antibody Fragments

Fusion construct will be constructed testing the capability of the various signal peptides disclosed herein to promote periplasmic expression of single domain antibody fragments, which are known to require proper disulfide bond formation for proper function. As a control, periplasmic expression will be measured against expression constructs generating the single domain antibody fragments without a signal peptide and generating the single domain antibody fragments with a cellular export sequence, i.e. secretion to the culture media. Proper function of single domain antibody fragments will be determined via known methods.

### Example 10—Use of Novel Signal Peptides to Produce Disulfide Bond Containing Single Chain Variable Antibody Fragments

Fusion construct will be constructed testing the capability of the various signal peptides disclosed herein to promote periplasmic expression of single chain variable antibody fragments, which are known to require proper disulfide bond formation for proper function. As a control, periplasmic expression will be measured against expression constructs generating the single chain variable antibody fragments without a signal peptide and generating the single chain variable antibody fragments with a cellular export sequence, i.e. secretion to the culture media. Proper function of single chain variable antibody fragments will be determined via known methods.

### Example 11—Use of Novel Signal Peptides to Produce Disulfide Bond Containing Insulin

Fusion construct will be constructed testing the capability of the various signal peptides disclosed herein to promote periplasmic expression of insulin, which is known to require proper disulfide bond formation for proper function. As a control, periplasmic expression will be measured against expression constructs generating the insulin without a signal peptide and generating the insulin with a cellular export sequence, i.e. secretion to the culture media. Proper function of the insulin will be determined via known methods.

### Example 12—Use of Novel Signal Peptides to Produce Disulfide Bond Containing Human Growth Hormone

Fusion construct will be constructed testing the capability of the various signal peptides disclosed herein to promote periplasmic expression of human growth hormone, which are known to require proper disulfide bond formation for proper function. As a control, periplasmic expression will be measured against expression constructs generating the human growth hormone without a signal peptide and generating the human growth hormone with a cellular export sequence, i.e. secretion to the culture media. Proper function of human growth hormone will be determined via known methods.

1. (canceled)

2. A pre-protein signal peptide comprising an amino acid sequence having at least 90% identity to the amino acid sequence selected from the group consisting of SEQ ID NO: 1, 2, 3, or 4.

3. The pre-protein signal peptide of claim 2, wherein the signal peptide comprises an amino acid sequence having at least 95% identity to the amino acid sequence selected from the group consisting of SEQ ID NO: 1, 2, 3, or 4.

4. (canceled)

5. A recombinant polypeptide comprising a formula of X₁—Z₁ wherein:
X₁ is the pre-protein signal peptide of claim 2, and
Z₁ is a payload protein.

6.-7. (canceled)

8. The recombinant polypeptide of claim 5, wherein Z₁ is selected from the group consisting of an antiviral, insulin, an incretin, an enzyme, an enzyme inhibitor, a hormone, a cytokine, an antibody, a single domain antibody fragment, a single chain variable antibody fragment, an antimicrobial peptide, a mucosal protein, pesticide, bactericide herbicide, fungicide, nematicide, miticide, plant growth regulator, plant growth stimulator or fertilizer, a vaccine, a diagnostic protein, a feed conversion enzyme, a flavoring, a nutritional protein, venom peptides, endoglucanase, restriction enzymes, human growth hormone, Beta-lactamase, luciferase, phytase, cellulase, chitinase, phosphatase, catalase, urokinase, tissue plasminogen activator, apolipoprotein, beta-glucosidases, hemicellulases, lignocellulose oxireductases, DNAses, NADPH dehydrogenase, alcohol oxidase, pyruvate decarboxylase, formolase, alpha-ketoglutarate dehydrogenase, branched chain alpha-ketoacid decarboxylase, copper radical oxidase, galactose oxidase, glycerol oxidase, amine oxidase, glyoxalase, amino monoaxidase, ethylene glycol oxidase, alditol oxidase, or 2-oxoglutarate dehydrogenase.

9.-11. (canceled)

12. An engineered bacterium comprising a heterologous nucleic acid molecule encoding a polypeptide having a formula of X₁—Z₁, wherein:
X₁ is the pre-protein signal peptide of claim 2, and
Z₁ is a payload protein.

13. The engineered bacterium of claim 12, wherein the bacteria are Escherichia bacteria.

14. The engineered bacterium of claim 12, wherein the bacteria is selected from the group consisting of E. alberii, E. fergusonii, E. hermannii, E. marmotae, and E. coli.

15. (canceled)

16. The engineered bacterium of claim 12, wherein Z₁ is selected from the group consisting of an antiviral, insulin, an incretin, an enzyme, an enzyme inhibitor, a hormone, a cytokine, an antibody, a single domain antibody fragment, a single chain variable antibody fragment, an antimicrobial peptide, a mucosal protein, pesticide, bactericide herbicide, fungicide, nematicide, miticide, plant growth regulator, plant growth stimulator or fertilizer, a vaccine, a diagnostic protein, a feed conversion enzyme, a flavoring, a nutritional protein, venom peptides, endoglucanase, restriction enzymes, human growth hormone, Beta-lactamase, luciferase, phytase, cellulase, chitinase, phosphatase, catalase, urokinase, tissue plasminogen activator, apolipoprotein, beta-glucosidases, hemicellulases, lignocellulose oxireductases, DNAses, NADPH dehydrogenase, alcohol oxidase, pyruvate decarboxylase, formolase, alpha-ketoglutarate dehydrogenase, branched chain alpha-ketoacid decarboxylase, copper radical oxidase, galactose oxidase, glycerol oxidase, amine oxidase, glyoxalase, amino monoaxidase, ethylene glycol oxidase, alditol oxidase, or 2-oxoglutarate dehydrogenase.

17.-19. (canceled)

20. A method for producing a payload protein, comprising:
i) transfecting a bacterium with a nucleic acid molecule encoding for the recombinant polypeptide of claim 5 to produce an engineered bacterium comprising the nucleic acid molecule;
ii) culturing the engineered bacteria comprising the nucleic acid molecule under conditions sufficient to grow the bacteria, and
iii) inducing secretion of the payload protein by the bacteria.

21. The method of claim 20, wherein inducing secretion of the payload protein comprises culturing the engineered bacteria under conditions sufficient to express the recombinant polypeptide of claim 5, wherein the presence of the pre-protein signal peptide induces secretion of the payload protein to a culture media, to the bacteria cell periplasm, or a combination thereof.

22.-23. (canceled)

24. The method of claim 20, wherein the bacteria is of the genus Escherichia.

25. The method of claim 20, wherein the bacteria is selected from the group consisting of E. alberii, E. fergusonii, E. hermannii, E. marmotae, and E. coli.

26.-27. (canceled)

28. The method of claim 20, wherein the method further comprises recovering or purifying the payload protein from the culture media, the cell periplasm, or a combination thereof.

29. The method of claim 20, wherein Z₁ is selected from the group consisting of an antiviral, insulin, an incretin, an enzyme, an enzyme inhibitor, a hormone, a cytokine, an antibody, a single domain antibody fragment, a single chain variable antibody fragment, an antimicrobial peptide, a mucosal protein, pesticide, bactericide herbicide, fungicide, nematicide, miticide, plant growth regulator, plant growth stimulator or fertilizer, a vaccine, a diagnostic protein, a feed conversion enzyme, a flavoring, a nutritional protein, venom peptides, endoglucanase, restriction enzymes, human growth hormone, Beta-lactamase, luciferase, phytase, cellulase, chitinase, phosphatase, catalase, urokinase, tissue plasminogen activator, apolipoprotein, beta-glucosidases, hemicellulases, lignocellulose oxireductases, DNAses, NADPH dehydrogenase, alcohol oxidase, pyruvate decarboxylase, formolase, alpha-ketoglutarate dehydrogenase, branched chain alpha-ketoacid decarboxylase, copper radical oxidase, galactose oxidase, glycerol oxidase, amine oxidase, glyoxalase, amino monoaxidase, ethylene glycol oxidase, alditol oxidase, or 2-oxoglutarate dehydrogenase.

30.-31. (canceled)

32. A method of producing an industrial commodity protein comprising:
i) transfecting a bacterium with a nucleic acid molecule encoding for a recombinant polypeptide comprising a formula of X₁—Z₁ wherein:
a) X₁ is the pre-protein signal peptide of claim 2, and
b) Z₁ is a payload protein comprising an industrial commodity protein.
thereby producing an engineered bacterium comprising the nucleic acid molecule;
ii) culturing the engineered bacteria comprising the nucleic acid molecule under conditions sufficient to grow the bacteria, and
iii) inducing secretion of the payload protein by the bacteria.

33.-35. (canceled)

36. The method of claim 32, wherein Z₁ is selected from the group consisting of amylases, alpha-amylases, xylanases, lichenases, lipases, pectinases, and cellulases.

37. The method of claim 32, wherein Z₁ comprises an amino acid sequence having at least 90% identity to an amino acid sequence of SEQ ID NO: 28, 29, 30, 31, 32, 33, 34, 35, 36, 37, 38, 39, 40, 41, 42, 43, 44, or 72.

38.-39. (canceled)

40. The method of claim 32, wherein the bacteria is of the genus Escherichia.

41. The method of claim 32, wherein the bacteria is selected from the group consisting of E. alberii, E. fergusonii, E. hermannii, E. marmotae, and E. coli.

42.-44. (canceled)

45. A method for treating a disease or a condition in a subject in need thereof comprising administering to the subject a therapeutically effective amount of the engineered bacteria of claim 12.

46.-47. (canceled)


SEQUENCE LISTING (217 sequences)

SEQ ID NO: 1 - protein, 24 aa, synthetic construct
MKKLLALGLLALGLLLSSSAQAAD

SEQ ID NO: 2 - protein, 24 aa, synthetic construct
MKKLLASLVLALLLLASSSAFAAD

SEQ ID NO: 3 - protein, 25 aa, synthetic construct
MKKNLLLASLLLLLALASGSAQAAD

SEQ ID NO: 4 - protein, 25 aa, synthetic construct
MKKIKLLLLLLLLALLLSGSAFAAD

SEQ ID NO: 5 - DNA, 72 bp, synthetic construct
  source 1..72: mol_type = other DNA
atgaaaaaactgctggcgctgggcctgctggcgctgggcctgctgctgagcagcagcgcgcaggcggcggat

SEQ ID NO: 6 - DNA, 72 bp, synthetic construct
  source 1..72: mol_type = other DNA
atgaaaaaactgctggcgagcctggtgctggcgctgctgctgctggcgagcagcagcgcgtttgcggcggat

SEQ ID NO: 7 - DNA, 75 bp, synthetic construct
  source 1..75: mol_type = other DNA
atgaaaaaaaacctgctgctggcgagcctgctgctgctgctggcgctggcgagcggcagcgcgcaggcggcggat

SEQ ID NO: 8 - DNA, 75 bp, synthetic construct
  source 1..75: mol_type = other DNA
atgaaaaaaattaaactgctgctgctgctgctgctgctggcgctgctgctgagcggcagcgcgtttgcggcggat

SEQ ID NO: 9 - protein, 9 aa, synthetic construct
LEISSTCDA

SEQ ID NO: 10 - DNA, 27 bp, synthetic construct
  source 1..27: mol_type = other DNA
ttggaaatctcctcaacatgcgatgcc

SEQ ID NO: 11 - protein, 25 aa, synthetic construct
FCTFFEKHHRKWDILLEKSTGVMEA

SEQ ID NO: 12 - DNA, 75 bp, synthetic construct
  source 1..75: mol_type = other DNA
ttctgtacttttttcgagaaacatcaccggaagtgggacatacttttagagaaatccactggggtgatggaagcg

SEQ ID NO: 13 - protein, 36 aa, synthetic construct
MFAKRFKTSLLPLFAGFLLLFHLVLAGPAAASAETA

SEQ ID NO: 14 - protein, 30 aa, synthetic construct
MVSIRRSFEAYVDDMNIITVLIPAEQKEIM

SEQ ID NO: 15 - protein, 29 aa, synthetic construct
MRSKKLWISLLFALTLIFTMAFSNMSAQA

SEQ ID NO: 16 - protein, 25 aa, synthetic construct
  VARIANT 6: note = X at position 6, 12,and 18 are all the simultaneously same amino acid, with the exception of cysteine. X at positions 6 and 18 cannot be cysteine. When X at position 12 is cysteine, X at positions 6 and 18 are leucine (see SEQ ID NO: 18)
KKAAAXAAAAAXAAWAAXAAAKKKK

SEQ ID NO: 17 - protein, 25 aa, synthetic construct
KKAAAMAAAAAMAAWAAMAAAKKKK

SEQ ID NO: 18 - protein, 25 aa, synthetic construct
KKAAALAAAAACAAWAALAAAKKKK

SEQ ID NO: 19 - protein, 4 aa, synthetic construct
KIKI

SEQ ID NO: 20 - protein, 4 aa, synthetic construct
KIRL

SEQ ID NO: 21 - protein, 6 aa, synthetic construct
KIKIKI

SEQ ID NO: 22 - protein, 6 aa, synthetic construct
KIRLNF

SEQ ID NO: 23 - protein, 15 aa, synthetic construct
LALGLLALGLLLSSS

SEQ ID NO: 24 - protein, 15 aa, synthetic construct
LASLVLALLLLASSS

SEQ ID NO: 25 - protein, 18 aa, synthetic construct
NLLLASLLLLLALASGSA

SEQ ID NO: 26 - protein, 5 aa, synthetic construct
KKIKL

SEQ ID NO: 27 - protein, 15 aa, synthetic construct
LLLLLLLALLLSGSA

SEQ ID NO: 28 - protein, 585 aa, synthetic construct
APVNTTTEDETAQIPAEAVIGYSDLEGDFDVAVLPFSNSTNNGLLFINTTIASIAAKEEGVSLDKREEGEPKSMTNETSDRPLVHFTPNKGWMNDPNGLWYDEKDAKWHLYFQYNPNDTVWGTPLFWGHATSDDLTNWEDQPIAIAPKRNDSGAFSGSMVVDYNNTSGFFNDTIDPRQRCVAIWTYNTPESEEQYISYSLDGGYTFTEYQKNPVLAANSTQFRDPKVFWYEPSQKWIMTAAKSQDYKIEIYSSDDLKSWKLESAFANEGFLGYQYECPGLIEVPTEQDPSKSYWVMFISINPGAPAGGSFNQYFVGSFNGTHFEAFDNQSRVVDFGKDYYALQTFFNTDPTYGSALGIAWASNWEYSAFVPTNPWRSSMSLVRKFSLNTEYQANPETELINLKAEPILNISNAGPWSRFATNTTLTKANSYNVDLSNSTGTLEFELVYAVNTTQTISKSVFADLSLWFKGLEDPEEYLRMGFEVSASSFFLDRGNSKVKFVKENPYFTNRMSVNNQPFKSENDLSYYKVYGLLDQNILELYFNDGDVVSTNTYFMTTGNALGSVNMTTGVDNLFYIDKFQVREVK

SEQ ID NO: 29 - protein, 513 aa, synthetic construct
SMTNETSDRPLVHFTPNKGWMNDPNGLWYDEKDAKWHLYFQYNPNDTVWGTPLFWGHATSDDLTNWEDQPIAIAPKRNDSGAFSGSMVVDYNNTSGFFNDTIDPRQRCVAIWTYNTPESEEQYISYSLDGGYTFTEYQKNPVLAANSTQFRDPKVFWYEPSQKWIMTAAKSQDYKIEIYSSDDLKSWKLESAFANEGFLGYQYECPGLIEVPTEQDPSKSYWVMFISINPGAPAGGSFNQYFVGSFNGTHFEAFDNQSRVVDFGKDYYALQTFFNTDPTYGSALGIAWASNWEYSAFVPTNPWRSSMSLVRKFSLNTEYQANPETELINLKAEPILNISNAGPWSRFATNTTLTKANSYNVDLSNSTGTLEFELVYAVNTTQTISKSVFADLSLWFKGLEDPEEYLRMGFEVSASSFFLDRGNSKVKFVKENPYFTNRMSVNNQPFKSENDLSYYKVYGLLDQNILELYFNDGDVVSTNTYFMTTGNALGSVNMTTGVDNLFYIDKFQVREVK

SEQ ID NO: 30 - protein, 130 aa, synthetic construct
KVFERCELARTLKRLGMDGYRGISLANWMCLAKWESGYNTRATNYNAGDRSTDYGIFQINSRYWCNDGKTPGAVNACQLSCSALLQDNIADAVACAKRVVRDPQGIRAWVAWRNRCQNRDVRQYVQGCGV

SEQ ID NO: 31 - protein, 985 aa, synthetic construct
IKHRLNGFTILEHPDPAKRDLLQDIVTWDDKSLFINGERIMLFSGEVHPFRLPVPSLWLDIFHKIRALGFNCVSFYIDWALLEGKPGDYRAEGIFALEPFFDAAKEAGIYLIARPGSYINAEVSGGGFPGWLQRVNGTLRSSDEPFLKATDNYIANAAAAVAKAQITNGGPVILYQPENEYSGGCCGVKYPDADYMQYVMDQARKADIVVPFISNDASPSGHNAPGSGTSAVDIYGHDSYPLGFDCANPSVWPEGKLPDNFRTLHLEQSPSTPYSLLEFQAGAFDPWGGPGFEKCYALVNHEFSRVFYRNDLSFGVSTFNLYMTFGGTNWGNLGHPGGYTSYDYGSPITETRNVTREKYSDIKLLANFVKASPSYLTATPRNLTTGVYTDTSDLAVTPLIGDSPGSFFVVRHTDYSSQESTSYKLKLPTSAGNLTIPQLEGTLSLNGRDSKIHVVDYNVSGTNIIYSTAEVFTWKKFDGNKVLVLYGGPKEHHELAIASKSNVTIIEGSDSGIVSTRKGSSVIIGWDVSSTRRIVQVGDLRVFLLDRNSAYNYWVPELPTEGTSPGFSTSKTTASSIIVKAGYLLRGAHLDGADLHLTADFNATTPIEVIGAPTGAKNLFVNGEKASHTVDKNGIWSSEVKYAAPEIKLPGLKDLDWKYLDTLPEIKSSYDDSAWVSADLPKTKNTHRPLDTPTSLYSSDYGFHTGYLIYRGHFVANGKESEFFIRTQGGSAFGSSVWLNETYLGSWTGADYAMDGNSTYKLSQLESGKNYVITVVIDNLGLDENWTVGEETMKNPRGILSYKLSGQDASAITWKLTGNLGGEDYQDKVRGPLNEGGLYAERQGFHQPQPPSESWESGSPLEGLSKPGIGFYTAQFDLDLPKGWDVPLYFNFGNNTQAARAQLYVNGYQYGKFTGNVGPQTSFPVPEGILNYRGTNYVALSLWALESDGAKLGSFELSYTTPVLTGYGNVESPEQPKYEQRKGAY

SEQ ID NO: 32 - protein, 114 aa, synthetic construct
EVQLVESGGGLVQPGGSLRLSCAASGFTFSDYWMYWVRQAPGKGLEWVSEINTNGLITKYPDSVGRFTISRDNAKNTLYLQMNSLRPEDTAVYYCARSPSGFNRGQGTLVTVSS

SEQ ID NO: 33 - protein, 369 aa, synthetic construct
IEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTRITK

SEQ ID NO: 34 - protein, 411 aa, synthetic construct
AQSEPELKLESVVIVSRHGVRAPTKATQLMQDVTPDAWPTWPVKLGELTPRGGELLAYLGHYWRQRLVADGLLPKCGCPQSGQVAILADVDERTRKTGEAFAAGLAPDCAITVHTQADTSSPDPLFNPLKTGVCQLDNANVTDAILERAGGSLADFTGHYQTAFRELERVLNFPQSNLCLKREKQDESCSLTQALPSELKVSADCVSLTGAVSLASMLTEIFLLQQAQGMPEPGWGRITDSHQWNTLLSLHNAQFDLLQRTPEVARSRATPLLDLIKTALTPHPPQKQAYGVTLPTSVLFLAGHDTNLANLGGALELNWTLPGQPDNTPPGGELVFERWRRLSDNSQWIQVSLVFQTLQQMRDKTPLSLNTPPGEVKLTLAGCEERNAQGMCSLAGFTQIVNEARIPACSL

SEQ ID NO: 35 - protein, 53 aa, synthetic construct
FVNQHLCGSHLVEALYLVCGERGFFYTPKEWKGIVEQCCTSICSLYQLENYCN

SEQ ID NO: 36 - protein, 105 aa, synthetic construct
GPETLCGAELVDALQFVCGPRGFYFNKPTGYGSSIRRAPQTGIVDECCFRSCDLRRLEMYCAPLKPTKAARSIRAQRHTDMPKTQKEVHLKNTSRGSAGNKTYRM

SEQ ID NO: 37 - protein, 130 aa, synthetic construct; identical to SEQ ID NO: 30

SEQ ID NO: 38 - protein, 499 aa, synthetic construct
MKRSISIFITCLLITLLTMGGMIASPASAAGTKTPVAKNGQLSIKGTQLVNRDGKAVQLKGISSHGLQWYGEYVNKDSLKWLRDDWGITVFRAAMYTADGGYIDNPSVKNKVKEAVEAAKELGIYVIIDWHILNDGNPNQNKEKAKEFFKEMSSLYGNTPNVIYEIANEPNGDVNWKRDIKPYAEEVISVIRKNDPDNIIIVGTGTWSQDVNDAADDQLKDANVMYALHFYAGTHGQFLRDKANYALSKGAPIFVTEWGTSDASGNGGVFLDQSREWLKYLDSKTISWVNWNLSDKQESSSALKPGASKTGGWRLSDLSASGTFVRENILGTKDSTKDIPETPSKDKPTQENGISVQYRAGDGSMNSNQIRPQLQIKNNGNTTVDLKDVTARYWYKAKNKGQNFDCDYAQIGCGNVTHKFVTLHKPKQGADTYLELGFKNGTLAPGASTGNIQLRLHNDDWSNYAQSGDYSFFKSNTFKTTKKITLYDQGKLIWGTEPN

SEQ ID NO: 39 - protein, 512 aa, synthetic construct
MKQQKRLYARLLTLLFALIFLLPHSAAAAANLNGTLMQYFEWYMPNDGQHWKRLQNDSAYLAEHGITAVWIPPAYKGTSQADVGYGAYDLYDLGEFHQKGTVRTKYGTKGELQSAIKSLHSRDINVYGDVVINHKGGADATEDVTAVEVDPADRNRVISGEHRIKAWTHFHFPGRGSTYSDFKWHWYHFDGTDWDESRKLNRIYKFQGKAWDWEVSNENGNYDYLMYADIDYDHPDVAAEIKRWGTWYANELQLDGFRLDAVKHIKFSFLRDWVNHVREKTGKEMFTVAEYWQNDLGALENYLNKTNFNHSVFDVPLHYQFHAASTQGGGYDMRKLLNSTVVSKHPLKAVTFVDNHDTQPGQSLESTVQTWFKPLAYAFILTRESGYPQVFYGDMYGTKGDSQREIPALKHKIEPILKARKQYAYGAQHDYFDHHDIVGWTREGDSSVANSGLAALITDGPGGAKRMYVGRQNAGETWHDITGNRSEPVVINSEGWGEFHVNGGSVSIYVQR

SEQ ID NO: 40 - protein, 213 aa, synthetic construct
MFKFKKNFLVGLSAALMSISLFSATASAASTDYWQNWTDGGGIVNAVNGSGGNYSVNWSNTGNFVVGKGWTTGSPFRTINYNAGVWAPNGNGYLTLYGWTRSPLIEYYVVDSWGTYRPTGTYKGTVKSDGGTYDIYTTTRYNAPSIDGDRTTFTQYWSVRQSKRPTGSNATITFSNHVNAWKSHGMNLGSNWAYQVMATEGYQSSGSSNVTVW

SEQ ID NO: 41 - protein, 242 aa, synthetic construct
MPYLKRVLLLLVTGLFMSLFAVTATASAQTGGSFFDPFNGYNSGFWQKADGYSNGNMFNCTWRANNVSMTSLGEMRLALTSPAYNKFDCGENRSVQTYGYGLYEVRMKPAKNTGIVSSFFTYTGPTDGTPWDEIDIEFLGKDTTKVQFNYYTNGAGNHEKIVDLGFDAANAYHTYAFDWQPNSIKWYVDGQLKHTATNQIPTTPGKIMMNLWNGTGVDEWLGSYNGVNPLYAHYDWVRYTKK

SEQ ID NO: 42 - protein, 212 aa, synthetic construct [sequence omitted]

SEQ ID NO: 43 - protein, 341 aa, synthetic construct
MKKLISIIFIFVLGVVGSLTAAVSAEAASALNSGKVNPLADFSLKGFAALNGGTTGGEGGQTVTVTTGDQLIAALKNKNANTPLKIYVNGTITTSNTSASKIDVKDVSNVSIVGSGTKGELKGIGIKIWRANNIIIRNLKIHEVASGDKDAIGIEGPSKNIWVDHNELYHSLNVDKDYYDGLFDVKRDAEYITFSWNYVHDGWKSMLMGSSDSDNYNRTITFHHNWFENLNSRVPSFRFGEGHIYNNYFNKIIDSGINSRMGARIRIENNLFENAKDPIVSWYSSSPGYWHVSNNKFVNSRGSMPTTSTTTYNPPYSYSLDNVDNVKSIVKQNAGVGKINP

SEQ ID NO: 44 - protein, 477 aa, synthetic construct
MKNVKKRVGVVLLILAVLGVYMLAMPANTVSAAGVPFNTKYPYGPTSIADNQSEVTAMLKAEWEDWKSKRITSNGAGGYKRVQRDASTNYDTVSEGMGYGLLLAVCFNEQALFDDLYRYVKSHFNGNGLMHWHIDANNNVTSHDGGDGAATDADEDIALALIFADKLWGSSGAINYGQEARTLINNLYNHCVEHGSYVLKPGDRWGGSSVTNPSYFAPAWYKVYAQYTGDTRWNQVADKCYQIVEEVKKYNNGTGLVPDWCTASGTPASGQSYDYKYDATRYGWRTAVDYSWFGDQRAKANCDMLTKFFARDGAKGIVDGYTIQGSKISNNHNASFIGPVAAASMTGYDLNFAKELYRETVAVKDSEYYGYYGNSLRLLTLLYITGNFPNPLSDLSGQPTPPSNPTPSLPPQVVYGDVNGDGNVNSTDLTMLKRYLLKSVTNINREAADVNRDGAINSSDMTILKRYLIKSIPHLPY

SEQ ID NO: 45 - protein, 6 aa, synthetic construct
HHHHHH

SEQ ID NO: 46 - protein, 5 aa, synthetic construct
GGGGS

SEQ ID NO: 47 - protein, 15 aa, synthetic construct
GGGGSGGGGSGGGGS

SEQ ID NO: 48 - protein, 5 aa, synthetic construct
  REPEAT 1..5: note = The sequence may be present n times, wherein n is 1, 2, 3, or 4
GGGGS

SEQ ID NO: 49 - protein, 8 aa, synthetic construct
GGGGGGGG

SEQ ID NO: 50 - protein, 6 aa, synthetic construct
GGGGGG

SEQ ID NO: 51 - protein, 15 aa, synthetic construct
EAAAKEAAAKEAAAK

SEQ ID NO: 52 - protein, 4 aa, synthetic construct
  REPEAT 1..4: note = The sequence may be present n times, wherein n is 1, 2, or 3
EAAK

SEQ ID NO: 53 - protein, 46 aa, synthetic construct
AEAAAKEAAAKEAAAKEAAAKALEAEAAAKEAAAKEAAAKEAAAKA

SEQ ID NO: 54 - protein, 12 aa, synthetic construct
AEAAAKEAAAKA

SEQ ID NO: 55 - protein, 5 aa, synthetic construct
PAPAP

SEQ ID NO: 56 - protein, 4 aa, synthetic construct
  REPEAT 1..2: note = The sequence (AP) may be present n times, wherein n is 5 to 17 such that the minimum length is 10 and the maximum length is 34 amino acids
APAP

SEQ ID NO: 57 - protein, 17 aa, synthetic construct
VSQTSKLTRAETVFPDV

SEQ ID NO: 58 - protein, 6 aa, synthetic construct
PLGLWA

SEQ ID NO: 59 - protein, 6 aa, synthetic construct
RVLAEA

SEQ ID NO: 60 - protein, 10 aa, synthetic construct
EDVVCCSMSY

SEQ ID NO: 61 - protein, 8 aa, synthetic construct
GGIEGRGS

SEQ ID NO: 62 - protein, 10 aa, synthetic construct
TRHRQPRGWE

SEQ ID NO: 63 - protein, 10 aa, synthetic construct
AGNRVRRSVG

SEQ ID NO: 64 - protein, 9 aa, synthetic construct
RRRRRRRRR

SEQ ID NO: 65 - protein, 4 aa, synthetic construct
GFLG

SEQ ID NO: 66 - protein, 22 aa, synthetic construct
MKYLLPTAAAGLLLLAAQPAMA

SEQ ID NO: 67 - DNA, 66 bp, synthetic construct
  source 1..66: mol_type = other DNA
atgaaatatctgctgccgactgcggcagcgggtttactgctgctggcagcgcaaccggccatggcc

SEQ ID NO: 68 - protein, 21 aa, synthetic construct
MKKTAIAIAVALAGFATVAQA

SEQ ID NO: 69 - DNA, 63 bp, synthetic construct
  source 1..63: mol_type = other DNA
atgaaaaaaaccgcgatcgcgatcgcggttgcgctggcgggtttcgcgaccgttgcgcaggcg

SEQ ID NO: 70 - protein, 26 aa, synthetic construct
MKIKTGARILALSALTTMMFSASALA

SEQ ID NO: 71 - DNA, 78 bp, synthetic construct
  source 1..78: mol_type = other DNA
atgaaaataaaaacaggtgcacgcatcctcgcattatccgcattaacgacgatgatgttttccgcctcggctctcgcc

SEQ ID NO: 72 - protein, 170 aa, synthetic construct
VFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILA

SEQ ID NO: 73 - protein, 24 aa, synthetic construct
MFKLLALGLLALGLLLSSSAQAAD

SEQ ID NO: 74 - protein, 24 aa, synthetic construct
MKKLLALGLLALGLLLSSSAQAAD

SEQ ID NO: 75 - protein, 24 aa, synthetic construct
MKKLLALGLLALGLLLSSSVQAAD

SEQ ID NO: 76 - protein, 24 aa, synthetic construct
MKKLLALGLLAVGLLLSSSAQAAD

SEQ ID NO: 77 - protein, 24 aa, synthetic construct
MKKLLSLGLLALGLLLSSSAQAAE

SEQ ID NO: 78 - protein, 24 aa, synthetic construct
MKKLLALGLIALGLLLSSSAQAAD

SEQ ID NO: 79 - protein, 24 aa, synthetic construct
MKKLLLLGLLALGLLLSSSAQAAE

SEQ ID NO: 80 - protein, 24 aa, synthetic construct
MKKLLPLGLLALGLLLSSSAQAAD

SEQ ID NO: 81 - protein, 24 aa, synthetic construct
MKKLLALGLLALGLLFSSSAQAAD

SEQ ID NO: 82 - protein, 24 aa, synthetic construct
MKKLLALGLCALGLLLSSSAHAAD

SEQ ID NO: 83 - protein, 24 aa, synthetic construct
MKKLLGLGMLALGLLLSSSAQAAE

SEQ ID NO: 84 - protein, 24 aa, synthetic construct
MFRLLLALGLLALGLLLSSAQAAD

SEQ ID NO: 85 - protein, 24 aa, synthetic construct
MKKIALLTLTLALLLSASSAFAAD

SEQ ID NO: 86 - protein, 24 aa, synthetic construct
MFKILASLVLAALLLASASAFAAD

SEQ ID NO: 87 - protein, 25 aa, synthetic construct
MKKNLLLASLLLLLALASGSAQAAD

SEQ ID NO: 88 - protein, 25 aa, synthetic construct
MFKNLLLASLLLLLALASGSAFAAT

SEQ ID NO: 89 - protein, 25 aa, synthetic construct
MFKNLLLASLLLLLALASGSAQAAD

SEQ ID NO: 90 - protein, 25 aa, synthetic construct
MKKNLLLASLLLLLALASYSALGED

SEQ ID NO: 91 - protein, 25 aa, synthetic construct
MKKNSLLASLLLLLALASGSAHAAD

SEQ ID NO: 92 - protein, 25 aa, synthetic construct
MKKNLLLFSLLLLLALASGSAQAAD

SEQ ID NO: 93 - protein, 25 aa, synthetic construct
MKRNLLLASLLLLLALASGSAQAAE

SEQ ID NO: 94 - protein, 25 aa, synthetic construct
MKKNLLLASLMGLLALASGSAQAAD

SEQ ID NO: 95 - protein, 25 aa, synthetic construct
MKKNLLLASLLLLLTLASGSAQAAD

SEQ ID NO: 96 - protein, 25 aa, synthetic construct
MKKNLLLASLMLLLALTSGSAQAAE

SEQ ID NO: 97 - protein, 25 aa, synthetic construct
MKKNLLRASLLLLLALASGSAQAAD

SEQ ID NO: 98 - protein, 25 aa, synthetic construct
MKRNLLLASLLLLLALASGSAQAAD

SEQ ID NO: 99 - protein, 25 aa, synthetic construct
MKKNLLLLSLLLLLALASGSAQAAE

SEQ ID NO: 100 - protein, 25 aa, synthetic construct
MKKNLLLASLLLLLALASGSAAAAD

SEQ ID NO: 101 - protein, 25 aa, synthetic construct
MKKNLLLASLLLLLALAPGSAQAAD

SEQ ID NO: 102 - protein, 25 aa, synthetic construct
MKKNLLLASLLLLLALASHSAQAAD

SEQ ID NO: 103 - protein, 25 aa, synthetic construct
MTKNLLLASLLLLLALASGSAQAAD

SEQ ID NO: 104 - protein, 25 aa, synthetic construct
MKKNLLLASLVLLLALASGSAHAAD

SEQ ID NO: 105 - protein, 25 aa, synthetic construct
MKANLLLASLLLLLALASGSAQAAD

SEQ ID NO: 106 - protein, 25 aa, synthetic construct
MKKNLLLASLALLLALASGSAQAAD

SEQ ID NO: 107 - protein, 25 aa, synthetic construct
MKKNLLLASLLLAMALASGSAQAAD

SEQ ID NO: 108 - protein, 25 aa, synthetic construct
MKKNLLLASLVLLLALASGSAQAAD

SEQ ID NO: 109 - protein, 25 aa, synthetic construct
MKKNLLLASLLLLLALASPSAQAAE

SEQ ID NO: 110 - protein, 25 aa, synthetic construct
MKKNLLLASLMLLLALASGMAHAAE

SEQ ID NO: 111 - protein, 25 aa, synthetic construct
MKKNILLASLLLLLALASGSAQAAD

SEQ ID NO: 112 - protein, 25 aa, synthetic construct
MKKNLLLASLLLLLALASMSAQAAD

SEQ ID NO: 113 - protein, 25 aa, synthetic construct
MKKNLLLASLLLLLAVASGSAQAAE

SEQ ID NO: 114 - protein, 25 aa, synthetic construct
MKKNLLLLSLLLLLALASGSAQAAD

SEQ ID NO: 115 - protein, 25 aa, synthetic construct
MKKFLLLASLLLLLALASGSAQAAE

SEQ ID NO: 116 - protein, 25 aa, synthetic construct
MKKNLLLAVLLLLLALASGSAQAAD

SEQ ID NO: 117 - protein, 25 aa, synthetic construct
MKKNLLFASLLLLLALASGSAQAAD

SEQ ID NO: 118 - protein, 25 aa, synthetic construct
MKKNLLLASLLLCLALASGSAQAAD

SEQ ID NO: 119 - protein, 25 aa, synthetic construct
MKKNLLLASLLLLLAFASGSAQAAD

SEQ ID NO: 120 - protein, 25 aa, synthetic construct
MKKNLLAASLLLLLALASGSAQAAD

SEQ ID NO: 121 - protein, 25 aa, synthetic construct
MKKNLALASLLLLLALASGSAQAAE

SEQ ID NO: 122 - protein, 25 aa, synthetic construct
MKKNLLLASILLLLALASGSAHAAD

SEQ ID NO: 123 - protein, 25 aa, synthetic construct
MKKNLLLASLLPLLALASGSAQAAD

SEQ ID NO: 124 - protein, 25 aa, synthetic construct
MKKNILLASLLLLLAMASGSAQAAD

SEQ ID NO: 125 - protein, 25 aa, synthetic construct
MKKNSLLASLLLLLALASGSAQAAE

SEQ ID NO: 126 - protein, 25 aa, synthetic construct
MKKNLLLASLLLVLALASGSAQAAD

SEQ ID NO: 127 - protein, 25 aa, synthetic construct
MKKNLLLASLLMALALASGSAQAAD

SEQ ID NO: 128 - protein, 25 aa, synthetic construct
MKKNLLLASLLLLLALASGSAQADD

SEQ ID NO: 129 - protein, 25 aa, synthetic construct
MKKNLLLASLLILLALASGSAQAAD

SEQ ID NO: 130 - protein, 25 aa, synthetic construct
MKKNLLLASLLLLCALASGSAQAAD

SEQ ID NO: 131 - protein, 25 aa, synthetic construct
MKKNLLLASLVLLLALASGSAQAAE

SEQ ID NO: 132 - protein, 25 aa, synthetic construct
MKKNLLLASLLLALALASGSAQAAD

SEQ ID NO: 133 - protein, 25 aa, synthetic construct
MKKNLLLASLLLLLALASGSGQAAD

SEQ ID NO: 134 - protein, 25 aa, synthetic construct
MKKNLLLASLLLLLALAAGSAQAAK

SEQ ID NO: 135 - protein, 25 aa, synthetic construct
MFKTLLLASLLLLLALASGSAQAAD

SEQ ID NO: 136 - protein, 25 aa, synthetic construct
MFKNLLLASLLLLLALASGSAQADD

SEQ ID NO: 137 - protein, 25 aa, synthetic construct
MFRNLLLLSLLLLLALASGSAQAAD

SEQ ID NO: 138 - protein, 25 aa, synthetic construct
MKKNLLLAVLLLLLALASGSVFAED

SEQ ID NO: 139 - protein, 25 aa, synthetic construct
MKKIKLLLLLLLLALLLSGSAFAAD

SEQ ID NO: 140 - protein, 25 aa, synthetic construct
MKKIKLLLLLLLLALLLSGSAFAAG

SEQ ID NO: 141 - protein, 25 aa, synthetic construct
MFKIKLLLLLLLLALLLSGSAFAAD

SEQ ID NO: 142 - protein, 25 aa, synthetic construct
MFKIKLLLLLLLLALLLSGSAFAAE

SEQ ID NO: 143 - protein, 25 aa, synthetic construct
MKKIKLLLLLLLLALLLSGSAFAAT

SEQ ID NO: 144 - protein, 25 aa, synthetic construct
MFKIRPLCLLLLLALLLSGSAFAAD

SEQ ID NO: 145 - protein, 25 aa, synthetic construct
MFRLKLLLLLLMLALLLSGSAFAAD

SEQ ID NO: 146 - protein, 25 aa, synthetic construct
MKKIKLLLLLLLLALLLSGSAQAKD

SEQ ID NO: 147 - protein, 25 aa, synthetic construct
MKKIKLLLLLLLAALLLSGSAFAAD

SEQ ID NO: 148 - protein, 25 aa, synthetic construct
MKKIKLLLLLLLLAGLLSGSAFAAD

SEQ ID NO: 149 - protein, 25 aa, synthetic construct
MKKIKLLLLLLLLALLLSLSAYAAD

SEQ ID NO: 150 - protein, 25 aa, synthetic construct
MKKIKLLLLLLLLALLLSGSVFAAD

SEQ ID NO: 151 - protein, 25 aa, synthetic construct
MKKIKLLLLLLLLALLLSGVAFAAD

SEQ ID NO: 152 - protein, 25 aa, synthetic construct
MTKIKLLLLLLLLALLLSGSAFAAD

SEQ ID NO: 153 - protein, 25 aa, synthetic construct
MKKIKLLLLLLLLALLLSLSAFAAD

SEQ ID NO: 154 - protein, 25 aa, synthetic construct
MKKIKLLLLLLLLALQLSGSAFAAD

SEQ ID NO: 155 - protein, 25 aa, synthetic construct
MKKIRLLLLLLLLALLLSGSAFAAD

SEQ ID NO: 156 - protein, 25 aa, synthetic construct
MKRIKLLMLLLLLALLLSGSAFAAD

SEQ ID NO: 157 - protein, 25 aa, synthetic construct
MKKIKLLFLLLLLALLLSGSAFAAD

SEQ ID NO: 158 - protein, 25 aa, synthetic construct
MKKIKLLLLLALLALLLSGSAFAAD

SEQ ID NO: 159 - protein, 25 aa, synthetic construct
MKKIYLLLLLLLLALLLSGSAFAAD

SEQ ID NO: 160 - protein, 25 aa, synthetic construct
MKKIKLLLMLLLLALLLAGSAFAAD

SEQ ID NO: 161 - protein, 25 aa, synthetic construct
MKKIKLLLLALLLALLLSGSAFAAD

SEQ ID NO: 162 - protein, 25 aa, synthetic construct
MKKIKLLLLLLLLALPLSGSAFAAD

SEQ ID NO: 163 - protein, 25 aa, synthetic construct
MKKLKLLLLLLLLALLLSGSAFAAD

SEQ ID NO: 164 - protein, 25 aa, synthetic construct
MKKIKLLGLLLLLAMLLSGSAFAAE

SEQ ID NO: 165 - protein, 25 aa, synthetic construct
MTKIKLLLLLLLLALLLSGSAFAAE

SEQ ID NO: 166 - protein, 25 aa, synthetic construct
MYKIKLLLLLLLLAMLLSGSAFAAE

SEQ ID NO: 167 - protein, 25 aa, synthetic construct
MKKLKLLLLLLLLALLLSGSAFAAE

SEQ ID NO: 168 - protein, 25 aa, synthetic construct
MKKIKLLLLLLLLALCLSGSAFAAD

SEQ ID NO: 169 - protein, 25 aa, synthetic construct
MKKIKLLLLLLLLSLLLSGSAFAAD

SEQ ID NO: 170 - protein, 25 aa, synthetic construct
MKKVKLLLLLLLLALLWSGSAFAAD

SEQ ID NO: 171 - protein, 25 aa, synthetic construct
MKRIKLLLLLLLLALLLSGSAFAAD

SEQ ID NO: 172 - protein, 25 aa, synthetic construct
MKKIKLLLLLLLVALLLSGSAFAAE

SEQ ID NO: 173 - protein, 25 aa, synthetic construct
MKKHKLLLLLLLLALLLSGSAFAAD

SEQ ID NO: 174 - protein, 25 aa, synthetic construct
MKKIKLLLLLLLLALLLAGSAFAAD

SEQ ID NO: 175 - protein, 25 aa, synthetic construct
MKKIKLLLLLLLLLLLLSGSAFAAD

SEQ ID NO: 176 - protein, 25 aa, synthetic construct
MKKIKLLGLLLLLALLLSGSAFAAE

SEQ ID NO: 177 - protein, 25 aa, synthetic construct
MKKIKLLLLLLLLALLVSGSAFAAD

SEQ ID NO: 178 - protein, 25 aa, synthetic construct
MKKIKLLLLLLLLALLASGSAFAAD

SEQ ID NO: 179 - protein, 25 aa, synthetic construct
MKKIKLLLLLLLLALLLSGSAFADD

SEQ ID NO: 180 - protein, 25 aa, synthetic construct
MKKIKLLLSLLLLALLLSGSAFAAD

SEQ ID NO: 181 - protein, 25 aa, synthetic construct
MNKIKLLLLLLLLALLLSGSAFAAD

SEQ ID NO: 182 - protein, 25 aa, synthetic construct
MKKIKLLLLLLLYALLLSGSAFAAD

SEQ ID NO: 183 - protein, 25 aa, synthetic construct
MKKIKLLLLLLLLALLLSGSTFAAD

SEQ ID NO: 184 - protein, 25 aa, synthetic construct
MKKIKLLLLLLLLALLLPGSAFAAD

SEQ ID NO: 185 - protein, 25 aa, synthetic construct
MKKIKLLLALLLLALLLSGSAFAAD

SEQ ID NO: 186 - protein, 25 aa, synthetic construct
MKKFKLLLLLLLLALLLSGSAFAAD

SEQ ID NO: 187 - protein, 25 aa, synthetic construct
MKKIKLLLLLLLLALLLSGSLFAAD

SEQ ID NO: 188 - protein, 25 aa, synthetic construct
MKKIKLLLLLLLLALLLSGSAFAQD

SEQ ID NO: 189 - protein, 25 aa, synthetic construct
MYKIKLLLLLLLLALLLSGSAFAAD

SEQ ID NO: 190 - protein, 25 aa, synthetic construct
MKKIKLLLLLLILALLLSGSAFAAD

SEQ ID NO: 191 - protein, 25 aa, synthetic construct
MKKVKLLLLALLLALLLSGSAFAAD

SEQ ID NO: 192 - protein, 25 aa, synthetic construct
MKKIKLILLLLLLALLLSGSAFAAD

SEQ ID NO: 193 - protein, 25 aa, synthetic construct
MKKIKLLLLALMLALLLSGSAFAAE

SEQ ID NO: 194 - protein, 25 aa, synthetic construct
MKKIKLLLLLLVLALLLSGSAFAAD

SEQ ID NO: 195 - protein, 25 aa, synthetic construct
MKKIKLLCLLLLLALLLSGSAFAAD

SEQ ID NO: 196 - protein, 25 aa, synthetic construct
MKKIKLALLLLLLALLLSGSAFAAD

SEQ ID NO: 197 - protein, 25 aa, synthetic construct
MKKIKLLLLLLLAALLLSGSAFAAG

SEQ ID NO: 198 - protein, 25 aa, synthetic construct
MKKIKLLLLALLLALLLSGSAFAAI

SEQ ID NO: 199 - protein, 25 aa, synthetic construct
MKKIKLLLLLLLLALLYSGSAFAAK

SEQ ID NO: 200 - protein, 25 aa, synthetic construct
MFKIKLLLLLLLLALLLSGSAFGAD

SEQ ID NO: 201 - protein, 25 aa, synthetic construct
MFKIKLLLLLLLLFLLLSGSAFAAD

SEQ ID NO: 202 - protein, 25 aa, synthetic construct
MFKIKLLLLLLLLSLLLSGSAFAAD

SEQ ID NO: 203 - protein, 25 aa, synthetic construct
MFKLKLLLLLLLLALLLSGSAFAAD

SEQ ID NO: 204 - protein, 25 aa, synthetic construct
MFKIKLLLLLLLLALLLPGSAFAAD

SEQ ID NO: 205 - protein, 25 aa, synthetic construct
MFKIKMLLLLLLLGLLLSGSAFAAD

SEQ ID NO: 206 - protein, 25 aa, synthetic construct
MFKIKLLLLLLLLALLLCGSAFAAD

SEQ ID NO: 207 - protein, 25 aa, synthetic construct
MFKIKLLLLLLLLALLLAGSAFAAE

SEQ ID NO: 208 - protein, 25 aa, synthetic construct
MFKIKLLLLALLLALLLSGSAFAAD

SEQ ID NO: 209 - protein, 25 aa, synthetic construct
MFKIKLLLLLLLLAALLSGSAFAAD

SEQ ID NO: 210 - protein, 25 aa, synthetic construct
MFKIKLLLLLLLLALLLSLSAFAAD

SEQ ID NO: 211 - protein, 25 aa, synthetic construct
MFKYKLLLLLLLLALLLSGSAFAAE

SEQ ID NO: 212 - protein, 25 aa, synthetic construct
MFKIKLLLLLLLLALLLPGSAFAAE

SEQ ID NO: 213 - protein, 25 aa, synthetic construct
MFKIKLLLLLLALALLLSGSAFAAD

SEQ ID NO: 214 - protein, 25 aa, synthetic construct
MFKIKLLLLALLLALLLSGSAFAAM

SEQ ID NO: 215 - protein, 25 aa, synthetic construct
MYLIKLLLLLLLLALLLAGSAFAAD

SEQ ID NO: 216 - protein, 25 aa, synthetic construct
MFKIKLLLLCLLLALLLSGSAFAEE

SEQ ID NO: 217 - protein, 25 aa, synthetic construct
MFRIKLLLLLLLLSLLLSGSAFAGE